(19) United States
(12) Patent Application Publication
(10) Pub. No.: US 2020/0283854 A1
(43) Pub. Date: Sep. 10, 2020

(54) METHOD FOR METHYLATION ANALYSIS

(71) Applicant: Clinical Genomics Pty Ltd, Elsternwick (AU)

(72) Inventors: Susanne Kartin Pedersen, New South Wales (AU); Nicola Rosalind Boulter, New South Wales (AU)

(21) Appl. No.: 16/645,749

(22) PCT Filed: Sep. 14, 2018
(86) PCT No.: PCT/AU2018/051003

§ 371 Date: Mar. 9, 2020

(30) Foreign Application Priority Data
Sep. 15, 2017 (AU) 2017903772

(51) Int. Cl. C12Q 1/6886; C12Q 1/686

(52) U.S. Cl. CPC C12Q 1/6886; C12Q 1/686; C12Q 2600/118; C12Q 2600/112; C12Q 2600/154

(57) ABSTRACT

A method and kit for assessing DNA methylation. More particularly, a method of either qualitatively or quantitatively assessing, with improved sensitivity, the cytosine methylation of either fully or partially methylated DNA. The method and kit are useful in a range of applications including, but not limited to, the diagnosis of conditions or monitoring the development of phenotypes which are characterized by cytosine methylation changes.

### FIELD OF THE INVENTION

The present invention relates generally to a method for assessing DNA methylation. More particularly, the present invention relates to a method of either qualitatively or quantitatively assessing, with improved sensitivity, the cytosine methylation of either fully or partially methylated DNA. The method of the present invention is useful in a range of applications including, but not limited to, the diagnosis of conditions or monitoring the development of phenotypes which are characterized by cytosine methylation changes.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Bibliographic details of the publications referred to by author in this specification are collected alphabetically at the end of the description.

DNA methylation is one of the most intensely studied epigenetic modifications in mammals and refers to the addition of a methyl (CH3) group to a cytosine (C) or adenine (A) nucleotides. This methyl group may be added to the fifth carbon atom of the cytosine base or the sixth nitrogen atom of the adenine base.

DNA methylation plays a role in gene regulation in animal cells. Not only is there a correlation between active gene transcription and hypo-methylation, but also transfection experiments show that the presence of methyl moieties inhibits gene expression in vivo. Furthermore, gene activation can be induced by treatment of cells with 5-azacytidine, a potent demethylating agent. Methylation appears to influence gene expression by affecting the interactions of DNA with both chromatin proteins and specific transcription factors. Although methylation patterns are very stable in somatic cells, the early embryo is characterised by large alterations in DNA methylation.

DNA methylation is therefore vital to healthy growth and development and is linked to various processes such as genomic imprinting, carcinogenesis and the suppression of repetitive elements. It also enables the expression of retroviral genes to be suppressed, along with other potentially dangerous sequences of DNA that have entered and may damage the host. In addition, DNA methylation plays an important role in the development of cancer and is a key regulator of gene transcription. Studies have shown that genes with a promoter region that contains a high concentration of 5-methylcytosine are transcriptionally silent.

Between 60% and 90% of all CpGs are methylated in mammals. Methylated cytosine residues spontaneously deaminate to form T residues over time; hence methylated CpG dinucleotides steadily deaminate to TpG dinucleotides, which is evidenced by the under-representation of CpG dinucleotides in the human genome (they occur at only 21% of the expected frequency). CpG islands are regions with a high frequency of CpG sites which are typically present at the start of many genes.

With growing evidence of the diagnostic utility of monitoring DNA methylation levels, means for reliably and accurately assessing DNA methylation is becoming increasingly important. Currently, methylation-specific PCR is a commonly used method for detecting methylated CpG sites in bisulphite-converted DNA. In this method, PCR oligonucleotide primers interrogate methylated cytosine residues in cytosine-phosphodiester-guanidine [CpG] sites. MethyLight PCR is a real-time PCR variation which, in addition to methylation specific primers, also uses a 5′-3′ hydrolysis probe for interrogation of methylated CpG sites, thereby enabling quantification.

Tissue biopsies have long served as a source of biological material for pathology testing to aid in diagnosis, prognosis, detection of residual disease and therapy selection. There is growing evidence that most types of cancers shed DNA into blood (circulating tumor DNA—ctDNA), and detection of ctDNA could have a substantial impact on morbidity and mortality. ctDNA is generally present at very low levels and is heavily fragmented, due in part to apoptosis and necrosis being the predominant source of ctDNA release (S. Jahr, 2001 et al.; K. C. A. Chan et al., 2004; F. Mouliere et al., 2011; F. Diehl et al., 2005; P. O. Delgado et al., 2013; I. B. Roninson et al., 2001). ctDNA is commonly detected by targeting tumor-specific somatic genomic alterations, such as in the KRAS, BRAF and EGFR genes, which are absent from DNA taken from matched normal cells and in the circulating cell-free DNA (ccfDNA—primary source is white blood cells) of healthy subjects. Large-scale sequencing projects such as The Cancer Genome Atlas (TCGA) and the International Cancer Genome Consortium (ICGC) have revealed that very few somatic mutations are observed in more than 5-10% of tumours of a particular tissue type (B. Vogelstein et al., 2013) and mutation patterns are highly variable in genes due to tumour heterogeneity (M. S. Lawrence et al., 2013). The diversity in mutations creates a challenge for the development of cancer diagnostic tests based on DNA sequence changes, because large proportions of the genome need to be interrogated to provide a test of adequate sensitivity. Aberrant DNA methylation is a characteristic of most types of solid cancer with common hypermethylation events occurring more frequently than most mutations. Unlike mutation targets, DNA methylation patterns are relatively stable throughout the cancer evolution from early to late stage. Therefore, methylation-based testing has clear theoretical benefits for monitoring ctDNA dynamics without the need for development of highly individualized assays (B. Vogelstein et al., 2013; S. Garrigou et al., 2016; P. Polak et al., 2015; K. Warton et al., 2015). However, ctDNA represent only one subtype of ccfDNA. Non-tumour ccfDNA is also a potential target for methylation analysis for diagnostic, prognostic or monitoring purposes. Like ctDNA, ccfDNA similarly suffers the drawbacks associated with very low copy number and therefore potentially false negative results.

However, there are certain complexities with respect to detecting low copy numbers of methylated DNA, such as ccfDNA (and in particular disease specific ccfDNA such as ctDNA). Aside from the inherent difficulty associated with the detection and amplification of very low copy number DNA molecules, it is common practice for the detection of methylation changes to pre-treat the DNA with a bisulphite solution that deaminates unmethylated cytosine to produce uracil in DNA, which is then converted to thymine during subsequent PCR amplification. Bisulphite treatment does not affect methylated cytosines and can therefore be readily detected. Consequently, because not all cytosines will be methylated, the two complementary strands in the native double-stranded molecule become two non-complementary single strands following bisulphite conversion. Accordingly, unlike with the PCR amplification of non-bisulphite treated double stranded DNA, where the forward and reverse primers will anneal to the complementary sequences of the two respective strands of double stranded DNA (dsDNA), the number of starting copies of bisulphite converted DNA are effectively immediately halved due to the loss of the complementarity of the two strands in a targeted region. Still further, there is also an inherent loss of starting material due to loss of material during the purification of the bisulphite treated DNA and/or due to fact that bisulphite treatment result in highly degraded DNA due to the high pH, high bisulphite concentration and elevated temperature required to drive the bisulphite conversion process. The degradation occurs as depurinations result in random strand breaks. Therefore, the longer the desired amplicon, the more limited the number of intact template molecules will likely be. This could lead to the failure of the PCR amplification, or the loss of quantitatively accurate information on methylation levels resulting from the limited sampling of template molecules. Finally, bisulphite treatment also causes random nicking (i.e. destruction of the phosphate backbone) of single stranded DNA molecules. If such ‘nicking’ occurs in the targeted amplicon region, this leads to non-amplifiable DNA and target detectability is further compromised. Furthermore, depending on the area of interrogation for the gene(s) of interest, the DNA will be either highly AT rich due to the bisulphite conversion process, or highly CG rich if the region occurs in a CpG island, a process that reduces genome complexity, but which leads to difficulties in designing suitable PCR assays. Where this degradation or nicking occurs in the targeted amplicon region, there occurs further loss of starting template. If the focus of analysis is ctDNA, which is already present at very low levels, this combination of factors may prove fatal to obtaining an accurate result due to the inability to reliably amplify a fragment of DNA of sufficient length to enable target specific detection.

Accordingly, there is a need to develop improved methods that enable accurate and sensitive detection of DNA methylation, thereby improving the sensitivity of the applications for DNA methylation analysis, such as diagnosis, prognosis or monitoring of disease. In work leading up to the present invention, it has been determined that the sensitivity of DNA methylation analysis, in particular quantitative analysis, is significantly improved if the amplification reaction is designed to utilise distinct sets of primers and/or probes which are themselves designed to enable the simultaneous amplification of both the target strand and the non-complementary opposite strand of the bisulphite converted DNA region of interest. This finding is entirely unexpected when one considers that targeting both strands of complementary DNA in the context of the DNA methylation analysis of many biological samples shows no increase in the copy numbers detected. The present inventors have determined, however, that where one is specifically amplifying a target input population which is very low (such as at or below the limit of detection [LOD]), not only is there an improvement in sensitivity, but the copy number which is obtained represents a doubling of the copy number which would theoretically be obtained if 100% of that same input target population was amplified from the target strand alone. However, it is known that the bisulphite conversion step which is inherent in a methylation analysis causes degradation and/or random nicking of the input DNA population, thereby severely reducing the starting amount of amplifiable DNA. Accordingly, although one might logically expect that designing an amplification reaction to amplify both the target strand and the opposite strand might double the output relative to targeting only one strand, this “doubling” would be calculated relative to the amount of amplifiable DNA present after the bisulphite conversion step, which has now been determined to degrade and/or nick significantly more DNA than was previously thought to occur. However, in the context of the present invention, it has been very unexpectedly found that the “doubling” of copy number which is observed to occur is actually a doubling of the level of pre-bisulphite treated DNA, that is the level which exists prior to the significant nicking and degradation which is known to be induced by bisulphite conversion. This result is entirely counterintuitive but highly significant since, for the first time, the presence of methylated rare copy number DNA in a sample is now routinely and reliable detectable, where it was previously the case that prior art methods were not sufficiently sensitive to detect such DNA. Still further, it should be understood that due to the actions of bisulphite degradation and nicking, the skilled person would not have considered attempting to amplify both strands as a means to improve output since the bisulphite degradation would have significantly reduced the already rare copy number DNA level, thereby rendering methylation analysis by amplification based means futile. That the present inventors have now determined that the method of the present invention in fact produces a doubling output which is at least 50% more than what was theoretically thought possible has enabled the development of an assay which can sensitively and reliably detect the methylation of low copy number DNA. This latter finding is still further unexpected when one considers that the false positive rate is not increased but the false negative rate is significantly decreased. When one considers these findings in light of the fact that it has now also been unexpectedly determined that the reduction in low copy number starting template levels due to nicking is not approximately 50%, as has previously been accepted but is, in fact, up to 90%, the improvement to the efficacy of the method is even more surprising. Prior to the development of the present invention, obtaining clinically useful results from low copy number DNA was unachievable due to the fact that there was either no amplification product produced or, even less desirably, the result obtained reflected a level of false negative results that was much higher than previously thought, thereby potentially leading to highly adverse clinical outcomes for patients relying on these results. Still further, the recognition of the occurrence of nicking (although not the extent of the problem) had previously resulted in significant efforts being undertaken to mitigate this problem. However, all such prior art efforts had focussed on optimising the bisulphite conversion step of the method. These efforts had been unsuccessful, however, since the techniques which increased bisulphite conversion efficiency inherently increased DNA degradation. Significantly, there was no recognition at that time that the amplification step itself could be optimised to achieve significantly better efficiency than the prior art bisulphite conversion optimisation efforts. Accordingly, the method of the present invention has now enabled the development of a reliable and accurate method of performing amplification based methylation analyses of low copy number DNA molecules.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

The subject specification contains nucleotide sequence information prepared using the programme PatentIn Version 3.5, presented herein after the bibliography. Each nucleotide sequence is identified in the sequence listing by the numeric indicator <210> followed by the sequence identifier (e.g. <210>1, <210>2, etc). The length, type of sequence (DNA, etc) and source organism for each sequence is indicated by information provided in the numeric indicator fields <211>, <212> and <213>, respectively. Nucleotide sequences referred to in the specification are identified by the indicator SEQ ID NO: followed by the sequence identifier (e.g. SEQ ID NO:1, SEQ ID NO:2, etc.). The sequence identifier referred to in the specification correlates to the information provided in numeric indicator field <400> in the sequence listing, which is followed by the sequence identifier (e.g. <400>1, <400>2, etc). That is SEQ ID NO:1 as detailed in the specification correlates to the sequence indicated as <400>1 in the sequence listing.

One aspect of the present invention is directed to a method of screening for the methylation of a DNA region of interest, said method comprising:
(i) contacting a DNA sample with an agent which modifies unmethylated cytosine residues wherein said sample comprises both the target strand and the opposite strand of said DNA region of interest;
(ii) contacting the DNA sample of step (i) with:
   a) a first set of forward and reverse primers designed to amplify one or more fully or partially methylated forms of the modified target strand of the DNA region of interest;
   b) a second set of forward and reverse primers designed to amplify one or more fully or partially methylated forms of the modified opposite strand of the DNA region of interest; and
   c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said gene effects the detection of said hybridised probe; and
(iv) qualitatively or quantitatively analysing the detection output of step (iii) to obtain a result which exhibits a reduced incidence of false negative results.

More particularly there is provided an improved method of screening for the methylation of a DNA region of interest, said method comprising:
(i) contacting a DNA sample with an agent which modifies unmethylated cytosine residues wherein said sample comprises both the target strand and the opposite strand of said DNA region of interest, which DNA sample comprises a low copy number of said DNA region of interest;
(ii) contacting the DNA sample of step (i) with:
   a) a first set of forward and reverse primers designed to amplify one or more fully or partially methylated forms of the modified target strand of the DNA region of interest;
   b) a second set of forward and reverse primers designed to amplify one or more fully or partially methylated forms of the modified opposite strand of the DNA region of interest; and
   c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said gene effects the detection of said hybridised probe; and
(iv) qualitatively or quantitatively analysing the detection output of step (iii).

In another aspect there is provided a method of screening for the methylation of a gene or region thereof, said method comprising:
(i) contacting a DNA sample with an agent which modifies unmethylated cytosine residues wherein said sample comprises both the target strand and the opposite strand of said gene target, which DNA sample comprises a low copy number of said DNA region of interest;
(ii) contacting the DNA sample of step (i) with:
   a) a first set of forward and reverse primers designed to amplify one or more fully or partially methylated forms of the modified target strand of the gene target;
   b) a second set of forward and reverse primers designed to amplify one or more fully or partially methylated forms of the modified opposite strand of the gene target; and
   c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said gene effects the detection of said hybridised probe; and
(iv) qualitatively or quantitatively analysing the detection output of step (iii).

In still another aspect, said gene or gene region is a mammalian gene or gene region.

In yet another aspect, said gene is a large intestine neoplasm marker and, more particularly, one or more of:

| | | | |
|---|---|---|---|
| (1) GRASP | (18) ANGPT2 | (35) NKX2-6 | (52) HOXA5 |
| (2) IRX1 | (19) LHX6 | (36) PAX1 | (53) GDNF |
| (3) SOX21 | (20) NEUROD1 | (37) FOXD2 | (54) FAT4 |
| (4) FGF5 | (21) AC149644.1 | (38) SLC6A15 | (55) HOXA2 |
| (5) ZNF471 | (22) CCDC48 | (39) PHC2 | (56) LPHN3 |
| (6) SUSD5 | (23) EVX1 | (40) FLRT2 | (57) ADCYAP1 |
| (7) FOXB1 | (24) GHSR | (41) GATA2 | (58) GRIA2 |
| (8) PDX1 | (25) HSD17B14 | (42) ADCY8 | (59) AQP1 |
| (9) DLX5 | (26) KRBA1 | (43) CNNM1 | (60) BCAT1 |
| (10) ONECUT2 | (27) OTOP1 | (44) IKZF1 | (61) CYP24A1 |
| (11) DMRTA2 | (28) PPYR1 | (45) NKX2-3 | (62) FOXI2 |
| (12) CMTM2 | (29) SRMS | (46) PCDH7 | (63) GSX1 |
| (13) OTX2 | (30) ZNF582 | (47) SNCB | (64) IRF4 |
| (14) LOC145845 | (31) IRX2 | (48) ST8SIA1 | (65) NPY |
| (15) EBF3 | (32) CSMD1 | (49) TRAPPC9 | (66) PDE1B |
| (16) SALL1 | (33) MIR675, | (50) NKX2-2 | (67) CAHM |
| | H19 | | |
| (17) CBX8 | (34) FOXD3 | (51) SLC32A1 | (68) SEPTIN9 |
| (69) BMP3 | (70) NDRG4 | (71) SDC2 | (72) ZCAN18 |
| (73) COL4A2 | (74) FOXF1 | (75) SOX21 | (76) SLC6A15 |
| (77) ST8SIAI | (78) FGF5 | (79) FOXI2 | |

In a further aspect there is provided a method of screening for the methylation of a gene or region thereof, which gene target is selected from the list consisting essentially of:

| | | | |
|---|---|---|---|
| (1) GRASP | (18) ANGPT2 | (35) NKX2-6 | (52) HOXA5 |
| (2) IRX1 | (19) LHX6 | (36) PAX1 | (53) GDNF |
| (3) SOX21 | (20) NEUROD1 | (37) FOXD2 | (54) FAT4 |
| (4) FGF5 | (21) AC149644.1 | (38) SLC6A15 | (55) HOXA2 |
| (5) ZNF471 | (22) CCDC48 | (39) PHC2 | (56) LPHN3 |
| (6) SUSD5 | (23) EVX1 | (40) FLRT2 | (57) ADCYAP1 |
| (7) FOXB1 | (24) GHSR | (41) GATA2 | (58) GRIA2 |
| (8) PDX1 | (25) HSD17B14 | (42) ADCY8 | (59) AQP1 |
| (9) DLX5 | (26) KRBA1 | (43) CNNM1 | (60) BCAT1 |
| (10) ONECUT2 | (27) OTOP1 | (44) IKZF1 | (61) CYP24A1 |
| (11) DMRTA2 | (28) PPYR1 | (45) NKX2-3 | (62) FOXI2 |
| (12) CMTM2 | (29) SRMS | (46) PCDH7 | (63) GSX1 |
| (13) OTX2 | (30) ZNF582 | (47) SNCB | (64) IRF4 |
| (14) LOC145845 | (31) IRX2 | (48) ST8SIA1 | (65) NPY |
| (15) EBF3 | (32) CSMD1 | (49) TRAPPC9 | (66) PDE1B |
| (16) SALL1 | (33) MIR675, | (50) NKX2-2 | (67) CAHM |
| | H19 | | |
| (17) CBX8 | (34) FOXD3 | (51) SLC32A1 | (68) SEPTIN9 |
| (69) BMP3 | (70) NDRG4 | (71) SDC2 | (72) ZCAN18 |
| (73) COL4A2 | (74) FOXF1 | (75) SOX21 | (76) SLC6A15 |
| (77) ST8SIAI | (78) FGF5 | (79) FOXI2 | |

said method comprising:
(i) contacting a DNA sample with an agent which modifies unmethylated cytosine residues wherein said sample comprises both the target strand and the opposite strand of said gene or region thereof, which DNA sample comprises a low copy number of said gene region of interest;
(ii) contacting the DNA sample of step (i) with:
   a) a first set of forward and reverse primers designed to amplify one or more fully or partially methylated forms of the modified target strand of the gene target;
   b) a second set of forward and reverse primers designed to amplify one or more fully or partially methylated forms of the modified opposite strand of the gene target; and
   c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said gene effects the detection of said hybridised probe; and
(iv) qualitatively or quantitatively analysing the detection output of step (iii).

In another aspect there is provided a method of screening for the methylation of a DNA region of interest, said method comprising:
(i) contacting a DNA sample with a bisulphite agent to convert unmethylated cytosine residues to uracil wherein said sample comprises both the target strand and the opposite strand of said DNA region of interest, which DNA sample comprises a low copy number of said gene region of interest;
(ii) contacting the DNA sample of step (i) with:
   a) a first set of forward and reverse primers designed to amplify one or more fully or partially methylated forms of the modified target strand of the DNA region of interest;
   b) a second set of forward and reverse primers designed to amplify one or more fully or partially methylated forms of the modified opposite strand of the DNA region of interest; and
   c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said gene effects the detection of said hybridised probe; and
(iv) qualitatively or quantitatively analysing the detection output of step (iii).

In still another aspect there is provided a method of screening for the methylation of a DNA region of interest, said method comprising:
(i) contacting a DNA sample with a bisulphite agent to convert unmethylated cytosine residues to uracil wherein said sample comprises both the target strand and the opposite strand of said DNA region of interest, which DNA sample comprises a low copy number of said gene region of interest;
(ii) contacting the DNA sample of step (i) with:
   a) a first set of methylation specific forward and reverse primers designed to amplify one or more fully or partially methylated forms of the modified target strand of the DNA region of interest;
   b) a second set of methylation specific forward and reverse primers designed to amplify one or more fully or partially methylated forms of the modified opposite strand of the DNA region of interest; and
   c) optionally one or more probes which incorporate a detection means;
(iii) amplifying the DNA sample of step (ii) wherein if one or more of the probes of step (ii)(c) are used, the extension of said primers along said DNA effects the detection of said hybridised probe; and
(iv) qualitatively or quantitatively analysing the detection output of step (iii).

In one embodiment, said agent that modifies unmethylated cytosine residues is sodium bisulphite.

In one embodiment, said DNA region of interest is a gene target.

In another embodiment, said gene target is selected from the list consisting essentially of:

| | | | |
|---|---|---|---|
| (1) GRASP | (18) ANGPT2 | (35) NKX2-6 | (52) HOXA5 |
| (2) IRX1 | (19) LHX6 | (36) PAX1 | (53) GDNF |
| (3) SOX21 | (20) NEUROD1 | (37) FOXD2 | (54) FAT4 |
| (4) FGF5 | (21) AC149644.1 | (38) SLC6A15 | (55) HOXA2 |
| (5) ZNF471 | (22) CCDC48 | (39) PHC2 | (56) LPHN3 |
| (6) SUSD5 | (23) EVX1 | (40) FLRT2 | (57) ADCYAP1 |
| (7) FOXB1 | (24) GHSR | (41) GATA2 | (58) GRIA2 |
| (8) PDX1 | (25) HSD17B14 | (42) ADCY8 | (59) AQP1 |
| (9) DLX5 | (26) KRBA1 | (43) CNNM1 | (60) BCAT1 |
| (10) ONECUT2 | (27) OTOP1 | (44) IKZF1 | (61) CYP24A1 |
| (11) DMRTA2 | (28) PPYR1 | (45) NKX2-3 | (62) FOXI2 |
| (12) CMTM2 | (29) SRMS | (46) PCDH7 | (63) GSX1 |
| (13) OTX2 | (30) ZNF582 | (47) SNCB | (64) IRF4 |
| (14) LOC145845 | (31) IRX2 | (48) ST8SIA1 | (65) NPY |
| (15) EBF3 | (32) CSMD1 | (49) TRAPPC9 | (66) PDE1B |
| (16) SALL1 | (33) MIR675, | (50) NKX2-2 | (67) CAHM |
| | H19 | | |
| (17) CBX8 | (34) FOXD3 | (51) SLC32A1 | (68) SEPTIN9 |
| (69) BMP3 | (70) NDRG4 | (71) SDC2 | (72) ZCAN18 |
| (73) COL4A2 | (74) FOXF1 | (75) SOX21 | (76) SLC6A15 |
| (77) ST8SIAI | (78) FGF5 | (79) FOXI2 | |

In yet still another embodiment, said gene is one or more of BCAT1, IKZF1, IRF4, GRASP or CAHM, in particular BCAT1 and/or IKZF1.

In still another embodiment, said DNA sample is blood, plasma, serum, saliva, stool, ascites fluid or urine.

In a still further embodiment, said DNA region of interest is ccfDNA, such as disease specific ccfDNA, in particular ctDNA.

In yet another embodiment, said probe is a non-methylation specific probe.

In still yet another embodiment, said probe is a methylation specific probe.

In yet another aspect there is provided a method of screening for the methylation of a DNA region of interest, said method comprising:
(i) contacting a DNA sample with a bisulphite agent to convert unmethylated cytosine residues to uracil wherein said sample comprises both the target strand and the opposite strand of said DNA region of interest which DNA sample comprises a low copy number of said gene region of interest;
(ii) contacting the DNA sample of step (i) with:
   a) a first set of non-methylation specific forward and reverse primers designed to amplify one or more fully or partially methylated forms of the modified target strand of the DNA region of interest;
   b) a second set of non-methylation specific forward and reverse primers designed to amplify one or more fully or partially methylated form of the modified opposite strand of the DNA region of interest; and
   c) one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said DNA effects the detection of said hybridised probe; and
(iv) qualitatively or quantitatively analysing the detection output of step (iii).

In still another embodiment, said agent that modifies unmethylated cytosine residues is sodium bisulphite. In one embodiment, said DNA region of interest is a gene target.

In another embodiment, said gene target is selected from the list consisting essentially of:

| | | | |
|---|---|---|---|
| (1) GRASP | (18) ANGPT2 | (35) NKX2-6 | (52) HOXA5 |
| (2) IRX1 | (19) LHX6 | (36) PAX1 | (53) GDNF |
| (3) SOX21 | (20) NEUROD1 | (37) FOXD2 | (54) FAT4 |
| (4) FGF5 | (21) AC149644.1 | (38) SLC6A15 | (55) HOXA2 |
| (5) ZNF471 | (22) CCDC48 | (39) PHC2 | (56) LPHN3 |
| (6) SUSD5 | (23) EVX1 | (40) FLRT2 | (57) ADCYAP1 |
| (7) FOXB1 | (24) GHSR | (41) GATA2 | (58) GRIA2 |
| (8) PDX1 | (25) HSD17B14 | (42) ADCY8 | (59) AQP1 |
| (9) DLX5 | (26) KRBA1 | (43) CNNM1 | (60) BCAT1 |
| (10) ONECUT2 | (27) OTOP1 | (44) IKZF1 | (61) CYP24A1 |
| (11) DMRTA2 | (28) PPYR1 | (45) NKX2-3 | (62) FOXI2 |
| (12) CMTM2 | (29) SRMS | (46) PCDH7 | (63) GSX1 |
| (13) OTX2 | (30) ZNF582 | (47) SNCB | (64) IRF4 |
| (14) LOC145845 | (31) IRX2 | (48) ST8SIA1 | (65) NPY |
| (15) EBF3 | (32) CSMD1 | (49) TRAPPC9 | (66) PDE1B |
| (16) SALL1 | (33) MIR675, | (50) NKX2-2 | (67) CAHM |
| | H19 | | |
| (17) CBX8 | (34) FOXD3 | (51) SLC32A1 | (68) SEPTIN9 |
| (69) BMP3 | (70) NDRG4 | (71) SDC2 | (72) ZCAN18 |
| (73) COL4A2 | (74) FOXF1 | (75) SOX21 | (76) SLC6A15 |
| (77) ST8SIAI | (78) FGF5 | (79) FOXI2 | |

In yet still another embodiment, said gene is one or more of BCAT1, IKZF1, IRF4, GRASP or CAHM, in particular BCAT1 and/or IKZF1.

In still another embodiment, said DNA sample is blood, plasma, serum, saliva, stool, ascites fluid or urine.

In a still further embodiment, said DNA region of interest is ccfDNA, such as disease specific ccfDNA, in particular ctDNA.

In another further embodiment, said probe is a hydrolysis probe.

In still another aspect, said low copy number is less than 100 copies of target DNA/sample tested. In another embodiment said low copy number is less than 95 copies of target DNA/sample tested, less than 90 copies of target DNA/sample tested, less than 85 copies of target DNA/sample tested, less than 80 copies of target DNA/sample tested, less than 75 copies of target DNA/sample tested, less than 70 copies of target DNA/sample tested, less than 65 copies of target DNA/sample tested, less than 60 copies of target DNA/sample tested, less than 55 copies of target DNA/sample tested, less than 50 copies of target DNA/sample tested, less than 45 copies of target DNA/sample tested, less than 40 copies of target DNA/sample tested, less than 35 copies of target DNA/sample tested, less than 30 copies of target DNA/sample tested, less than 25 copies of target DNA/sample tested, less than 20 copies of target DNA/sample tested, less than 15 copies of target DNA/sample tested, less than 10 copies of target DNA/sample tested or less than 5 copies of target DNA/sample tested. Most particularly said low copy number is less than 50 copies of target DNA/sample tested, still more particularly less than 40 copies of target DNA/sample tested, yet more particularly less than 30 copies of target DNA/sample tested, still more particularly less than 20 copies of target DNA/sample tested. In a further embodiment, said low copy number is less than 10 copies of target DNA/sample tested, most particularly less than 5 copies of target DNA/sample tested.

In yet still another aspect, said amplification is quantitative PCR and said low copy number is the LOD.

In yet another further embodiment said probes are one or more hydrolysis probes directed to a region of partial cytosine methylation wherein said one or more probes collectively hybridise to at least two differing methylation patterns at said region.

In still yet another aspect, said gene is IKZF1 and:

(i) said first set of primers comprise the sequences:

(FWD PRIMER): Chr7 (+); 50, 304, 271-50, 304, 294
SEQ ID NO: 11
5′GACGACGTATTTTTTTCGTGTTTC-3′
(REV PRIMER): Chr7 (-); 50, 304, 350-50, 304, 365
SEQ ID NO: 12
5'-GCGCACCTCTCGACCG-3'

or substantially similar sequences and the probes directed to the amplification product of said first set of primers include one or more of the sequences selected from:

SEQ ID NO: 13
5′-TTTGTATYGGAGTAGYGATTYGGGAGG-3′
SEQ ID NO: 14
5′-TTTGTATCGGAGTAGCGATTCGGGAGG-3′
SEQ ID NO: 15
5′-TTTGTATCGGAGTAGCGATTTGGGAGG-3′
SEQ ID NO: 16
5′-TTTGTATCGGAGTAGTGATTCGGGAGG-3′
SEQ ID NO: 17
5′-TTTGTATTGGAGTAGCGATTCGGGAGG-3′
SEQ ID NO: 18
5′-TTTGTATCGGAGTAGTGATTTGGGAGG-3′
SEQ ID NO: 19
5′-TTTGTATTGGAGTAGCGATTTGGGAGG-3′
SEQ ID NO :20
5′-TTTGTATTGGAGTAGTGATTCGGGAGG-3′
SEQ ID NO: 21
5′-TTTGTATTGGAGTAGTGATTTGGGAGG-3′

or substantially similar sequences; and

(ii) said second set of primers comprise the sequences:

(FWD PRIMER): Chr7 (−); 50, 304, 295-50, 304, 314
SEQ ID NO: 77
5′-CGCGTAGAAGGGCGTAGAGC-3′
(REV PRIMER): Chr7 (+); 50, 304, 234-50, 304, 254
SEQ ID NO: 78
5′-GCGCGAACCGAAAAACTCGAC-3′

or substantially similar sequences and the probes directed to the amplification product of said second set of primers include one or more of the sequences selected from:

SEQ ID NO: 79
5′-AAYGAYGCACCCTCTCYGTATCCY-3′
SEQ ID NO: 80
5′-AACGACGCACCCTCTCCGTATCCT-3′
SEQ ID NO: 81
5′-AATGACGCACCCTCTCCGTATCCC-3′
SEQ ID NO: 82
5′-AACGATGCACCCTCTCCGTATCCC-3′
SEQ ID NO: 83
5′-AACGACGCACCCTCTCTGTATCCC-3′
SEQ ID NO: 84
5′-AATGATGCACCCTCTCCGTATCCC-3′
SEQ ID NO: 85
5′-AATGACGCACCCTCTCTGTATCCC-3′
SEQ ID NO: 86
5′-AATGACGCACCCTCTCCGTATCCT-3′
SEQ ID NO: 87
5′-AACGATGCACCCTCTCTGTATCCC-3′
SEQ ID NO: 88
5′-AACGATGCACCCTCTCCGTATCCT-3′
SEQ ID NO: 89
5′-AACGACGCACCCTCTCTGTATCCT-3′
SEQ ID NO: 90
5′-AATGATGCACCCTCTCTGTATCCC-3′
SEQ ID NO: 91
5′-AATGACGCACCCTCTCTGTATCCT-3′
SEQ ID NO: 92
5′-AACGATGCACCCTCTCTGTATCCT-3′
SEQ ID NO: 93
5′-AATGATGCACCCTCTCTGTATCCT-3′
SEQ ID NO: 94
5′-AACGACGCACCCTCTCCGTATCCC-3′
SEQ ID NO: 95
5′-AATGATGCACCCTCTCCGTATCCT-3′

or substantially similar sequences.

In yet still another aspect, said gene is IKZF1 and:

(i) said first set of primers comprise the sequences:

(FWD PRIMER): Chr7 (+); 50, 304, 271-50, 304, 294
SEQ ID NO: 11
5′ GACGACGTATTTTTTTCGTGTTTC-3′
(REV PRIMER): Chr7 (−); 50, 304, 350-50, 304, 365
SEQ ID NO:12
5′-GCGCACCTCTCGACCG-3′

or substantially similar sequences and the probes directed to the amplification product of said first set of primers include one or more of the sequences selected from:

SEQ ID NO: 13
5′-TTTGTATYGGAGTAGYGATTYGGGAGG-3′
SEQ ID NO: 14
5′-TTTGTATCGGAGTAGCGATTCGGGAGG-3′
SEQ ID NO: 15
5′-TTTGTATCGGAGTAGCGATTTGGGAGG-3′
SEQ ID NO: 16
5′-TTTGTATCGGAGTAGTGATTCGGGAGG-3′
SEQ ID NO: 17
5′-TTTGTATTGGAGTAGCGATTCGGGAGG-3′
SEQ ID NO: 18
5′-TTTGTATCGGAGTAGTGATTTGGGAGG-3′
SEQ ID NO: 19
5′-TTTGTATTGGAGTAGCGATTTGGGAGG-3′
SEQ ID NO: 20
5′-TTTGTATTGGAGTAGTGATTCGGGAGG-3′
SEQ ID NO: 21
5′-TTTGTATTGGAGTAGTGATTTGGGAGG-3′

or substantially similar sequences; and

(ii) said second set of primers comprise the sequences:

(FWD PRIMER): Chr7 (−); 50, 304, 366-50, 304, 391
SEQ ID NO: 22
5′TTGTTTCGTAGTCGGTTCGGTTTCG 3′
(REV PRIMER): Chr7 (+); 50, 304, 271-50, 304, 294
SEQ ID NO: 23
5′-AACGACGCACCCTCTCCGTATCCC-3'

or substantially similar sequences and the probes directed to the amplification product of said second set of primers include one or more of the sequences selected from:

SEQ ID NO: 24
5′-TTTTTYGGATYGTTGTTTYGGTATAGG-3′
SEQ ID NO: 25
5′-TTTTTCGGATCGTTGTTTCGGTATAGG-3′
SEQ ID NO: 26
5′-TTTTTCGGATCGTTGTTTTGGTATAGG-3′
SEQ ID NO: 27
5′-TTTTTCGGATTGTTGTTTCGGTATAGG-3′
SEQ ID NO: 28
5′-TTTTTTGGATCGTTGTTTCGGTATAGG-3′
SEQ ID NO: 29
5′-TTTTTCGGATTGTTGTTTTGGTATAGG-3′
SEQ ID NO: 30
5′-TTTTTTGGATTGTTGTTTCGGTATAGG-3′
SEQ ID NO: 31
5′-TTTTTTGGATCGTTGTTTTGGTATAGG-3′
SEQ ID NO: 32
5′-TTTTTTGGATTGTTGTTTTGGTATAGG-3′

or substantially similar sequences.

In another further aspect, said gene is IKZF1 and:

(i) said first set of primers comprise the sequences:

(FWD PRIMER): Chr7 (+); 50,304,271-50,304,294
SEQ ID NO: 11
5′ GACGACGTATTTTTTTCGTGTTTC-3′
(REV PRIMER): Chr7 (−); 50,304,350-50,304,365
SEQ ID NO: 12
5′-GCGCACCTCTCGACCG-3′

or substantially similar sequences and the probes directed to the amplification product of said first set of primers include one or more of the sequences selected from:

SEQ ID NO: 13
5′-TTTGTATYGGAGTAGYGATTYGGGAGG-3′
SEQ ID NO: 14
5′-TTTGTATCGGAGTAGCGATTCGGGAGG-3′
SEQ ID NO: 15
5′-TTTGTATCGGAGTAGCGATTTGGGAGG-3′
SEQ ID NO: 16
5′-TTTGTATCGGAGTAGTGATTCGGGAGG-3′
SEQ ID NO: 17
5′-TTTGTATTGGAGTAGCGATTCGGGAGG-3′
SEQ ID NO: 18
5′-TTTGTATCGGAGTAGTGATTTGGGAGG-3′
SEQ ID NO: 19
5′-TTTGTATTGGAGTAGCGATTTGGGAGG-3′
SEQ ID NO: 20
5′-TTTGTATTGGAGTAGTGATTCGGGAGG-3′
SEQ ID NO: 21
5′-TTTGTATTGGAGTAGTGATTTGGGAGG-3′

or substantially similar sequences; and

(ii) said second set of primers comprise the sequences:

(FWD PRIMER): Chr7 (−); 50,304,329-50,304,355
SEQ ID NO: 33
5′-CGGTCGTTTTTCGGATCGTTGTTTCGG-3′
(REV PRIMER): Chr7 (+); 50,304,271-50,304,294
SEQ ID NO: 23
5′-AACGACGCACCCTCTCCGTATCCC-3′

or substantially similar sequences and the probes directed to the amplification product of said second set of primers include one or more of the sequences selected from:

SEQ ID NO: 34
5′-YGYGTAGAAGGGYGTAGAGYG-3′
SEQ ID NO: 35
5′-CGCGTAGAAGGGCGTAGAGCG-3′
SEQ ID NO: 36
5′-CGCGTAGAAGGGCGTAGAGTG-3′
SEQ ID NO: 37
5′-CGCGTAGAAGGGTGTAGAGTG-3′
SEQ ID NO: 38
5′-CGTGTAGAAGGGTGTAGAGTG-3′
SEQ ID NO: 39
5′-TGTGTAGAAGGGTGTAGAGTG-3′
SEQ ID NO: 40
5′-TGCGTAGAAGGGCGTAGAGCG-3′
SEQ ID NO: 41
5′-CGTGTAGAAGGGCGTAGAGCG-3′
SEQ ID NO: 42
5′-CGCGTAGAAGGGTGTAGAGCG-3′
SEQ ID NO: 43
5′-TGTGTAGAAGGGCGTAGAGCG-3′
SEQ ID NO: 44
5′-TGCGTAGAAGGGTGTAGAGCG-3′
SEQ ID NO: 45
5′-TGCGTAGAAGGGCGTAGAGTG-3′
SEQ ID NO: 46
5′-TGTGTAGAAGGGTGTAGAGCG-3′
SEQ ID NO: 47
5′-TGTGTAGAAGGGCGTAGAGTG-3′
SEQ ID NO: 48
5′-TGCGTAGAAGGGTGTAGAGTG-3′
SEQ ID NO: 49
5′-CGTGTAGAAGGGCGTAGAGTG-3′
SEQ ID NO: 50
5′-CGTGTAGAAGGGTGTAGAGCG-3′

or substantially similar sequences.

In a further aspect, said gene is BCAT1 and:

(i) said first set of primers comprise the sequences:

(FWD PRIMER): chr12 (+); 24,949,138-24,949,164
SEQ ID NO: 97
5′-TTAGTGTTTTTTTGTTGATGTAATTCG-3′
(REV PRIMER): chr12 (−); 24,949,058-24,949,074
SEQ ID NO: 65
5′-CAATACCCGAAACGACGACG-3′

or substantially similar sequences and the probes directed to the amplification product of said first set of primers include the sequence:

SEQ ID NO: 66
5′-TTCGTCGCGAGAGGGTCGGTT-3′

or substantially similar sequences; and

(ii) said second set of primers comprise the sequences:

(FWD PRIMER): chr12 (−); 24,949,058-24,949,082
SEQ ID NO: 96
5′-TAGTGTTCGAGGCGGCGGCGAGTAT-3′
(REV PRIMER): chr12 (+); 24,949,140-24,949,159
SEQ ID NO: 62
5′-ATCTTCCTACTAATACAATCCGCTAAATC-3′

or substantially similar sequences and the probes directed to the amplification product of said second set of primers include the sequence:

SEQ ID NO: 63
5′-GATCGGTTTTTTCGCGGCGGA-3′

or substantially similar sequence.

In a still further aspect, said gene is BCAT1 and:

(i) said first set of primers comprise the sequences:

(FWD PRIMER): chr12 (+); 24,949,131-24,949,159
SEQ ID NO: 64
5′-GTTTTTTTGTTGATGTAATTCGTTAGGTC-3′
(REV PRIMER): chr12 (−); 24,949,058-24,949,074
SEQ ID NO: 65
5′-CAATACCCGAAACGACGACG-3′

or substantially similar sequences and the probes directed to the amplification product of said first set of primers include the sequence:

SEQ ID NO: 66
5′-TTCGTCGCGAGAGGGTCGGTT-3′

or substantially similar sequences; and

(ii) said second set of primers comprise the sequences:

(FWD PRIMER): chr12 (−); 24,949,058-24,949,085
SEQ ID NO: 61
5′-TAGTGTTCGAGGCGGCGGCGAGTATACG-3′
(REV PRIMER): chr12 (+); 24,949,140-24,949,159
SEQ ID NO: 62
5′-ATCTTCCTACTAATACAATCCGCTAAATC-3′

or substantially similar sequences and the probes directed to the amplification product of said second set of primers include the sequence:

SEQ ID NO: 63
5′-GATCGGTTTTTTCGCGGCGGA-3′

or substantially similar sequence.

In another aspect, said gene is IRF4 and:

(i) said first set of primers comprise the sequences:

(FWD PRIMER): Chr6 (+): 391,546-391,564
SEQ ID NO: 108
5′-GGCGCGGGAATTTTATTTC-3′
(REV PRIMER): Chr6 (−): 391,605-391,622
SEQ ID NO: 109
5′-AAACCGACCGAACGAACG-3′

or substantially similar sequences and the probes directed to the amplification product of said first set of primers include the sequence:

SEQ ID NO: 110
5′-AAAACCGACGTAAAAACTAAAAACTACCGCGA-3′

or substantially similar sequences; and

(ii) said second set of primers comprise the sequences:

(FWD PRIMER): Chr6 (−): 391,614-391,636
SEQ ID NO: 111
5′-TAAGTCGAGAGTCGGGGTCGGTC-3′
(REV PRIMER): Chr6 (+): 391,524-391,539
SEQ ID NO: 112
5′-CGACGCGCGAAAAATC-3′

or substantially similar sequences and the probes directed to the amplification product of said second set of primers include one or more of the sequences selected from:

SEQ ID NO: 113
5′-CCAACCTTCACGCCGACCCTAAAACTCG-3′
SEQ ID NO: 115
5′-CCTTCACGCCGACCCTAAAACTCG-3′

or substantially similar sequence.

In still another aspect, said gene is IRF4 and:

(i) said first set of primers comprise the sequences:

(FWD PRIMER): Chr6 (+): 391,546-391,564
SEQ ID NO: 108
5′-GGCGCGGGAATTTTATTTC-3′
(REV PRIMER): Chr6 (−): 391,605-391,622
SEQ ID NO: 109
5′-AAACCGACCGAACGAACG-3′

or substantially similar sequences and the probes directed to the amplification product of said first set of primers include the sequence:

SEQ ID NO: 110
5′-AAAACCGACGTAAAAACTAAAAACTACCGCGA-3′

or substantially similar sequences; and

(ii) said second set of primers comprise the sequences:

(FWD PRIMER): Chr6 (−): 391,614-391,630
SEQ ID NO: 114
5′-GAGAGTCGGGGTCGGTC-3′
(REV PRIMER): Chr6 (+): 391,524-391,539
SEQ ID NO: 12
5′-CGACGCGCGAAAAATC-3′

or substantially similar sequences and the probes directed to the amplification product of said second set of primers include one or more of the sequences selected from:

SEQ ID NO: 113
5′-CCAACCTTCACGCCGACCCTAAAACTCG-3′
SEQ ID NO: 115
5′-CCTTCACGCCGACCCTAAAACTCG-3′

or substantially similar sequence.

In yet another aspect, said gene is IRF4 and:

(i) said first set of primers comprise the sequences:

(FWD PRIMER): Chr6 (+): 391,546-391,564
SEQ ID NO: 108
5′-GGCGCGGGAATTTTATTTC-3′
(REV PRIMER): Chr6 (−): 391,605-391,622
SEQ ID NO: 109
5′-AAACCGACCGAACGAACG-3″

or substantially similar sequences and the probes directed to the amplification product of said first set of primers include the sequence:

SEQ ID NO: 110
5′-AAAACCGACGTAAAAACTAAAAACTACCGCGA-3′

or substantially similar sequences; and

(ii) said second set of primers comprise the sequences:

(FWD PRIMER): Chr6 (−): 391,624-391,649
SEQ ID NO: 116
5′-GAGAGGGATTTTGTAAGTCGAGAGTC-3′
(REV PRIMER): Chr6 (+): 391,524-391,539
SEQ ID NO: 112
5′-CGACGCGCGAAAAATC-3′

or substantially similar sequences and the probes directed to the amplification product of said second set of primers include one or more of the sequences selected from:

SEQ ID NO: 113
5′-CCAACCTTCACGCCGACCCTAAAACTCG-3′
SEQ ID NO: 115
5′-CCTTCACGCCGACCCTAAAACTCG-3′

or substantially similar sequence.

Another aspect of the present invention is directed to a method of diagnosing or monitoring a condition in a patient, which condition is characterised by modulation of the methylation of a DNA region of interest, said method comprising:
(i) contacting a DNA sample with an agent which modifies unmethylated cytosine residues wherein said sample comprises both the target strand and the opposite strand of said DNA region of interest which DNA sample comprises a low copy number of said gene region of interest;
(ii) contacting the DNA sample of step (i) with:
   a) a first set of forward and reverse primers designed to amplify one or more fully or partially methylated forms of the modified target strand of the DNA region of interest;
   b) a second set of forward and reverse primers designed to amplify one or more fully or partially methylated forms of the modified opposite strand of the DNA region of interest; and
   c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said gene effects the detection of said hybridised probe; and
(iv) qualitatively or quantitatively analysing the detection output of step (iii).

Still another aspect of the present invention is directed a kit for assaying biological samples comprising one or more primers and/or probes for detecting one or more neoplastic markers in accordance with the method of the present invention and reagents useful for facilitating the detection by said primers and/or probes. Further means may also be included, for example, to receive a biological sample.

In a further aspect there is provided a kit for screening for the methylation of a DNA region of interest, said kit comprising:
a) a first set of forward and reverse primers designed to amplify one or more fully or partially methylated forms of a target strand of the DNA region of interest, which primers are designed to hybridise to a form of the DNA region of interest which has undergone modification by an agent of unmethylated cytosines;
b) a second set of forward and reverse primers designed to amplify one or more fully or partially methylated forms of the modified opposite strand of the DNA region of interest; and
c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means.

### BRIEF DESCRIPTION OF THE DRAWINGS

FIG. 1 is a schematic representation showing how the bisulphite treatment results in non-complementary single DNA strands. A) Treatment of DNA with a bisulphite solution converts unmethylated cytosine (C) residues to uracil (U) residues whilst methylated cytosine (mC) residues are unaffected. Consequently, the resulting bisulphite treated DNA strands are no longer complementary. Uracils are replaced by the DNA analog thymine (T) during the subsequent PCR amplification. This allows for differentiation of methylated and unmethylated cytosines. B) PCR amplification of wildtype DNA using oligonucleotides (FWD, REV) complementary to top and bottom strands results in a doubling of DNA material after 1 cycle. C) Unless two oligonucleotide primer sets (bisulphite converted and methylated and/or unmethylated specific), only the target strand of the original wildtype DNA will be amplified.

FIG. 2 is a graphical representation of real-time PCR amplification curves for targeted methylation regions in A) BCAT1 and B) IKZF1 using bisulphite and methylation specific oligonucleotides (primers/probes) detecting respective target regions in BCAT1 and IKZF1 (grey) or both target regions and opposite target regions (red) of 2000 pg of fully methylated and bisulphite converted DNA. A ΔCt value of 1 indicates that twice the amount of template is being amplified.

FIG. 3 is a graphical representation of Digital Droplet PCR based quantification of targeted methylation regions in ACTB, BCAT1 and IKZF1 using oligonucleotides (primers/probes annealing to A) target regions or B) target and opposite-target regions in 2000 pg bisulphite treated fully methylated DNA (equivalent to −606 genomic copies). Fluorescent populations are shown for the target strand of ACTB, BCAT1 and IKZF1 as well as the opposite target strand for BCAT1 and IKZF1 in B), denoted as BCAT1′ and IKZF1′. Numbers in brackets refer to the determined copies of target(s), copies/well.

FIG. 4 is a graphical representation of pooled human plasma spiked with various amounts of fully methylated DNA (range 0-300 pg/mL). Circulating cell-free DNA was subsequently extracted and bisulphite converted. Between 5 and 13 sample replicates of the resulting DNA were analysed in triplicates using the assays described in FIG. 3. A sample was deemed positive if any of the three PCR replicate was positive for BCAT1 and/or IKZF1. The limit of detection (LOD) was calculated using Probit analysis.

FIG. 5 is a graphical representation of PCR replicate positivity measured in pooled human plasma spiked with various amounts of fully methylated DNA (range 0-500 pg/mL). Circulating cell-free DNA was subsequently extracted and bisulphite converted. The resulting DNA were analysed using bisulphite and methylation specific oligonucleotides (primers/probes) detecting respective target regions in IKZF1 and BCAT1 (red bars; SEQ IDs: 11-21, 64-66) or both target regions and opposite target regions (black bars; SEQ IDs: 11-21, 77-95, 62-63, 96, 65-66, 97). A sample was deemed positive if any of the three PCR replicates was positive for BCAT1 and/or IKZF1.

FIG. 6 is a graphical representation of qPCR based positivity of methylation regions in BCAT1 and IKZF1 on target regions (red) or both target and opposite target regions (black), using the assays described in FIG. 5. In each case, numerous replicates (11270) containing 3 pg (equivalent to −1 genomic copy) of sonicated, bisulphite treated, fully methylated DNA per sample were amplified and the level of positivity across all samples was determined. A sample was deemed positive if any of the three PCR replicates was positive for BCAT1 and/or IKZF1.

FIG. 7 is a graphical representation of real-time PCR amplification curves for targeted methylation regions in IRF4 using bisulphite and methylation specific oligonucleotides (primers/probes) detecting respective target region in IRF4 (black; SEQ IDs: 108-110) and both target and opposite target region (red; SEQ IDs: 108-110 and 111-113) of 2000 pg of fully methylated and bisulphite converted DNA. A ΔCt value of 1 indicates that twice the amount of template is being amplified.

FIG. 8 details the IKZF1, BCAT1, IRF4 and ACTB sequences used in the Examples.

### DETAILED DESCRIPTION OF THE INVENTION

The present invention is predicated, in part, on the determination that the reliability and sensitivity of PCR-based methylation analysis of DNA can be significantly improved if the amplification reaction is designed to use at least two sets of primers (and probes if a quantitative analysis is to be performed) wherein at least one primer set is designed to hybridise to and amplify the target strand of a bisulphite converted DNA region of interest and the other primer set is designed to hybridise to and amplify the opposite strand of a bisulphite converted DNA region of interest. The development of this method has been necessitated due to the elucidation of the true extent of the unreliability and lack of sensitivity of amplification based analysis directed to low copy number DNA methylation targets, such as methylation markers of circulating cell free DNA (ccfDNA), for example disease-specific ccfDNA, in particular circulating tumour DNA (ctDNA). Specifically, in addition to the fact that bisulphite conversion of a double stranded DNA region of interest leads to the generation of two non-complementary strands, which are therefore not capable of both being amplified (in the usual way that a PCR reaction proceeds for untreated DNA) by a forward and reverse primer set designed on the basis of one of the bisulphite converted DNA strands, the bisulphite conversion step itself causes DNA degradation due to the high pH and temperature required to drive the conversion process. However, it has still further been determined that bisulphite treatment may also cause random nicking (destruction of the DNA phosphate backbone) of both strands of the DNA molecule. Where such nicking occurs in the targeted amplicon region, the DNA becomes non-amplifiable.

Accordingly, the fact that only one strand can be amplified using prior art methods of quantitatively or qualitatively conducting bisulphite conversion-based DNA methylation analysis, and the further overall DNA degradation induced by the harsh conditions of the bisulphite conversion process, significantly reduces the overall DNA copy number available for initial amplification, thereby leading to a significant reduction in sensitivity—potentially to the point of being unable to accurately detect gene hypermethylation in a sample of interest. However, it has now been still further determined that the occurrence of the random nicking not only potentially further reduces the concentration of available starting material but, most critically, leads to unreliable and skewed results which are characterized by an unacceptable level of false negative results, this being highly undesirable in the context of disease diagnosis and screening. Whereas false positive results are also undesirable, such patients will at least likely undergo further testing. However, a false negative result may lead to a patient being incorrectly classed as disease free—with potentially fatal consequences.

The determination of the extent of the severity of the impact of the random nicking of bisulphite converted DNA to the accuracy of the amplification results in low copy number DNA which are obtained thereafter was unexpected and has necessitated the redesigning and development of new amplification methodology which, in addition to significantly improving the accuracy of the amplification results which are obtained, also improves the overall sensitivity of the screening test. Prior art methods have focussed efforts to improve efficiency on optimising the bisulphite conversion step, which has led to even higher levels of degradation of the starting DNA population, this being a particular problem where very low starting copy numbers of the DNA of interest are present. However, the present inventors have designed a method such that where one is amplifying a target input population which is in low abundance, not only is there an improvement in sensitivity, but the copy number which is obtained represents a doubling of the copy number which would theoretically be obtained if 100% of that same input target population was amplified from the target strand alone. This has been achieved by designing the amplification assay to incorporate the use of at least two distinct sets of primers and probes which are designed to enable the amplification of both the target and the opposite strand of the methylated DNA region if interest. However, although one might logically expect that this “doubling” would be calculated relative to the amount of amplifiable DNA present after the bisulphite conversion step, which has now been determined to degrade and/or nick significantly more DNA than was previously thought to occur, in the context of the present invention, it has been very unexpectedly found that the “doubling” of copy number which is observed is actually a doubling of the level of pre-bisulphite treated DNA, that is the level which exists prior to the significant nicking and degradation which is known to be induced by bisulphite conversion. The development of the method of the present invention has therefore now enabled the routine application of methylation specific amplification assays which exhibit significantly higher sensitivity and accuracy than has been previously attainable. In the context of cancer diagnosis, false negative results can have extremely serious consequences for a patient. Accordingly, the method of the present invention provides a simple but robust means of ensuring a high level of sensitivity when assessing DNA methylation.

Accordingly, one aspect of the present invention is directed to a method of screening for the methylation of a DNA region of interest, said method comprising:
(i) contacting a DNA sample with an agent which modifies unmethylated cytosine residues wherein said sample comprises both the target strand and the opposite strand of said DNA region of interest;
(ii) contacting the DNA sample of step (i) with:
   a) a first set of forward and reverse primers designed to amplify one or more fully or partially methylated forms of the modified target strand of the DNA region of interest;
   b) a second set of forward and reverse primers designed to amplify one or more fully or partially methylated forms of the modified opposite strand of the DNA region of interest; and
   c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said gene effects the detection of said hybridised probe; and
(iv) qualitatively or quantitatively analysing the detection output of step (iii) to obtain a result which exhibits a reduced incidence of false negative results.

More particularly there is provided an improved method of screening for the methylation of a DNA region of interest, said method comprising:
(i) contacting a DNA sample with an agent which modifies unmethylated cytosine residues wherein said sample comprises both the target strand and the opposite strand of said DNA region of interest, which DNA sample comprises a low copy number of said DNA region of interest;
(ii) contacting the DNA sample of step (i) with:
   a) a first set of forward and reverse primers designed to amplify one or more fully or partially methylated forms of the modified target strand of the DNA region of interest;
   b) a second set of forward and reverse primers designed to amplify one or more fully or partially methylated forms of the modified opposite strand of the DNA region of interest; and
   c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said gene effects the detection of said hybridised probe; and
(iv) qualitatively or quantitatively analysing the detection output of step (iii).

In one embodiment, the result which is obtained from step (iv) exhibits a reduced incidence of false negative results.

Reference to a “DNA region of interest” should be understood as a reference to any region or form of DNA, including one or more CpG sites (“islands”) the methylation status of which are sought to be analysed. This may be, for example, a gene, part of a gene, an intergenic region or a promoter. To this end, reference to “gene” should be understood as a reference to a DNA molecule that codes for a protein product, whether that be a full length protein or a protein fragment. It should be understood, however, that there are some genes that have been identified which are not known to necessarily produce a protein product and may only be transcribed to RNA. Reference to “gene” herein should therefore be understood to include reference to both types of genes. In terms of genomic DNA or the RNA transcribed therefrom, the gene will generally be expected to include both intronic and exonic regions. The subject nucleic acid region of interest may also be a portion of genomic DNA which is not known to be associated with any specific gene (such as the commonly termed “junk” DNA regions). The nucleic acid target of interest may also be any region of genomic DNA produced by recombination, either between two regions of genomic DNA or one region of genomic DNA and a region of foreign DNA such as a virus or an introduced sequence. The DNA that is the subject of analysis need not necessarily be genomic DNA, although it is generally understood that recombinantly expressed DNA, such as cDNA, is often not methylated. Nevertheless, the present invention should be understood to extend to the analysis of any source or form of DNA which may be methylated.

Without limiting the present invention in any way, DNA methylation is universal in bacteria, plants, and animals. DNA methylation is a type of chemical modification of DNA that is stable over rounds of cell division but does not involve changes in the underlying DNA sequence of the organism. Chromatin and DNA modifications are two important features of epigenetics and play a role in the process of cellular differentiation, allowing cells to stably maintain different characteristics despite containing the same genomic material. In eukaryotic organism's DNA methylation occurs only at the number 5 carbon of the cytosine pyrimidine ring. In mammals, DNA methylation occurs mostly at the number 5 carbon of the cytosine of a CpG dinucleotide. CpG dinucleotides comprise approximately 1% of the human genome.

70-80% of all CpGs are methylated in mammals. CpGs may be grouped in clusters called “CpG islands” that are typically present in the 5′-end of regulatory regions of many genes. In many disease processes such as cancer, gene promoters and/or CpG islands acquire aberrant methylation. Hypomethylation is often seen in oncogenes whereas aberrant DNA hypermethylation is often associated with heritable transcriptional silencing of tumour suppressor genes. DNA methylation may impact the transcription of genes in two ways. First, the methylation of DNA may itself physically impede the binding of transcriptional factors to the gene, thus blocking transcription. Second, methylated DNA may be bound by proteins known as Methyl-CpG-binding domain proteins (MBDs). MBD proteins then recruit additional proteins to the locus, such as histone deacetylases and other chromatin remodelling proteins that can modify histones, thereby forming compact, inactive chromatin termed silent chromatin. This link between DNA methylation and chromatin structure is very important. In particular, loss of Methyl-CpG-binding Protein 2 (MeCP2) has been implicated in Rett syndrome and Methyl-CpG binding domain protein 2 (MBD2) mediates the transcriptional silencing of hypermethylated genes in cancer.

In humans, the process of DNA methylation is carried out by three enzymes, DNA methyltransferase 1, 3a and 3b (DNMT1, DNMT3a, DNMT3b). It is thought that DNMT3a and DNMT3b are the de novo methyltransferases that set up DNA methylation patterns early in development. DNMT1 is the proposed maintenance methyltransferase that is responsible for copying DNA methylation patterns to the daughter strands during DNA replication. DNMT3L is a protein that is homologous to the other DNMT3s but has no catalytic activity. Instead, DNMT3L assists the de novo methyltransferases by increasing their ability to bind to DNA and stimulating their activity. Finally, DNMT2 has been identified as an “enigmatic” DNA methyltransferase homolog, containing all 10 sequence motifs common to all DNA methyltransferases; however, DNMT2 may not methylate DNA but instead has been shown to methylate a small RNA.

The term “methylation” should therefore be understood to mean the presence of a methyl group added by the action of a DNA methyl transferase enzyme to cytosine or adenosine bases in a region of nucleic acid, e.g. genomic DNA. In this regard, the general reference to detecting fully or partially methylated forms of DNA should be understood to include the detection of hemimethylated DNA.

In one embodiment, said nucleic acid target of interest is a DNA gene or gene region; such as the promoter region. Reference to “gene target” should therefore be understood as a reference to a gene or region of a gene in respect of which the methylation is to be interrogated. As would be understood by the person of skill in the art, the reference to “gene” includes the promoter region of the gene.

Reference to a “region of a gene” or “gene region” should be understood as a reference to any stretch of DNA which corresponds to part of a gene but not the entire gene. For example, the DNA which is analysed by the method of a present invention may be fragmented, such as during in vivo circulation due to the action of DNAse, during its isolation, or it may have been cleaved as a preliminary step prior to analysis by the method of the present invention.

According to this embodiment there is provided a method of screening for the methylation of a gene or region thereof, said method comprising:
(i) contacting a DNA sample with an agent which modifies unmethylated cytosine residues wherein said sample comprises both the target strand and the opposite strand of said gene target, which DNA sample comprises a low copy number of said gene region of interest;
(ii) contacting the DNA sample of step (i) with:
   a) a first set of forward and reverse primers designed to amplify one or more fully or partially methylated forms of the modified target strand of the gene target;
   b) a second set of forward and reverse primers designed to amplify one or more fully or partially methylated forms of the modified opposite strand of the gene target; and
   c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said gene effects the detection of said hybridised probe; and
(iv) qualitatively or quantitatively analysing the detection output of step (iii).

In another embodiment, said gene or gene region is a mammalian gene or gene region.

In a further embodiment, said gene is a large intestine neoplasm marker and, more particularly, one or more of:

| | | | |
|---|---|---|---|
| (1) GRASP | (18) ANGPT2 | (35) NKX2-6 | (52) HOXA5 |
| (2) IRX1 | (19) LHX6 | (36) PAX1 | (53) GDNF |
| (3) SOX21 | (20) NEUROD1 | (37) FOXD2 | (54) FAT4 |
| (4) FGF5 | (21) AC149644.1 | (38) SLC6A15 | (55) HOXA2 |
| (5) ZNF471 | (22) CCDC48 | (39) PHC2 | (56) LPHN3 |
| (6) SUSD5 | (23) EVX1 | (40) FLRT2 | (57) ADCYAP1 |
| (7) FOXB1 | (24) GHSR | (41) GATA2 | (58) GRIA2 |
| (8) PDX1 | (25) HSD17B14 | (42) ADCY8 | (59) AQP1 |
| (9) DLX5 | (26) KRBA1 | (43) CNNM1 | (60) BCAT1 |
| (10) ONECUT2 | (27) OTOP1 | (44) IKZF1 | (61) CYP24A1 |
| (11) DMRTA2 | (28) PPYR1 | (45) NKX2-3 | (62) FOXI2 |
| (12) CMTM2 | (29) SRMS | (46) PCDH7 | (63) GSX1 |
| (13) OTX2 | (30) ZNF582 | (47) SNCB | (64) IRF4 |
| (14) LOC145845 | (31) IRX2 | (48) ST8S1A1 | (65) NPY |
| (15) EBF3 | (32) CSMD1 | (49) TRAPPC9 | (66) PDE1B |
| (16) SALL1 | (33) MIR675, | (50) NKX2-2 | (67) CAHM |
| | H19 | | |
| (17) CBX8 | (34) FOXD3 | (51) SLC32A1 | (68) SEPTIN9 |
| (69) BMP3 | (70) NDRG4 | (71) SDC2 | (72) ZCAN18 |
| (73) COL4A2 | (74) FOXF1 | (75) SOX21 | (76) SLC6A15 |
| (77) ST8SIA1 | (78) FGF5 | (79) FOXI2 | |

These genes are specified herein by reference to both gene name and a set of human chromosomal coordinates. Both the gene names and the chromosomal coordinates would be well known to, and understood by, the person of skill in the art. In general, a gene can be routinely identified by reference to its name, via which both its sequences and chromosomal location can be routinely obtained, or by reference to its chromosomal coordinates, via which both the gene name and its sequence can also be routinely obtained.

Reference to “genes” should be understood as a reference to all forms of these molecules and to fragments or variants thereof. As would be appreciated by the person skilled in the art, some genes are known to exhibit allelic variation between individuals or single nucleotide polymorphisms. Such variations include SNPs, insertions and deletions of varying size and simple sequence repeats, such as dinucleotide and trinucleotide repeats. Variants include nucleic acid sequences from the same region sharing at least 90%, 95%, 98%, 99% or greater sequence identity i.e. having one or more deletions, additions, substitutions, inverted sequences etc. relative to the genes described herein. Accordingly, the present invention should be understood to extend to such variants which, in terms of the present diagnostic applications, achieve the same outcome despite the fact that minor genetic variations between the actual nucleic acid sequences may exist between individuals. The present invention should therefore be understood to extend to all forms of DNA that arise from any other mutation, polymorphic or allelic variation.

The GRCh38/hg38 chromosomal coordinates corresponding to the genes detailed above are as follows:

| | |
|---|---|
| (1) GRASP | chr12: 52006945-52015889 |
| (2) IRX1 | chr5: 3596054-3601403 |
| (3) SOX21 | chr13: 94709625-94712543 |
| (4) FGF5 | chr4: 80266588-80291017 |
| (5) ZNF471 | chr19: 56507843-56530214 |
| (6) SUSD5 | chr3: 33150045-33219215 |
| (7) FOXB1 | chr15: 60004222-60005943 |
| (8) PDX1 | chr13: 27920031-27926314 |
| (9) DLX5 | chr7: 97020390-97024831 |
| (10) ONECUT2 | chr18: 57435685-57491298 |
| (11) DMRTA2 | chr1: 50417551-50423447 |
| (12) CMTM2 | chr18: 57435685-57491298 |
| (13) OTX2 | chr14: 56,799,905-56,810,469 |
| (14) LOC145845 | chr15: 36864443-36886533 |
| (15) EBF3 | chr10: 129835232-129963827 |
| (16) SALL1 | chr16: 51135975-51151272 |
| (17) CBX8 | chr17: 79794377-79797116 |
| (69) BMP3 | chr4: 81030965-81057625 |
| (18) ANGPT2 | chr8: 6499651-6563263 |
| (19) LHX6 | chr9: 122202577-122221740 |
| (20) NEUROD1 | chr2: 181668295-181680665 |
| (21) AC149644.1 | chr2: 238882394-238893337 |
| (22) CCDC48 | chr3: 129001629-129040742 |
| (23) EVX1 | chr7: 27242545-27247819 |
| (24) GHSR | chr3: 172443291-172448456 |
| (25) HSD17B14 | chr19: 48813017-48836677 |
| (26) KRBA1 | chr7: 149715011-149734575 |
| (27) OTOP1 | chr4: 4188726-4226894 |
| (28) PPYR1 | chr10: 47918739-47923524 |
| (29) SRMS | chr20: 63539924-63547504 |
| (30) ZNF582 | chr19: 56382751-56393601 |
| (31) IRX2 | chr5: 2746165-2751655 |
| (32) CSMD1 | chr8: 2935353-4994806 |
| (33) MIR675, H19 | chr11: 1996759-1996831 |
| (34) FOXD3 | chr1: 63323059-63325126 |
| (70) NDRG4 | chr16: 58463645-58513619 |
| (35) NKX2-6 | chr8: 23702451-23706598 |
| (36) PAX1 | chr20: 21705659-21718486 |
| (37) FOXD2 | chr1: 47436017-47440691 |
| (38) SLC6A15 | chr12: 84859488-84912829 |
| (39) PHC2 | chr1: 33323623-33375593 |
| (40) FLRT2 | chr14: 85530025-85628696 |
| (41) GATA2 | chr3: 128479422-128487921 |
| (42) ADCY8 | chr8: 130780301-131040589 |
| (43) CNNM1 | chr10: 99329099-99394330 |
| (44) IKZF1 | chr7: 50304083-50405100 |
| (45) NKX2-3 | chr10: 99532933-99536523 |
| (46) PCDH7 | chr4: 30720415-31146801 |
| (47) SNCB | chr5: 176620084-176630561 |
| (48) ST8SIA1 | chr12: 22193391-22334714 |
| (49) TRAPPC9 | chr8: 139727726-140458579 |
| (50) NKX2-2 | chr20: 21511022-21514026 |
| (51) SLC32A1 | chr20: 38724462-38729372 |
| (52) HOXA5 | chr7: 27141052-27143668 |
| (53) GDNF | chr5: 37812677-37839680 |
| (54) FAT4 | chr4: 125316412-125492932 |
| (55) HOXA2 | chr7: 27100354-27102775 |
| (56) LPHN3 | chr4: 61201258-62072466 |
| (57) ADCYAP1 | chr18: 904943-912172 |
| (58) GRIA2 | chr4: 157220584-157366074 |
| (59) AQP1 | chr7: 30,911,855-30,925,516 |
| (60) BCAT1 | chr12: 24810024-24949459 |
| (61) CYP24A1 | chr20: 54153449-54173977 |
| (62) FOXI2 | chr10: 127737274-127741186 |
| (63) GSX1 | chr13: 27792643-27793952 |
| (64) IRF4 | chr6: 391739-411443 |
| (65) NPY | chr7: 24284188-24291865 |
| (66) PDE1B | chr12: 54561444-54579239 |
| (67) CAHM | chr6: 163413065-163413950 |
| (68) SEPT1N9 | chr17: 77,281,451-77,499,029 |
| (75) sox21 | chr13: 94709625-94712543 |
| (76) slc6a15 | chr12: 84859488-84912829 |
| (77) st8SIAI | chr12: 20063773-22437041 |
| (72) ZSCAN18 | chr19: 58,084,482-58,118,426 |
| (73) COL4AS | chr13: 110,148,958 to 110,307,157 |
| (78) FGF5 | chr4: 80268265-80291017 |
| (74) FOXF1 | chr16: 86510527-86514464 |
| (79) FOXI2 | chr10: 127737274-127741186 |
| (71) SDC2 | chr8: 96493654-96611809 |

Reference to these genes should be understood to include 5 kb upstream of the transcription start site of each of these genes, in particular the promoter region of the gene. Without limiting the present invention to any one theory or mode of action, IKZF1 is generally understood to span chr7:50304782-50405100 (Assembly GRCh38/hg38). This runs from the transcription start site to the polyadenylation site. However, the IKZF1 gene has a further 5′ transcription start site, the coordinates of which, including this start site, are Chr7:50304083-50405101. If the upstream CpG Island is also included, then the coordinates are 50303300-50405101. If the 2 kb upstream sequence is included, then the coordinates are 500302083-50405101.

As will be discussed in more detail hereafter, the method of the present invention can be applied to screening for the methylation of one gene or else it can be adapted to screen a given biological sample for the methylation of more than one gene either via amplification of separate aliquots of DNA from the original biological sample or in the context of a single aliquot which is amplified using a multiplexed amplification method.

Application of amplification methodologies in accordance with the method of the present invention still more unexpectedly achieves an improvement to sensitivity that is significantly greater than the two fold improvement that one might, at best, expect to theoretically achieve by virtue of the amplification of both the target strand and the opposite strand of the non-degraded/nicked DNA region of interest which remains after bisulphite treatment of the starting DNA sample. This result is both unexpected and counter-intuitive when one considers that up to 90% of the starting DNA which is bisulphite treated, prior to amplification, is either degraded or nicked. Accordingly, the present method now enables the routine performance of highly accurate methylation analysis of gene targets which are present in very low starting copy number, such as ccfDNA, for example disease specific ccfDNA, in particular ctDNA. In this regard, reference to “low copy number” should be understood as a reference to a quantity of DNA in a sample which is at or below the level required to achieve a reliable and reproducible amplification results. Without limiting the present invention to any one theory or mode of action, a low level of target DNA in the starting sample is a well understood problem which increases the probability of inaccurate results, in terms of both quantitative and qualitative amplification methods, due to the large number of amplification cycles which are required to generate a detectable level of amplification product. In this regard, determining whether or not a given sample source corresponds to a low copy number sample is routinely determinable by the skilled person. Certain sample types, such as those which are harvested to assess ccfDNA markers, ctDNA markers, minimum residual disease markers, neoplastic clonal evolution markers and the like are well known to exhibit starting levels of the target DNA of interest which are too low to achieve a reliable and accurate result using prior art amplification based methylation analyses. However, there may be other sample sources where the amount of target DNA is unknown. In these situations, the skilled person can apply routine methodology to determine whether the amount of starting target DNA falls within the scope of “low coy number” as defined herein and therefore cannot be reliably analysed by prior art amplification methods. In terms of quantitative PCR, said “low copy number” is also known as the limit of detection (LOD). The LOD is a well understood metric which is determinable by those of skill in the art in relation to an assay or sample of interest. Still without limiting the present invention to anyone theory or mode of action, in a qPCR assay a positive reaction is detected by accumulation of a fluorescent signal. The Ct (cycle threshold) is defined as the number of cycles required for the fluorescent signal to cross the threshold (ie exceeds background level). Ct levels are inversely proportional to the amount of target nucleic acid in the sample (ie the lower the Ct level the greater the amount of target nucleic acid in the sample). Cts <29 are generally regarded as strong positive reactions indicative of abundant target nucleic acid in the sample. Cts of 30-37 are positive reactions usually indicative of moderate amounts of target nucleic acid and Cts of 38-40 are regarded as weak reactions indicative of minimal amounts of target nucleic acid. As the number of Ct cycles required to achieve a result increases, the likely inaccuracy of the result obtained therefrom also increases. As detailed hereinbefore, in term of qPCR (real time PCR), the method of the present invention now enables the accurate and reproducible analysis of target DNA methylation, where that target DNA is present in amounts that fall below the LOD of prior art qPCR methods.

In one embodiment, said low copy number is less than 100 copies of target DNA/sample tested. In another embodiment said low copy number is less than 95 copies of target DNA/sample tested, less than 90 copies of target DNA/sample tested, less than 85 copies of target DNA/sample tested, less than 80 copies of target DNA/sample tested, less than 75 copies of target DNA/sample tested, less than 70 copies of target DNA/sample tested, less than 65 copies of target DNA/sample tested, less than 60 copies of target DNA/sample tested, less than 55 copies of target DNA/sample tested, less than 50 copies of target DNA/sample tested, less than 45 copies of target DNA/sample tested, less than 40 copies of target DNA/sample tested, less than 35 copies of target DNA/sample tested, less than 30 copies of target DNA/sample tested, less than 25 copies of target DNA/sample tested, less than 20 copies of target DNA/sample tested, less than 15 copies of target DNA/sample tested, less than 10 copies of target DNA/sample tested or less than 5 copies of target DNA/sample tested. Most particularly said low copy number is less than 50 copies of target DNA/sample tested, still more particularly less than 40 copies of target DNA/sample tested, yet more particularly less than 30 copies of target DNA/sample tested, still more particularly less than 20 copies of target DNA/sample tested. In a further embodiment, said low copy number is less than 10 copies of target DNA/sample tested, most particularly less than 5 copies of target DNA/sample tested.

In another embodiment, said amplification is quantitative PCR and said low copy number is the LOD.

Accordingly, there is provided a method of screening for the methylation of a gene or region thereof, which gene target is selected from the list consisting essentially of:

| | | | |
|---|---|---|---|
| (1) GRASP | (18) ANGPT2 | (35) NKX2-6 | (52) HOXA5 |
| (2) IRX1 | (19) LHX6 | (36) PAX1 | (53) GDNF |
| (3) SOX21 | (20) NEUROD1 | (37) FOXD2 | (54) FAT4 |
| (4) FGF5 | (21) AC149644.1 | (38) SLC6A15 | (55) HOXA2 |
| (5) ZNF471 | (22) CCDC48 | (39) PHC2 | (56) LPHN3 |
| (6) SUSD5 | (23) EVX1 | (40) FLRT2 | (57) ADCYAP1 |
| (7) FOXB1 | (24) GHSR | (41) GATA2 | (58) GRIA2 |
| (8) PDX1 | (25) HSD17B14 | (42) ADCY8 | (59) AQP1 |
| (9) DLX5 | (26) KRBA1 | (43) CNNM1 | (60) BCAT1 |
| (10) ONECUT2 | (27) OTOP1 | (44) IKZF1 | (61) CYP24A1 |
| (11) DMRTA2 | (28) PPYR1 | (45) NKX2-3 | (62) FOXI2 |
| (12) CMTM2 | (29) SRMS | (46) PCDH7 | (63) GSX1 |
| (13) OTX2 | (30) ZNF582 | (47) SNCB | (64) IRF4 |
| (14) LOC145845 | (31) IRX2 | (48) ST8SIA1 | (65) NPY |
| (15) EBF3 | (32) CSMD1 | (49) TRAPPC9 | (66) PDE1B |
| (16) SALL1 | (33) MIR675, | (50) NKX2-2 | (67) CAHM |
| | H19 | | |
| (17) CBX8 | (34) FOXD3 | (51) SLC32A1 | (68) SEPTIN9 |
| (69) BMP3 | (70) NDRG4 | (71) SDC2 | (72) ZCAN18 |
| (73) COL4A2 | (74) FOXF1 | (75) SOX21 | (76) SLC6A15 |
| (77) ST8SIAI | (78) FGF5 | (79) FOXI2 | |

said method comprising;
(i) contacting a DNA sample with an agent which modifies unmethylated cytosine residues wherein said sample comprises both the target strand and the opposite strand of said gene or region thereof, which DNA sample comprises a low copy number of said gene region of interest;
(ii) contacting the DNA sample of step (i) with:
   a) a first set of forward and reverse primers designed to amplify one or more fully or partially methylated forms of the modified target strand of the gene target;
   b) a second set of forward and reverse primers designed to amplify one or more fully or partially methylated forms of the modified opposite strand of the gene target; and
   c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said gene effects the detection of said hybridised probe; and
(iv) qualitatively or quantitatively analysing the detection output of step (iii).

In another embodiment, said DNA sample is a DNA sample which comprises a low copy number of the DNA region of interest.

In still another embodiment, said DNA region of interest is ccfDNA, such as disease specific ccfDNA, in particular ctDNA.

In another embodiment, said gene is BCAT1, IKZF1, IRF4, GRASP and/or CAHM, in particular BCAT1 and/or IKZF1.

The DNA that is tested in accordance with the method of the present invention may be isolated from a biological sample. Reference to a “biological sample” should be understood as a reference to any sample of biological material derived from any source, such as animal, plant or bacterial, including but not limited to, cellular material, biofluids (e.g. blood, plasma, serum, urine, saliva, ascites fluid, semen), faeces (stool), tissue biopsy specimens, surgical specimens or fluid which has been introduced into the body and subsequently removed (such as, for example, the solution retrieved from an enema wash). The biological sample that is tested according to the method of the present invention may be tested directly or may require some form of treatment prior to testing. For example, a biopsy or surgical sample may require homogenisation prior to testing. Alternatively, a cell sample may require permeabilisation prior to testing. Further, to the extent that the biological sample is not in liquid form, (if such form is required for testing) it may require the addition of a reagent, such as a buffer, to mobilise the sample.

To the extent that the DNA region of interest is present in a biological sample, the biological sample may be directly tested or else all or some of the DNA present in the biological sample may be isolated prior to testing. In yet another example, the sample may be partially purified or otherwise enriched prior to analysis. For example, to the extent that a biological sample comprises a very diverse cell population, it may be desirable to enrich for a sub-population of particular interest. It is within the scope of the present invention for the target biological sample or molecules derived therefrom to be treated prior to testing, for example, inactivation of live virus. It should also be understood that the biological sample may be freshly harvested or it may have been stored (for example by freezing) prior to testing or otherwise treated prior to testing (such as by undergoing culturing).

The choice of what type of sample is most suitable for testing in accordance with the method disclosed herein will be dependent on the nature of the situation. To the extent that one is screening for the onset or predisposition to the onset of a large intestine neoplasm, for example, said sample is preferably a faecal (stool) sample, enema wash, surgical resection, tissue biopsy or biofluid such as urine, saliva, ascites fluid or blood sample (e.g. whole blood, serum or plasma).

More preferably, said biological sample is a blood sample, plasma, serum, saliva, stool, ascites fluid, urine, biopsy sample, or stool sample.

The sample of the present invention comprises both the target strand and the opposite strand of the DNA region of interest. As would be understood by the person of skill in the art, chromosomal DNA comprises two complimentary strands of DNA which hybridise together to form a molecule. The DNA region which is the subject of interest is defined, in the context of the present invention, as the “target strand” while the complementary strand is referred to as the “opposite strand”. The skilled person would appreciate that the two strands of a DNA double helix are also often referred to as the “sense” strand, “coding” strand, “positive (+)” strand, “top” strand, or “upper” strand. These latter three terms are more commonly utilised where the DNA region of interest does not produce a protein expression product. The corresponding complementary strand is often referred to as the “antisense” strand, “non-coding” strand, “negative (−)” strand, “lower” strand or “bottom” strand. This should be understood to mean the strand which, in the context of the chromosomal locus, is complementary to the top/+/upper strand and, in its natural state, hybridises to the top strand to form the characteristic double helix structure. As would be appreciated by the person of skill in the art, this nomenclature has become progressively less precise as it has been determined that there are many gene regions that do not code for proteins (and are not therefore correctly described as being found on the sense or coding strand) and, further, that genes may be found on either the +/upper strand or the −/lower strand, depending on how the skilled person defines these strands. Even genes which code for proteins are now known to be found on what was traditionally regarded as the −/bottom/antisense strand. Accordingly, identifying and defining a strand by reference to this terminology alone, and without reference to a specific chromosomal position or by reference to the specific +/− strand nomenclature used in the annotated human genome data base, may be imprecise. In this regard, in the context of the present invention a reference to the “target strand” is a reference to the DNA strand which comprises the region of interest, whichever of the two strands this is, while the “opposite strand” is a reference to the complementary strand. The target strand may therefore correspond to either the +/− (top/bottom, upper/lower) strand, depending on where the gene is positioned on the chromosomal double helix.

It should be understood that the target and opposite strands of the biological sample of step (i) of the method of the present invention may be in either hybridised double stranded form or non-hybridised single stranded form prior to contact with an agent which modifies the unmethylated cytosine residues of said DNA. Whether or not the subject DNA is in double stranded or single stranded form is likely to be dependent on what, if any, treatment the sample underwent prior to the commencement of testing in accordance with the method if the present invention. Still further, depending on the agent which is selected to effect the unmethylated cytosine modification, the skilled person may elect to manipulate the DNA of the sample in order to facilitate the formation of a double stranded or single stranded form.

As detailed hereinbefore, the method of the present invention provides a means of accurately qualitatively or quantitatively analysing the methylation characteristics of a DNA target via amplification-based methodology. By applying the method of the present, the results are now significantly less skewed as a result of sugar phosphate DNA backbone nicking during the bisulphite treatment process. In terms of applying this method it should be appreciated by the person of skill in the art that any of the existing amplification methods which are designed to interrogate the methylation of a double stranded DNA sequence, via a combination of amplification and probing, can be adapted in accordance with the method of the present invention. For example, one can design an amplification method (such as PCR) that uses either methylation specific primers or non-methylation specific primers. In accordance with the exemplified embodiment, methylation specific primers are used (e.g. methylation-specific PCR). However, non-methylation specific primers could also be used, although in this case the methylation interrogation will rely solely on the results obtained from the use of methylation-specific probes since these primers will amplify the target DNA regardless of whether or not it is methylated. Similarly, in terms of the probes that are used, the exemplified embodiment uses hydrolysis probes, which enable real-time PCR quantification to be achieved. However, even where such probes are used, it may be sufficient to qualitatively analyse the readout that is obtained. Alternatively, one may elect to use a probe that only provides a qualitative readout and does not enable quantitative analysis.

In a first step, the nucleic acid sample that is the subject of analysis is contacted with an agent to modify unmethylated cytosine residues. The term “modifies” as used herein means the conversion of an unmethylated cytosine to another nucleotide by an agent, said conversion distinguishing unmethylated from methylated cytosine in the original nucleic acid sample. Any suitable agent may be used. In one embodiment, the agent is one that converts unmethylated cytosine to uracil, such as sodium bisulphite. However, other equivalent modifying agents that selectively modify unmethylated cytosine, but not methylated cytosine, can be used in the method of the invention. For example, one can use any other suitable form of bisulphite, such as ammonium bisulphite. Sodium-bisulphite readily reacts with the 5, 6-double bond of cytosine, but not with methylated cytosine, to produce a sulfonated cytosine intermediate that undergoes deamination under alkaline or high temperature conditions to produce uracil. Because Taq polymerase recognises uracil as thymine and 5-methylcytosine (m5C) as cytosine, the sequential combination of sodium bisulphite treatment and PCR amplification results in the ultimate conversion of unmethylated cytosine residues to thymine (C→U→T) and methylated cytosine residues (“mC”) to cytosine (mC→mC→C). Thus, sodium-bisulphite treatment of genomic DNA creates methylation-dependent sequence differences by converting unmethylated cytosines to uracil. It should be understood that in terms of the hybridising of primers to the nucleic acid of step (i), the primers are designed to hybridise to the modified (eg. bisulphite-converted) DNA, or the DNA amplified therefrom. In this regard, the primers may be designed to function as methylation specific primers or non-methylation specific primers depending on the design of the method.

According to this embodiment there is provided a method of screening for the methylation of a DNA region of interest, said method comprising:
(i) contacting a DNA sample with a bisulphite agent to convert unmethylated cytosine residues to uracil wherein said sample comprises both the target strand and the opposite strand of said DNA region of interest, which DNA sample comprises a low copy number of said DNA region of interest;
(ii) contacting the DNA sample of step (i) with:
   a) a first set of forward and reverse primers designed to amplify one or more fully or partially methylated forms of the modified target strand of the DNA region of interest;
   b) a second set of forward and reverse primers designed to amplify one or more fully or partially methylated forms of the modified opposite strand of the DNA region of interest; and
   c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said gene effects the detection of said hybridised probe; and
(iv) qualitatively or quantitatively analysing the detection output of step (iii).

In one embodiment, said DNA region of interest is a gene target.

In another embodiment, said gene target is one or more of:

| | | | |
|---|---|---|---|
| (1) GRASP | (18) ANGPT2 | (35) NKX2-6 | (52) HOXA5 |
| (2) IRX1 | (19) LHX6 | (36) PAX1 | (53) GDNF |
| (3) SOX21 | (20) NEUROD1 | (37) FOXD2 | (54) FAT4 |
| (4) FGF5 | (21) AC149644.1 | (38) SLC6A15 | (55) HOXA2 |
| (5) ZNF471 | (22) CCDC48 | (39) PHC2 | (56) LPHN3 |
| (6) SUSD5 | (23) EVX1 | (40) FLRT2 | (57) ADCYAP1 |
| (7) FOXB1 | (24) GHSR | (41) GATA2 | (58) GRIA2 |
| (8) PDX1 | (25) HSD17B14 | (42) ADCY8 | (59) AQP1 |
| (9) DLX5 | (26) KRBA1 | (43) CNNM1 | (60) BCAT1 |
| (10) ONECUT2 | (27) OTOP1 | (44) IKZF1 | (61) CYP24A1 |
| (11) DMRTA2 | (28) PPYR1 | (45) NKX2-3 | (62) FOXI2 |
| (12) CMTM2 | (29) SRMS | (46) PCDH7 | (63) GSX1 |
| (13) OTX2 | (30) ZNF582 | (47) SNCB | (64) IRF4 |
| (14) LOC145845 | (31) IRX2 | (48) ST8SIA1 | (65) NPY |
| (15) EBF3 | (32) CSMD1 | (49) TRAPPC9 | (66) PDE1B |
| (16) SALL1 | (33) MIR675, | (50) NKX2-2 | (67) CAHM |
| | H19 | | |
| (17) CBX8 | (34) FOXD3 | (51) SLC32A1 | (68) SEPTIN9 |
| (69) BMP3 | (70) NDRG4 | (71) SDC2 | (72) ZCAN18 |
| (73) COL4A2 | (74) FOXF1 | (75) SOX21 | (76) SLC6A15 |
| (77) ST8SIAI | (78) FGF5 | (79) FOXI2 | |

In still another embodiment, said gene target is one or more of BCAT1, IKZF1, IRF4, GRASP or CAHM, in particular BCAT1 and/or IKZF1.

In yet another embodiment, the result of step (iv) exhibits a reduced incidence of false negative results.

In yet another embodiment, said DNA sample is blood, plasma, serum, saliva, stool, ascites fluid or urine.

In still further embodiment, said DNA region of interest is ccfDNA, such as disease specific ccfDNA, in particular ctDNA.

In one embodiment, said low copy number is less than 100 copies of target DNA/sample tested. In another embodiment said low copy number is less than 95 copies of target DNA/sample tested, less than 90 copies of target DNA/sample tested, less than 85 copies of target DNA/sample tested, less than 80 copies of target DNA/sample tested, less than 75 copies of target DNA/sample tested, less than 70 copies of target DNA/sample tested, less than 65 copies of target DNA/sample tested, less than 60 copies of target DNA/sample tested, less than 55 copies of target DNA/sample tested, less than 50 copies of target DNA/sample tested, less than 45 copies of target DNA/sample tested, less than 40 copies of target DNA/sample tested, less than 35 copies of target DNA/sample tested, less than 30 copies of target DNA/sample tested, less than 25 copies of target DNA/sample tested, less than 20 copies of target DNA/sample tested, less than 15 copies of target DNA/sample tested, less than 10 copies of target DNA/sample tested or less than 5 copies of target DNA/sample tested. Most particularly said low copy number is less than 50 copies of target DNA/sample tested, still more particularly less than 40 copies of target DNA/sample tested, yet more particularly less than 30 copies of target DNA/sample tested, still more particularly less than 20 copies of target DNA/sample tested. In a further embodiment, said low copy number is less than 10 copies of target DNA/sample tested, most particularly less than 5 copies of target DNA/sample tested.

In another embodiment, said amplification is quantitative PCR and said low copy number is the LOD. Once the conversion of the unmethylated cytosine residues has been effected, the sample is ready for amplification. As detailed hereinbefore, the present invention is predicated on the determination that the output of amplification-based methylation analysis can be unexpectedly significantly increased, and the incidence of false negative results significantly reduced, where the amplification reaction is designed to use at least one set of primers and probes which are designed to hybridise to the modified target strand and a further set of primers and probes which are designed to hybridise to the modified opposite strand of the DNA region of interest, these strands no longer being complementary after bisulphite treatment, thereby enabling the methylation analysis of both the target and the opposite strands to proceed independently. In the context of clinical diagnostic applications, directed to detecting low copy number gene targets as markers of disease, the method of the present invention enables not only the generation of amplification product, and therefore results, where previously this may have been prevented due to generalised DNA degradation events reducing the levels of already very low levels of starting DNA target material, but even more importantly producing results which are significantly less affected by the actions of random nicking of the target strand, which can lead to the generation of false negative results, such as where the nick occurs within a primer or probe hybridisation site..

In this regard, reference to a “primer” should be understood as a reference to any molecule comprising a sequence of nucleotides, or functional derivatives or analogues thereof, the function of which includes both annealing to a complementary DNA sequence which flanks the methylation region of interest and amplification of the DNA sequence downstream of the annealing region. It should be understood that the primer may comprise non-nucleic acid components. For example, the primer may also comprise a non-nucleic acid tag such as a fluorescent or enzymatic tag or some other non-nucleic acid component that facilitates the use or detection of the molecule. In another example, the primer may be a protein nucleic acid that comprises a peptide backbone exhibiting nucleic acid side chains. Preferably, said primer is a single stranded DNA oligonucleotide.

The design and synthesis of primers suitable for use in the present invention would be well known to those of skill in the art. In one embodiment, the subject primer is 4 to 60 nucleotides in length, in another embodiment 10 to 50 nucleotides in length, in yet another embodiment 15 to 45 nucleotides in length, and in still another embodiment 20 to 40 nucleotides in length. In terms of the number of primers that are used in the method of the invention, this can be determined by the person of skill in the art. With regard to the total number of primers, the variables that require consideration are the size and number of nucleic acid regions that are being amplified and the distance between the sequences to which the primers hybridise. To the extent that a nested PCR reaction is performed, it should be understood that reference to a “set” of primers directed to either the target strand or the opposite strand is a reference to all of the primers that are to be used for a given nested reaction to one of these strands and not just the outermost forward and reverse primers. It should also be understood that irrespective of how many primers may be selected for use as part of a set to amplify a given DNA strand, for example in the context of the design of nested reactions, the sequences of at least some of the first set will differ to the sequence of at least some of the primers of the second set in that the former are designed to selectively amplify the target strand and the latter are designed to selectively amplify the opposite strand. It is not inconceivable, however, that if internal nested primers are elected to be used, that these may be the same for both strands, depending on the nature of the sequences of the template strands.

In one embodiment, the oligonucleotide primers are linear, single-stranded oligomeric deoxyribonucleic or ribonucleic acid molecules capable of sequence-specific hybridisation with complementary strands of nucleic acid. The primers are preferably DNA. The primers of the invention are of sufficient length to provide for specific and efficient initiation of polymerization (primer extension) during the amplification process. The exact length will depend on multiple factors including temperature (during amplification), buffer, and nucleotide composition. Preferably, the primers are single-stranded although double-stranded primers may be used if the strands are first separated.

Primers may be prepared using any suitable method, such as conventional phosphotriester and phosphodiester methods or automated methods, which are commonly known in the art.

As detailed hereinbefore, the primers that are utilised in the method of the present invention may be any suitable primers that amplify the nucleic acid target of interest. For example, the primers may be methylation-specific primers or non-methylation specific primers. By “methylation-specific” primers is meant primers which can distinguish between methylated and non-methylated DNA, such as bisulphite converted methylated vs non-methylated DNA. Such methylation specific primers can be designed to distinguish between methylated and non-methylated DNA by, for example, hybridising with only unconverted 5-methylcytosines (i.e. the primer hybridises to bisulphite-converted methylated DNA) or, conversely, hybridising to thymines that are converted from unmethylated cytosines (i.e. the primer hybridises to bisulphite-converted unmethylated DNA). Methylation is thereby determined by the ability of the specific primer to achieve amplification. As would be appreciated by the person of skill in the art, in order to achieve methylation-specific discrimination the primers are preferably designed to overlap potential sites of DNA methylation (CpG dinucleotides) and to specifically distinguish modified unmethylated from methylated DNA. For example, the primers may be designed to overlap one to several CpG sequences, preferably one to five CpG sequences or one to four CpG sequences.

It would be appreciated that where non-methylation specific primers are used, it is necessary that the panel of probes that is utilised does not include a probe that is unable to discriminate between methylated and unmethylated DNA. In this regard, it should be understood that where non-methylation specific primers are used, the first set of primers and the second set of primers may be the same since the fact of targeting a non-methylation specific region of a bisulphite converted target strand will mean that the corresponding region on the opposite strand will still be complementary after bisulphite conversion. It is well within the skill of the person in the art to design a probe set in accordance with the present invention and which detects two or more methylation patterns for a nucleic acid region of interest but which does not detect unmethylated DNA. Where the primers that are used are methylation specific, the issue of whether or not the probe set includes a probe directed to the non-methylated form of the nucleic acid target of interest is less significant.

Reference to the primers being “designed to amplify one or more fully or partially methylated forms” of the DNA region of interest should be understood to mean that the primers will enable amplification of either all or just some of the methylated forms of the subject region, these amplicons being thereafter interrogated by a probe. If the primer is non-methylation specific, it will amplify all of the forms of the subject region, irrespective of the existence or not of any degree of methylation. For example, the primers may be designed such that they hybridise to unmethylated DNA regions which are located upstream and downstream to the CpGs which form part of the region of partial cytosine methylation. In this situation, the primers will amplify this region of all the nucleic acid molecules present in the sample since the primers have been designed to hybridise to a DNA site which is unmethylated but which is located proximally to the methylated region of cytosines. In this case, the methylation specificity of the method will be provided only by the probes and it would be important to ensure that the pool of probes does not include a probe directed to a fully unmethylated form of the target region. In another embodiment one or more of the primers may be methylation specific and designed to hybridise to one or more of the cytosine residues which are fully methylated and which lie upstream and/or downstream of the region of partial methylation. By designing methylation-specific primers, methylation specific amplification can be achieved. In yet another example, one or both of the primers may be directed to the partially methylated residues themselves. In this situation, in order to achieve good sensitivity it is desirable to design a primer which hybridises promiscuously, or a pool of primers, which will hybridise to, and enable amplification of, as many different partially methylated forms of the DNA target as possible, thereby improving specificity. This may be achieved, for example, in the context of the application of a multiplexed assay. In terms of the design of either a suitable promiscuous primer or pool of primers, the description provided hereafter in relation to probe sequence design is also applicable to the design of these primers, both molecules being oligonucleotides which are designed to hybridise to a target DNA region. The design of such primers and probes is discussed in detail in Patent Publication No. WO 2015/184498.

In one embodiment, said primers are methylation specific.

According to this embodiment there is provided a method of screening for the methylation of a DNA region of interest, said method comprising:
(i) contacting a DNA sample with a bisulphite agent to convert unmethylated cytosine residues to uracil wherein said sample comprises both the target strand and the opposite strand of said DNA region of interest, which DNA sample comprises a low copy number of said DNA region of interest;
(ii) contacting the DNA sample of step (i) with:
   a) a first set of methylation specific forward and reverse primers designed to amplify one or more fully or partially methylated forms of the modified target strand of the DNA region of interest;
   b) a second set of methylation specific forward and reverse primers designed to amplify one or more fully or partially methylated forms of the modified opposite strand of the DNA region of interest; and
   c) optionally one or more probes which incorporate a detection means;
(iii) amplifying the DNA sample of step (ii) wherein if one or more of the probes of step (ii)(c) are used, the extension of said primers along said DNA effects the detection of said hybridised probe; and
(iv) qualitatively or quantitatively analysing the detection output of step (iii).

In another embodiment, said agent that modifies unmethylated cytosine residues is sodium bisulphite.

In yet another embodiment, said DNA region of interest is a gene target or region thereof.

In still another embodiment, said gene target is selected from the list consisting essentially of:

| | | | |
|---|---|---|---|
| (1) GRASP | (18) ANGPT2 | (35) NKX2-6 | (52) HOXA5 |
| (2) IRX1 | (19) LHX6 | (36) PAX1 | (53) GDNF |
| (3) SOX21 | (20) NEUROD1 | (37) FOXD2 | (54) FAT4 |
| (4) FGF5 | (21) AC149644.1 | (38) SLC6A15 | (55) HOXA2 |
| (5) ZNF471 | (22) CCDC48 | (39) PHC2 | (56) LPHN3 |
| (6) SUSD5 | (23) EVX1 | (40) FLRT2 | (57) ADCYAP1 |
| (7) FOXB1 | (24) GHSR | (41) GATA2 | (58) GRIA2 |
| (8) PDX1 | (25) HSD17B14 | (42) ADCY8 | (59) AQP1 |
| (9) DLX5 | (26) KRBA1 | (43) CNNM1 | (60) BCAT1 |
| (10) ONECUT2 | (27) OTOP1 | (44) IKZF1 | (61) CYP24A1 |
| (11) DMRTA2 | (28) PPYR1 | (45) NKX2-3 | (62) FOXI2 |
| (12) CMTM2 | (29) SRMS | (46) PCDH7 | (63) GSX1 |
| (13) OTX2 | (30) ZNF582 | (47) SNCB | (64) IRF4 |
| (14) LOC145845 | (31) IRX2 | (48) ST8SIA1 | (65) NPY |
| (15) EBF3 | (32) CSMD1 | (49) TRAPPC9 | (66) PDE1B |
| (16) SALL1 | (33) MIR675, | (50) NKX2-2 | (67) CAHM |
| | H19 | | |
| (17) CBX8 | (34) FOXD3 | (51) SLC32A1 | (68) SEPT1N9 |
| (69) BMP3 | (70) NDRG4 | (71) SDC2 | (72) ZCAN18 |
| (73) COL4A2 | (74) FOXF1 | (75) SOX21 | (76) SLC6A15 |
| (77) ST8SIAI | (78) FGF5 | (79) FOXI2 | |

In yet still another embodiment, said gene is one or more of BCAT1, IKZF1, IRF4, GRASP or CAHM, in particular BCAT1 and/or IKZF1.

In yet another embodiment, the result of step (iv) exhibits a reduced incidence of false negative results.

In still another embodiment, said DNA sample is blood, plasma, serum, saliva, stool, ascites fluid or urine.

In still further embodiment, said DNA region of interest is ccfDNA, such as disease specific ccfDNA, in particular ctDNA.

In yet a further embodiment, said probe is a non-methylation specific probe.

In one embodiment, said low copy number is less than 100 copies of target DNA/sample tested. In another embodiment said low copy number is less than 95 copies of target DNA/sample tested, less than 90 copies of target DNA/sample tested, less than 85 copies of target DNA/sample tested, less than 80 copies of target DNA/sample tested, less than 75 copies of target DNA/sample tested, less than 70 copies of target DNA/sample tested, less than 65 copies of target DNA/sample tested, less than 60 copies of target DNA/sample tested, less than 55 copies of target DNA/sample tested, less than 50 copies of target DNA/sample tested, less than 45 copies of target DNA/sample tested, less than 40 copies of target DNA/sample tested, less than 35 copies of target DNA/sample tested, less than 30 copies of target DNA/sample tested, less than 25 copies of target DNA/sample tested, less than 20 copies of target DNA/sample tested, less than 15 copies of target DNA/sample tested, less than 10 copies of target DNA/sample tested or less than 5 copies of target DNA/sample tested. Most particularly said low copy number is less than 50 copies of target DNA/sample tested, still more particularly less than 40 copies of target DNA/sample tested, yet more particularly less than 30 copies of target DNA/sample tested, still more particularly less than 20 copies of target DNA/sample tested. In a further embodiment, said low copy number is less than 10 copies of target DNA/sample tested, most particularly less than 5 copies of target DNA/sample tested.

In another embodiment, said amplification is quantitative PCR and said low copy number is the LOD.

In yet another embodiment there is provided a method of screening for the methylation of a DNA region of interest, said method comprising:
(i) contacting a DNA sample with a bisulphite agent to convert unmethylated cytosine residues to uracil wherein said sample comprises both the target strand and the opposite strand of said DNA region of interest, which DNA sample comprises a low copy number of said DNA region of interest;
(ii) contacting the DNA sample of step (i) with:
   a) a first set of non-methylation specific forward and reverse primers designed to amplify one or more fully or partially methylated forms of the modified target strand of the DNA region of interest;
   b) a second set of non-methylation specific forward and reverse primers designed to amplify one or more fully or partially methylated form of the modified opposite strand of the DNA region of interest; and
   c) one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said DNA effects the detection of said hybridised probe; and
(iv) qualitatively or quantitatively analysing the detection output of step (iii).

In still another embodiment, said agent that modifies unmethylated cytosine residues is sodium bisulphite.

In one embodiment, said DNA region of interest is a gene target.

In another embodiment, said gene target is selected from the list consisting essentially of:

| | | | |
|---|---|---|---|
| (1) GRASP | (18) ANGPT2 | (35) NKX2-6 | (52) HOXA5 |
| (2) IRX1 | (19) LHX6 | (36) PAX1 | (53) GDNF |
| (3) SOX21 | (20) NEUROD1 | (37) FOXD2 | (54) FAT4 |
| (4) FGF5 | (21) AC149644.1 | (38) SLC6A15 | (55) HOXA2 |
| (5) ZNF471 | (22) CCDC48 | (39) PHC2 | (56) LPHN3 |
| (6) SUSD5 | (23) EVX1 | (40) FLRT2 | (57) ADCYAP1 |
| (7) FOXB1 | (24) GHSR | (41) GATA2 | (58) GRIA2 |
| (8) PDX1 | (25) HSD17B14 | (42) ADCY8 | (59) AQP1 |
| (9) DLX5 | (26) KRBA1 | (43) CNNM1 | (60) BCAT1 |
| (10) ONECUT2 | (27) OTOP1 | (44) IKZF1 | (61) CYP24A1 |
| (11) DMRTA2 | (28) PPYR1 | (45) NKX2-3 | (62) FOXI2 |
| (12) CMTM2 | (29) SRMS | (46) PCDH7 | (63) GSX1 |
| (13) OTX2 | (30) ZNF582 | (47) SNCB | (64) IRF4 |
| (14) LOC145845 | (31) IRX2 | (48) ST8SIA1 | (65) NPY |
| (15) EBF3 | (32) CSMD1 | (49) TRAPPC9 | (66) PDE1B |
| (16) SALL1 | (33) MIR675, | (50) NKX2-2 | (67) CAHM |
| | H19 | | |
| (17) CBX8 | (34) FOXD3 | (51) SLC32A1 | (68) SEPTIN9 |
| (69) BMP3 | (70) NDRG4 | (71) SDC2 | (72) ZCAN18 |
| (73) COL4A2 | (74) FOXF1 | (75) SOX21 | (76) SLC6A15 |
| (77) ST8SIAI | (78) FGF5 | (79) FOXI2 | |

In yet still another embodiment, said gene is one or more of BCAT1, IKZF1, IRF4, GRASP or CAHM, in particular BCAT1 and/or IKZF1.

In still another embodiment, said DNA sample is blood, plasma, serum, saliva, stool, ascites fluid or urine.

In yet another embodiment, the result of step (iv) exhibits a reduced incidence of false negative results.

In still further embodiment, said DNA region of interest is ccfDNA, such as disease specific ccfDNA, in particular ctDNA.

In one embodiment, said low copy number is less than 100 copies of target DNA/sample tested. In another embodiment said low copy number is less than 95 copies of target DNA/sample tested, less than 90 copies of target DNA/sample tested, less than 85 copies of target DNA/sample tested, less than 80 copies of target DNA/sample tested, less than 75 copies of target DNA/sample tested, less than 70 copies of target DNA/sample tested, less than 65 copies of target DNA/sample tested, less than 60 copies of target DNA/sample tested, less than 55 copies of target DNA/sample tested, less than 50 copies of target DNA/sample tested, less than 45 copies of target DNA/sample tested, less than 40 copies of target DNA/sample tested, less than 35 copies of target DNA/sample tested, less than 30 copies of target DNA/sample tested, less than 25 copies of target DNA/sample tested, less than 20 copies of target DNA/sample tested, less than 15 copies of target DNA/sample tested, less than 10 copies of target DNA/sample tested or less than 5 copies of target DNA/sample tested. Most particularly said low copy number is less than 50 copies of target DNA/sample tested, still more particularly less than 40 copies of target DNA/sample tested, yet more particularly less than 30 copies of target DNA/sample tested, still more particularly less than 20 copies of target DNA/sample tested. In a further embodiment, said low copy number is less than 10 copies of target DNA/sample tested, most particularly less than 5 copies of target DNA/sample tested.

In another embodiment, said amplification is quantitative PCR and said low copy number is the LOD.

The size of the DNA regions to be amplified by the method of the present invention can be determined by the person of skill in the art and will depend upon factors such as the size of the region to which the probe must bind and the distribution, along the DNA target sequence, of the CpG dinucleotide clusters to which the primers are directed. To this end the amplification method of the present invention is designed such that the probe is directed to a DNA sequence region between the primers (i.e. an inter-primer sequence) or a region which overlaps with a primer region and will therefore selectively hybridise to the amplicons that are produced as a result of amplification.

It should be understood that reference to the forward and reverse primers being “directed to” the target of interest should be understood to mean that the primers hybridise and amplify either all or part of the target in issue. For example, where the target of interest is a gene target, the primers may be designed to hybridise to and amplify a smaller section subregion of the gene, such as all or part of the promoter region. As would be appreciated by the person of skill in the art, it is generally desirable to generate and analyse smaller sized amplicons rather than large amplicons.

As detailed hereinbefore, the method of the present invention provides a reliable and significantly more sensitive and accurate means of quantitatively or qualitatively screening for a methylated DNA target, in particular one which is present in low initial copy number. To the extent that quantitative analysis is sought to be performed, the methylation analysis assay is required to incorporate a detectable probe designed to hybridise to the amplicons of interest which are generated by the amplification step. Accordingly, reference to “probes” should be understood as a reference to any molecule comprising a sequence of nucleotides, or functional derivatives or analogues thereof, the function of which includes the hybridisation of at least one region of said nucleotide sequence with a target nucleic acid molecule. As detailed hereinbefore, the method of the present invention provides a reliable and accurate means of quantitatively (or qualitatively) screening for a methylated DNA target even where partial methylation exists. This is enabled by virtue of the design and application of a probe or pool of probes that are designed to detect all potential partial methylation patterns for a given region of interest. It has been further determined that the use of a heterogeneous pool of probes of this type does hybridise effectively to, and enable detection of, the entire range of partially methylated forms of DNA which are present in the DNA sample being screened.

Accordingly, in another embodiment said probes are one or more hydrolysis probes directed to a region of partial cytosine methylation wherein said one or more probes collectively hybridise to at least two differing methylation patterns at said region.

The nucleic acid probe may comprise non-nucleic acid components. Specifically, the nucleic acid probe also comprises a detection means, such as a fluorescent tag or some other component that facilitates the functioning of the molecule, such as the detection or immobilisation of the molecule. Reference to “detection means” should be understood as a reference to the incorporation of any means that enables detection of the probe. The detection means may facilitate either qualitative or quantitative detection, although quantitative is of particular utility. The detection means may take the form of a detectable moiety or agent, such as a fluorophore or radioisotope. Alternatively, the detection means may enable the physical isolation of the probe, from the reaction mixture, for analysis, such as via magnetic beads or a biotin-streptavidin system.

Without limiting the present invention in any way, the individual probe components can be either all labelled with the same detection agent (e.g. fluorophore) or each probe component can be labelled with a different agent (e.g. different emission wavelength fluorophores). For example, a probe mixture may be designed such that all probe components are labelled with the same fluorophore, even if there is more than one specificity of probe present in the mixture, and thus any one or more of the probes that binds will give a positive signal in real-time PCR. An alternative approach is to attach different fluorophores to different probes and to discriminate between the probe specificities which hybridise. For example, a heterogeneous probe mixture designed to identify multiple different partially methylated forms of the gene of interest, discriminate between bases that are methylated (or not) based on the wavelength(s) detected. This approach may be informative for cancer staging if, for instance, partial methylation was a feature of early-stage cancers and full methylation a feature of later stage cancers. Current real-time PCR instruments can detect up to six different fluorophores, but other techniques are available to interrogate multiple features in one sample (bead-based fluorescent sorting, for example). In such a case, each probe could be attached to a bead that could be sorted independently.

For example, the present invention encompasses the use of real-time quantitative forms of PCR, such as, for example, TaqMan (Holland et al., Proc. Natl. Acad. Sci. USA, 88, 7276-7280, 1991; Lee et al., Nucleic Acid Res. 21, 3761-3766, 1993) to perform this embodiment. For example, the MethyLight method of Eads et al., Nucl. Acids Res. 28: E32, 2000 uses a modified TaqMan hydrolysis-probe assay to detect methylation of a CpG dinucleotide. Essentially, this method comprises treating a nucleic acid sample with bisulphite and amplifying nucleic acid comprising one or more CpG dinucleotides that are methylated in a neoplastic cell and not in a control sample using an amplification reaction, e.g., PCR. The amplification reaction is performed in the presence of three oligonucleotides, a forward and reverse primer that flank the region of interest and a probe that hybridizes between the two primers to the site of the one or more methylated CpG dinucleotides. The probe is dual labelled with a 5′ fluorescent reporter and a 3′ quencher (or vice versa). When the probe is intact, the quencher dye absorbs the fluorescence of the reporter due to their proximity. Following annealing of to the PCR product the probe is cleaved by 5′ to 3′ exonuclease activity of, for example, Taq DNA polymerase. This cleavage releases the reporter from the quencher thereby resulting in an increased fluorescence signal that can be used to estimate the initial template methylation level. By using a probe or primer that selectively hybridizes to unmutated nucleic acid (i.e. methylated nucleic acid) the level of methylation is determined, e.g., using a standard curve.

Alternatively, rather than using a labelled probe that requires cleavage, a probe, such as, for example, a Molecular Beacon is used (see, for example, Mhlanga and Malmberg, Methods 25:463-471, 2001). Molecular beacons are single stranded nucleic acid molecules with a stem-and-loop structure. The loop structure is complementary to the region surrounding the one or more CpG dinucleotides that are methylated in a neoplastic sample and not in a control sample. The stem structure is formed by annealing two “arms” complementary to each other, which are on either side of the probe (loop). A fluorescent moiety is bound to one arm and a quenching moiety that suppresses any detectable fluorescence when the molecular beacon is not bound to a target sequence is bound to the other arm. Upon binding of the loop region to its target nucleic acid the arms are separated and fluorescence is detectable. However, even a single base mismatch significantly alters the level of fluorescence detected in a sample. Accordingly, the presence or absence of a particular base is determined by the level of fluorescence detected. Such an assay facilitates detection of one or more unmutated sites (i.e. methylated nucleotides) in a nucleic acid.

Fluorescently labelled locked nucleic acid (LNA) molecules or fluorescently labelled protein-nucleic acid (PNA) molecules are useful for the detection of nucleotide differences (e.g., as described in Simeonov and Nikiforov, Nucleic Acids Research, 30(17): 1-5, 2002). LNA and PNA molecules bind, with high affinity, to nucleic acid, in particular, DNA. Fluorophores (in particular, rhodomine or hexachlorofluorescein) conjugated to the LNA or PNA probe fluoresce at a significantly greater level upon hybridization of the probe to target nucleic acid. However, the level of increase of fluorescence is not enhanced to the same level when even a single nucleotide mismatch occurs. Accordingly, the degree of fluorescence detected in a sample is indicative of the presence of a mismatch between the LNA or PNA probe and the target nucleic acid, such as, in the presence of a methylated cytosine in a CpG dinucleotide. Preferably, fluorescently labelled LNA or PNA technology is used to detect at least a single base change in a nucleic acid that has been previously amplified using, for example, an amplification method known in the art and/or described herein.

As will be apparent to the skilled artisan, LNA or PNA detection technology is amenable to a high-throughput detection of one or more markers by immobilizing an LNA or PNA probe to a solid support, as described in Orum et al., Clin. Chem. 45: 1898-1905, 1999.

Preferably, methylation-dependent sequence differences are detected by methods based on fluorescence-based quantitative PCR (real-time quantitative PCR, Heid et al., Genome Res. 6:986-994, 1996; Gibson et al., Genome Res. 6:995-1001, 1996) (e.g., “TaqMan®”, and “Lightcycler®” technologies). For the TaqMan® and Lightcycler® technologies, the sequence discrimination can occur at either or both of two steps: (1) the amplification step, or (2) the fluorescence detection step. In the case of the FRET hybridisation, probes format on the Lightcycler®, either or both of the FRET oligonucleotides can be used to distinguish the sequence difference. Most preferably the amplification process, as employed in all inventive embodiments herein, is that of fluorescence-based Real Time Quantitative PCR (Heid et al., Genome Res. 6:986-994, 1996) and employ a dual-labelled fluorescent oligonucleotide probe (TaqMan® PCR, using an ABI Prism 7700 Sequence Detection System, Perkin Elmer Applied Biosystems, Foster City, Calif.).

In one embodiment, the detection means is a fluorescent reporter molecule, more preferably, a hydrolysis probe. Reference to “hydrolysis probe” should be understood as a reference to a dual-labelled TaqMan® oligonucleotide. Without limiting the present invention to any one theory or mode of action, the 5′ end of the oligonucleotide is labelled with a fluorescent reporter molecule while the 3′ end is labelled with a quencher molecule. The sequence of the probe is specific for the region of interest in the amplified target molecule. The hydrolysis probe is designed so that the length of the sequence places the 5′ fluorophore and the 3′ quencher in close enough proximity so as to suppress fluorescence.

Hydrolysis probes are designed to bind a region of interest between the binding sites for the PCR amplification primers. During the extension phase of the PCR cycle Taq DNA polymerase synthesises the complementary strand downstream of the PCR primers. When the extension reaches the bound hydrolysis probe the 5′-3′ exonuclease activity of the Taq DNA polymerase degrades the hydrolysis probe. Cleavage of the probe separates the fluorescent reporter molecule from the rest of the probe (and therefore the quencher) allowing the reporter molecule to fluoresce. The Taq DNA polymerase continues synthesising the rest of the nascent strand, thus hybridisation of the probe does not inhibit the PCR reaction. With subsequent PCR cycles the amount of fluorescent report released, and hence fluorescence, increases cumulatively. Examples of suitable reporter and quencher molecule are: the 5′ fluorescent reporter dyes 6FAM (“FAM”; 2,7 dimethoxy-4,5-dichloro-5-carboxy-fluorescein), HEX, Texas Red, TEX615, Cy5 and TET (6-carboxy-4,7,2′,7′-tetrachlorofluorescein); and the 3′ quencher dye TAMRA (6-carboxytetramethylrhodamine), Dabecyl, Black hole quencher 1 (BHQ-1), BHQ-2, Iowa Black FQ and Iowa Black RQ (Livak et al., PCR Methods Appl. 4:357-362, 1995; Gibson et al., Genome Res. 6:995-1001, 1996; Heid et al., Genome Res. 6:986-994, 1996). In addition, a probe may be double quenched by containing an internal quencher such as the ZEN quencher, to provide even greater quenching and reduce background fluorescence further.

In one embodiment, said probe is a hydrolysis probe.

In another embodiment said probes are one or more hydrolysis probes directed to a region of partial cytosine methylation wherein said one or more probes collectively hybridise to at least two differing methylation patterns at said region.

It would be appreciated by the skilled person that to the extent that one is screening for more than one gene marker, one may elect to design the method such that some, but not all, of the gene markers are screened using the method of the present invention while others are screened using standard prior art methylation analysis methods. For example, if two genes are the subject of analysis, one gene may be analysed based on the amplification of both the target strand and the non-complementary opposite strand of the bisulphite (or equivalent) converted DNA (ie by the method of the present invention) while the other gene may undergo standard methylation specific amplification of the target strand alone. This may be appropriate where one of the markers does not require analysis at the same level of sensitivity as the other markers and therefore does not necessarily require the application of the method of the invention. An example of such an analysis design is provided in Example 11.

To the extent that the method of the present invention is designed to detect partially methylated DNA, the probes of the present invention are designed such that they can hybridise, within a single reaction, to a DNA sequence that exhibits at least two different methylation patterns. For example, the probes may hybridise to the fully methylated sequence and to one or more partially methylated sequences. In another example, the probes may detect at least two different partially methylated forms of the DNA sequence. It should be understood that to the extent that the method of the present invention is directed to providing an accurate and reproducible means of detecting the methylation of a DNA target which exhibits both fully and partially methylated forms, this method of detection is designed to focus the probes to one discrete region of the DNA sequence which does, or is thought to, exhibit partially methylated forms. The person of skill in the art would understand, however, that the DNA target may also exhibit partial methylation patterns at regions of the DNA sequence other than the region targeted by the probe. It should therefore be understood that this embodiment of the present method is limited to detecting and assessing partial methylation at the DNA regions to which the probe is directed but not to any other regions of the DNA target. Accordingly, to the extent that one is screening a particular gene target, the method of the present invention is designed to detect all of the partially methylated forms of that gene that exhibit partial methylation at the site to which the probe is directed. However, to the extent that the subject gene may also exhibit partial methylation at other sites along its sequence, these partially methylated forms will not be detected if the probe is not directed to these methylation sites. It would also be appreciated by the skilled person, however, that to the extent that more than one region of potential partial methylation is of interest, the method can be adapted to include the use of probes directed to multiple such regions, provided that these regions are located between the amplification primer pairs.

Reference herein to the subject probe or probes hybridising to at least two “differing methylation patterns at said region” should be understood to mean that the probes that are used in the method of the invention are all designed to hybridise to the same DNA sequence region. However, this DNA sequence region, which is methylated, may exhibit either full methylation or a range of partially methylated forms, this being referred to a “differing methylation patterns” or “differential methylation”. As the number of methylated CpG dinucleotides present in this region increase, the number of potentially different partially methylated patterns increases. For example, in addition to the fully methylated form of IKZF1 at Chr7:50304323-50304349, there are 7 differing methylation patterns between the amplification primers including 6 partially methylated forms and the fully unmethylated form. One may elect to detect all differentially methylated forms of a DNA target of interest, although depending on the circumstances of the situation, one may seek to only screen for some, but not all, the partial methylation forms of a particular DNA target. For example, if it is known that there are two predominant partially methylated forms, one may elect to screen for just these two. It is well within the skill of the person in the art to make this assessment and appropriately design a probe set.

According to this embodiment there is therefore provided a method of screening for the methylation of a gene of interest or gene region thereof, said method comprising:
(i) contacting a DNA sample with a bisulphite agent to convert unmethylated cytosine residues to uracil wherein said sample comprises both the target strand and the opposite strand of said gene region of interest;
(ii) contacting the DNA sample of step (i) with:
   a) a first set of methylation specific forward and reverse primers designed to amplify one or more fully or partially methylated forms of the modified target strand of the gene region of interest;
   b) a second set of methylation specific forward and reverse primers designed to amplify one or more fully or partially methylated form of the modified opposite strand of the DNA region of interest; and
   c) one or more methylation specific probes which incorporate a detection means;
(iii) amplifying the DNA sample of step (ii) wherein if one or more of the probes of step (ii)(c) are used, the extension of said primers along said gene effects the detection of said hybridised probe; and
(iv) qualitatively or quantitatively analysing the detection output of step (iii).

In yet another embodiment, said gene is IKZF1 and:

(i) said first set of primers comprise the sequences:

(FWD PRIMER): Chr7 (+); 50,304,271-50,304,294
SEQ ID NO: 11
5′GACGACGTATTTTTTTCGTGTTTC-3′
(REV PRIMER): Chr7 (−); 50,304,350-50,304,365
SEQ ID NO: 12
5′-GCGCACCTCTCGACCG-3′

or substantially similar sequences and the probes directed to the amplification product of said first set of primers include one or more of the sequences selected from:

SEQ ID NO: 13
5′-TTTGTATYGGAGTAGYGATTYGGGAGG-3′
SEQ ID NO: 14
5′-TTTGTATCGGAGTAGCGATTCGGGAGG-3′
SEQ ID NO: 15
5′-TTTGTATCGGAGTAGCGATTTGGGAGG-3′
SEQ ID NO: 16
5′-TTTGTATCGGAGTAGTGATTCGGGAGG-3′
SEQ ID NO: 17
5′-TTTGTATTGGAGTAGCGATTCGGGAGG-3′
SEQ ID NO: 18
5′-TTTGTATCGGAGTAGTGATTTGGGAGG-3′
SEQ ID NO: 19
5′-TTTGTATTGGAGTAGCGATTTGGGAGG-3′
SEQ ID NO: 20
5′-TTTGTATTGGAGTAGTGATTCGGGAGG-3′
SEQ ID NO: 21
5′-TTTGTATTGGAGTAGTGATTTGGGAGG-3′

or substantially similar sequences; and

(ii) said second set of primers comprise the sequences:

(FWD PRIMER): Chr7 (−); 50,304,295-50,304,314
SEQ ID NO: 77
5′-CGCGTAGAAGGGCGTAGAGC-3′
(REV PRIMER): Chr7 (+); 50,304,234-50,304,254
SEQ ID NO: 78
5′- GCGCGAACCGAAAAACTCGAC-3′

or substantially similar sequences and the probes directed to the amplification product of said second set of primers include one or more of the sequences selected from:

SEQ ID NO: 79
5′-AAYGAYGCACCCTCTCYGTATCCY-3′
SEQ ID NO: 80
5′-AACGACGCACCCTCTCCGTATCCT-3′
SEQ ID NO: 81
5′-AATGACGCACCCTCTCCGTATCCC-3′
SEQ ID NO: 82
5′-AACGATGCACCCTCTCCGTATCCC-3′
SEQ ID NO: 83
5′-AACGACGCACCCTCTCTGTATCCC-3′
SEQ ID NO: 84
5′-AATGATGCACCCTCTCCGTATCCC-3′
SEQ ID NO: 85
5′-AATGACGCACCCTCTCTGTATCCC-3′
SEQ ID NO: 86
5′-AATGACGCACCCTCTCCGTATCCT-3′
SEQ ID NO: 87
5′-AACGATGCACCCTCTCTGTATCCC-3′
SEQ ID NO: 88
5′-AACGATGCACCCTCTCCGTATCCT-3′
SEQ ID NO: 89
5′-AACGACGCACCCTCTCTGTATCCT-3′
SEQ ID NO: 90
5′-AATGATGCACCCTCTCTGTATCCC-3′
SEQ ID NO: 91
5′-AATGACGCACCCTCTCTGTATCCT-3′
SEQ ID NO: 92
5′-AACGATGCACCCTCTCTGTATCCT-3′
SEQ ID NO: 93
5′-AATGATGCACCCTCTCTGTATCCT-3′
SEQ ID NO: 94
5′-AACGACGCACCCTCTCCGTATCCC-3′
SEQ ID NO: 95
5′-AATGATGCACCCTCTCCGTATCCT-3′

or substantially similar sequences.

In still another embodiment, said gene is IKZF1 and:

(i) said first set of primers comprise the sequences:

(FWD PRIMER): Chr7 (+); 50,304,271-50,304,294
SEQ ID NO: 11
5′GACGACGTATTTTTTTCGTGTTTC-3′
(REV PRIMER): Chr7 (−); 50,304,350-50,304,365
SEQ ID NO: 12
5′-GCGCACCTCTCGACCG-3′

or substantially similar sequences and the probes directed to the amplification product of said first set of primers include one or more of the sequences selected from:

SEQ ID NO: 13
5′-TTTGTATYGGAGTAGYGATTYGGGAGG-3′
SEQ ID NO: 14
5′-TTTGTATCGGAGTAGCGATTCGGGAGG-3′
SEQ ID NO: 15
5′-TTTGTATCGGAGTAGCGATTTGGGAGG-3′
SEQ ID NO: 16
5′-TTTGTATCGGAGTAGTGATTCGGGAGG-3′
SEQ ID NO: 17
5′-TTTGTATTGGAGTAGCGATTCGGGAGG-3′
SEQ ID NO: 18
5′-TTTGTATCGGAGTAGTGATTTGGGAGG-3′
SEQ ID NO: 19
5′-TTTGTATTGGAGTAGCGATTTGGGAGG-3′
SEQ ID NO: 20
5′-TTTGTATTGGAGTAGTGATTCGGGAGG-3′
SEQ ID NO: 21
5′-TTTGTATTGGAGTAGTGATTTGGGAGG-3′

or substantially similar sequences; and

(ii) said second set of primers comprise the sequences:

(FWD PRIMER): Chr7 (−); 50,304,366-50,304,391
SEQ ID NO: 22
5′ TTGTTTCGTAGTCGGTTCGGTTTCG 3′
(REV PRIMER): Chr7 (+); 50,304,271-50,304,294
SEQ ID NO: 23
5′-AACGACGCACCCTCTCCGTATCCC-3′

or substantially similar sequences and the probes directed to the amplification product of said second set of primers include one or more of the sequences selected from:

SEQ ID NO: 24
5′-TTTTTYGGATYGTTGTTTYGGTATAGG-3′
SEQ ID NO: 25
5′-TTTTTCGGATCGTTGTTTCGGTATAGG-3′
SEQ ID NO: 26
5′-TTTTTCGGATCGTTGTTTTGGTATAGG-3′
SEQ ID NO: 27
5′-TTTTTCGGATTGTTGTTTCGGTATAGG-3′
SEQ ID NO: 28
5′-TTTTTTGGATCGTTGTTTCGGTATAGG-3′
SEQ ID NO: 29
5′-TTTTTCGGATTGTTGTTTTGGTATAGG-3′
SEQ ID NO: 30
5′-TTTTTTGGATTGTTGTTTCGGTATAGG-3′
SEQ ID NO: 31
5′-TTTTTTGGATCGTTGTTTTGGTATAGG-3′
SEQ ID NO: 32
5′-TTTTTTGGATTGTTGTTTTGGTATAGG-3′

or substantially similar sequences.

In yet still another embodiment, said gene is IKZF1 and:

(i) said first set of primers comprise the sequences:

SEQ ID NO: 11
(FWD PRIMER): Chr7 (+); 50,304,271-50,304,294
5′ GACGACGTATTTTTTTCGTGTTTC-3′
SEQ ID NO: 12
(REV PRIMER): Chr7 (−); 50,304,350-50,304,365
5′-GCGCACCTCTCGACCG-3′

or substantially similar sequences and the probes directed to the amplification product of said first set of include one or more of the sequences selected from:

SEQ ID NO: 13
5′-TTTGTATYGGAGTAGYGATTYGGGAGG-3′
SEQ ID NO: 14
5′-TTTGTATCGGAGTAGCGATTCGGGAGG-3′
SEQ ID NO: 15
5′-TTTGTATCGGAGTAGCGATTTGGGAGG-3′
SEQ ID NO: 16
5′-TTTGTATCGGAGTAGTGATTCGGGAGG-3′
SEQ ID NO: 17
5′-TTTGTATTGGAGTAGCGATTCGGGAGG-3′
SEQ ID NO: 18
5′-TTTGTATCGGAGTAGTGATTTGGGAGG-3′
SEQ ID NO: 19
5′-TTTGTATTGGAGTAGCGATTTGGGAGG-3′
SEQ ID NO: 20
5′-TTTGTATTGGAGTAGTGATTCGGGAGG-3′
SEQ ID NO: 21
5′-TTTGTATTGGAGTAGTGATTTGGGAGG-3′

or substantially similar sequences; and

(ii) said second set of primers comprise the sequences:

(FWD PRIMER): Chr7 (−); 50,304,329-50,304,355
SEQ ID NO: 33
5′-CGGTCGTTTTTCGGATCGTTGTTTCGG-3′
(REV PRIMER): Chr7 (+); 50,304,271-50,304,294
SEQ ID NO: 23
5′-AACGACGCACCCTCTCCGTATCCC-3′

or substantially similar sequences and the probes directed to the amplification product of said second set of primers include one or more of the sequences selected from:

SEQ ID NO: 34
5′-YGYGTAGAAGGGYGTAGAGYG-3′
SEQ ID NO: 35
5′-CGCGTAGAAGGGCGTAGAGCG-3′
SEQ ID NO: 36
5′-CGCGTAGAAGGGCGTAGAGTG-3′
SEQ ID NO: 37
5′-CGCGTAGAAGGGTGTAGAGTG-3′
SEQ ID NO: 38
5′-CGTGTAGAAGGGTGTAGAGTG-3′
SEQ ID NO: 39
5′-TGTGTAGAAGGGTGTAGAGTG-3′
SEQ ID NO: 40
5′-TGCGTAGAAGGGCGTAGAGCG-3′
SEQ ID NO: 41
5′-CGTGTAGAAGGGCGTAGAGCG-3′
SEQ ID NO: 42
5′-CGCGTAGAAGGGTGTAGAGCG-3′
SEQ ID NO: 43
5′-TGTGTAGAAGGGCGTAGAGCG-3′
SEQ ID NO: 44
5′-TGCGTAGAAGGGTGTAGAGCG-3′
SEQ ID NO: 45
5′-TGCGTAGAAGGGCGTAGAGTG-3′
SEQ ID NO: 46
5′-TGTGTAGAAGGGTGTAGAGCG-3′
SEQ ID NO: 47
5′-TGTGTAGAAGGGCGTAGAGTG-3′
SEQ ID NO: 48
5′-TGCGTAGAAGGGTGTAGAGTG-3′
SEQ ID NO: 49
5′-CGTGTAGAAGGGCGTAGAGTG-3′
SEQ ID NO: 50
5′-CGTGTAGAAGGGTGTAGAGCG-3′

or substantially similar sequences.

In yet still another embodiment, said gene is BCAT1 and:

(i) said first set of primers comprise the sequences:

(FWD PRIMER): chr12 (+); 24,949,138-24,949,164
SEQ ID NO: 97
5′-TTAGTGTTTTTTTGTTGATGTAATTCG-3′
(REV PRIMER): chr12 (−); 24,949,058-24,949,074
SEQ ID NO: 65
5′-CAATACCCGAAACGACGACG-3′

or substantially similar sequences and the probes directed to the amplification product of said first set of primers include the sequence:

SEQ ID NO: 66
5′-TTCGTCGCGAGAGGGTCGGTT-3′

or substantially similar sequences; and

(ii) said second set of primers comprise the sequences:

(FWD PRIMER): chr12 (−); 24,949,058-24,949,082
SEQ ID NO: 96
5′-TAGTGTTCGAGGCGGCGGCGAGTAT-3′
(REV PRIMER): chr12 (+); 24,949,140-24,949,159
SEQ ID NO: 62
5′-ATCTTCCTACTAATACAATCCGCTAAATC-3′

or substantially similar sequences and the probes directed to the amplification product of said second set of primers include the sequence:

SEQ ID NO: 63
5′-GATCGGTTTTTTCGCGGCGGA-3′

or substantially similar sequence.

In still yet another embodiment, said gene is BCAT1 and:

(i) said first set of primers comprise the sequences:

(FWD PRIMER): chr12 (+); 24,949,131-24,949,159
SEQ ID NO: 64
5′-GTTTTTTTGTTGATGTAATTCGTTAGGTC-3′
(REV PRIMER): chr12 (−); 24,949,058-24,949,074
SEQ ID NO: 65
5′-CAATACCCGAAACGACGACG-3′

or substantially similar sequences and the probes directed to the amplification product of said first set of primers includes the sequence:

SEQ ID NO: 66
5′-TTCGTCGCGAGAGGGTCGGTT-3′

or substantially similar sequences; and

(ii) said second set of primers comprise the sequences:

(FWD PRIMER): chr12 (−); 24,949,058-24,949,085
SEQ ID NO: 61
5′-TAGTGTTCGAGGCGGCGGCGAGTATACG-3′
(REV PRIMER): chr12 (+); 24,949,140-24,949,159
SEQ ID NO: 62
5′-ATCTTCCTACTAATACAATCCGCTAAATC-3′

or substantially similar sequences and the probes directed to the amplification product of said second set of primers includes the sequence:

SEQ ID NO: 63
5′-GATCGGTTTTTTCGCGGCGGA-3′

or substantially similar sequence.

In a further embodiment, said gene is IRF4 and:
(i) said first set of primers comprise the sequences:

(FWD PRIMER): Chr6 (+): 391,546-391,564
SEQ ID NO: 108
5′-GGCGCGGGAATTTTATTTC-3′
(REV PRIMER): Chr6 (−): 391,605-391,622
SEQ ID NO: 109
5′-AAACCGACCGAACGAACG-3′

or substantially similar sequences and the probes directed to the amplification product of said first set of primers include the sequence:

SEQ ID NO: 110
5′-AAAACCGACGTAAAAACTAAAAACTACCGCGA-3′

or substantially similar sequences; and
(ii) said second set of primers comprise the sequences:

(FWD PRIMER): Chr6 (−): 391,614-391,636
SEQ ID NO: 111
5′-TAAGTCGAGAGTCGGGGTCGGTC-3′
(REV PRIMER): Chr6 (+): 391,524-391,539
SEQ ID NO: 112
5′-CGACGCGCGAAAAATC-3′

or substantially similar sequences and the probes directed to the amplification product of said second set of primers include one or more of the sequences selected from:

SEQ ID NO: 113
5′-CCAACCTTCACGCCGACCCTAAAACTCG-3′
SEQ ID NO: 115
5′-CCTTCACGCCGACCCTAAAACTCG-3′

or substantially similar sequence.

In another further embodiment, said gene is IRF4 and:

(i) said first set of primers comprise the sequences:

(FWD PRIMER): Chr6 (+): 391,546-391,564
SEQ ID NO: 108
5′-GGCGCGGGAATTTTATTTC-3′
(REV PRIMER): Chr6 (−): 391,605-391,622
SEQ ID NO: 109
5′-AAACCGACCGAACGAACG-3′

or substantially similar sequences and the probes directed to the amplification product of said first set of primers include the sequence:

SEQ ID NO: 110
5′-AAAACCGACGTAAAAACTAAAAACTACCGCGA-3′

or substantially similar sequences; and

(ii) said second set of primers comprise the sequences:

(FWD PRIMER): Chr6 (−): 391,614-391,630
SEQ ID NO: 114
5′-GAGAGTCGGGGTCGGTC-3′
(REV PRIMER): Chr6 (+): 391,524-391,539
SEQ ID NO: 112
5′-CGACGCGCGAAAAATC-3′

or substantially similar sequences and the probes directed to the amplification product of said second set of primers include one or more of the sequences selected from:

SEQ ID NO: 113
5′-CCAACCTTCACGCCGACCCTAAAACTCG-3′
SEQ ID NO: 115
5′-CCTTCACGCCGACCCTAAAACTCG-3′

or substantially similar sequence.

In still another further embodiment, said gene is IRF4 and:

(i) said first set of primers comprise the sequences:

(FWD PRIMER): Chr6 (+): 391,546-391,564
SEQ ID NO: 108
5′-GGCGCGGGAATTTTATTTC-3′
(REV PRIMER): Chr6 (−): 391,605-391,622
SEQ ID NO: 109
5′-AAACCGACCGAACGAACG-3″

or substantially similar sequences and the probes directed to the amplification product of said first set of primers include the sequence:

SEQ ID NO: 110
5′-AAAACCGACGTAAAAACTAAAAACTACCGCGA-3′

or substantially similar sequences; and

(ii) said second set of primers comprise the sequences:

(FWD PRIMER): Chr6 (−): 391,624-391,649
SEQ ID NO: 116
5′-GAGAGGGATTTTGTAAGTCGAGAGTC-3′
(REV PRIMER): Chr6 (+): 391,524-391,539
SEQ ID NO: 112
5′-CGACGCGCGAAAAATC-3′

or substantially similar sequences and the probes directed to the amplification product of said second set of primers include one or more of the sequences selected from:

SEQ ID NO: 113
5′-CCAACCTTCACGCCGACCCTAAAACTCG-3′
SEQ ID NO: 115
5′-CCTTCACGCCGACCCTAAAACTCG-3′

or substantially similar sequence.

To the extent that the method of the present invention utilises more than one probe directed to the target and/or the opposite amplification product, it should be understood that the subject primers may correspond to the sequences disclosed above or may be substantially similar. Alternatively, these sequences or a substantially similar sequence may represent a subregion within a larger primer molecule. Reference to a “substantially similar sequence” should be understood as a reference to a sequence which may exhibit some minor difference in sequence but which nevertheless functions to amplify the same DNA target as the sequence to which it is substantially similar.

The probes of the present invention “collectively” bind to the range of partially and fully methylated sequences that are sought to be detected. By “collectively” is meant that the cohort of probes that is selected for use are able, either individually or by virtue of the promiscuity of hybridisation of an individual probe, to bind to the range of partially methylated forms of the DNA target that are sought to be detected. Without limiting the present invention to any one theory or mode of action, the sequence of the DNA region that is to be interrogated by the probe will be known to the skilled person, as will the position of the methylated CpG dinucleotides. Based on this sequence information, and as exemplified earlier in relation to IKZF1, the full range of possible full and partial methylation patterns on both the target and opposite strands can be predicted. Probes can then be designed that either each individually bind to a unique methylation pattern or that exhibit promiscuity and can bind to more than one methylation pattern. A probe directed to a fully methylated sequence will not bind to a partially methylated sequence, even where the difference between the fully methylated sequence and the partially methylated sequence is as little as the lack of methylation of one cytosine residue. It has also been determined that if either a heterogeneous pool of methylation specific probes or probes which are designed to bind promiscuously across both methylated and non-methylated cytosines are used in an amplification assay, an accurate result can be obtained in relation to the methylation of the target of interest.

The probes that are designed to hybridise to one specific fully or partially methylated sequence pattern can be generated by methods which are well known to those of skill in the art. In relation to the probes that exhibit promiscuity, in that they can bind to more than one methylation pattern, this design can also be achieved by several methods which are known to those of skill in the art. For example, one or more base positions in the probe (such as in a 5′-hydrolysis probe) are not unique, but are a mixture of two bases, namely cytosine or thymidine. If only one CpG site is interrogated for methylation (or not) then such degenerate oligonucleotide would be a mixture of two different oligonucleotide sequences, e.g. —atCGat— and —atTGat—. If two CpG sites were interrogated, then the degenerate oligonucleotide cocktail would be a mixture of four different sequences.

As detailed earlier, the probes can be any variance of detection probes such as TaqMan, Scorpions, Beacons, etc. The probe mixture may be synthesised (in the context of the target strand of the IKZF1 example) as

(i) 8-fold redundant in one synthesis (by blending C and T during synthesis);
(ii) three different two-fold redundant probes and mixed;
(iii) one two-fold and one four-fold redundant probes and mixed; or
(iv) eight different unique probes and mixed.

The probe could also be a single sequence with either an abasic spacer (e.g. 5-nitro-indole or 3-nitro-pyrrole) at each interrogated C/T base, or with an Inosine at each interrogated C/T base. A single sequence “promiscuous” probe containing one or more abasic spacer(s) would have only one annealing temperature, but the melting temperature of the abasic spacer(s) containing probe would be significantly lower than the probe detecting methylation on all interrogated CpG sites. Thus a promiscuous probe with abasic spacer(s) would need to be significantly longer than the probe targeting methylated CpG sites only. Inosine will allow base-pairing with any base, but has a preference in the order C>A>G>T. As this sequence is in the opposite strand to the probe, the probe would be annealing to A (=T, unmethylated) or G (=C, methylated) in this case. Both these options are less specific than the promiscuous probe. Because they allow pairing to one of 4 bases at 3 positions, they are in fact 64-fold degenerate (vs 8-fold), and thus rely more heavily on the methylation specificity of the primers. Abasic-spacer or Inosine-containing probes have the benefit of being a single oligonucleotide component, rather than a mixture of 8 oligonucleotide components.

The probe could also have a pyrimidine (C or T) analogue at each potential partially methylated C position. For example, the analogue, 6H,8H-3,4-dihydropyrimido[4,5-c][1,2]oxazin-7-one, is a single “base” that will base pair with both G and A (which are the two options in the opposite strand). From one study it has a 60% preference for A (=T=unmethylated) and 40% for G (=C=methylated). (Hill et al., Proc Nall Acad Sci USA., 95:4258-4263, 1998). The benefit here is that this probe is a single oligonucleotide that will bind all 8 possible methylation combinations with approximately equal affinity. It would be appreciated that since some of the individual probe sequences will contain thymidine instead of cytosine bases, which lowers the annealing temperature, some of the probe sequence(s) may need to be extended in length to compensate for the lower annealing temperature. An alternate approach would be to include chemical modifications that increase annealing temperature (such as major groove binding bases). It should also be understood that the proportions of each base at the degenerate position(s) of the probe do not necessarily have to be 50/50. For example if one identified that a specific C residue was methylated in 80% of true cancer cases but not methylated in 20% of true cancer cases, one could make a probe with 80% C and 20% T at this position to match the incidence of methylation.

As detailed hereinbefore the probe sequence(s) are designed to hybridise to the opposite strand as well. These probe sequence designs on the opposite strand would have a G or an A at the degenerate position (or Inosine or abasic spacer as above) to interrogate partial methylation. The pyrimidine analogue mentioned above would now change to a purine analogue, N6-methoxy-2,6-diaminopurine, that will bind both T and C.

It would be appreciated that these principles can be used to design amplification probes for any gene.

The probes and/or primers of the present invention are also assessed to determine that they do not self-prime or form primer dimers (e.g. with another probe or primer used in a detection assay). Furthermore, a probe or primer (or the sequence thereof) is often assessed to determine the temperature at which it denatures from a target nucleic acid (i.e. the melting temperature of the probe or primer, or Tm). Methods for estimating Tm are known in the art and described, for example, in Santa Lucia, Proc. Natl. Acad. Sci. USA, 95: 1460-1465, 1995 or Breslauer et al., Proc. Natl. Acad. Sci. USA, 83: 3746-3750, 1986.

Methods for producing/synthesizing a probe or primer of the present invention are known in the art. For example, oligonucleotide synthesis is described, in Gait (Ed) (In: Oligonucleotide Synthesis: A Practical Approach, IRL Press, Oxford, 1984). For example, a probe or primer may be obtained by biological synthesis (e.g. by digestion of a nucleic acid with a restriction endonuclease) or by chemical synthesis. For short sequences (up to about 100 nucleotides) chemical synthesis is preferable.

For longer sequences standard replication methods employed in molecular biology are useful, such as, for example, the use of M13 for single stranded DNA as described by Messing, Methods Enzymol, 101, 20-78, 1983. Other methods for oligonucleotide synthesis include, for example, phosphotriester and phosphodiester methods (Narang, et al. Meth. Enzymol 68: 90, 1979) and synthesis on a support (Beaucage, et al. Tetrahedron Letters 22: 1859-1862, 1981) as well as phosphoramidate technique, Caruthers, M. H., et al., “Methods in Enzymology,” Vol. 154, pp. 287-314 (1988), and others described in “Synthesis and Applications of DNA and RNA,” S. A. Narang, editor, Academic Press, New York, 1987, and the references cited therein. Probes comprising locked nucleic acid (LNA) are synthesized as described, for example, in Nielsen et al., J. Chem. Soc. Perkin Trans., 1:3423, 1997; Singh and Wengel, Chem. Commun. 1247, 1998. While, probes comprising peptide-nucleic acid (PNA) are synthesized as described, for example, in Egholm et al., Am. Chem. Soc., 114: 1895, 1992; Egholm et al., Nature, 365: 566, 1993; and Orum et al., Nucl. Acids Res., 21: 5332, 1993.

The DNA sample of the present invention is amplified using primers that flank the region of methylation of interest. As detailed hereinbefore, this “region” may be selected to encompass a small or a substantial part of the length of the gene. In the latter case the amplicons that are generated would be quite long. However, in a particular embodiment, the region may correspond to a much shorter stretch of the gene where one or more CpG dinucleotides are clustered. In this case the amplicons that are generated would be significantly shorter.

Facilitating the interaction of the primers and probes with the target DNA may be performed by any suitable method. Those methods will be known to those skilled in the art. To this end, it should be understood that the primers and probes can be incorporated into the reaction tube at any suitable time point. While incorporation is generally prior to the commencement of the initial amplification cycles, incorporation of one or more additional primers may be performed subsequently to the initial amplification cycles. The mode of incorporation of the primers will depend on how the skilled person is seeking to perform the amplification reaction but, in general, for ease of use and avoidance of contamination, it is usually desirable to be able to perform the entire reaction in a single tube. Nevertheless, any other method of achieving the steps of the invention can be used. Accordingly, reference to “contacting” the sample with the primer or probe oligonucleotide should be understood as a reference to facilitating the mixing of the primer or probe with the sample such that interaction (for example, hybridisation) can occur. Means of achieving this objective would be well known to those of skill in the art.

Where multiple methylated DNA regions are to be amplified, the skilled person may design multiplexed amplification reactions. This multiplexing may be designed at the level of amplifying both the target and the opposite strands in the one tube or, alternatively, amplifying multiple regions of either the target or opposite strand in a single tube. For example, where more than one pair of forward/reverse primers are used, directed to targeting two or more separate gene or methylation regions, one may introduce all these primers to a single sample and amplify the sample using a multiplexed amplification technique. Alternatively, one may elect to divide the sample into more than one aliquot wherein each aliquot is amplified using a separate pair of primers. It should also be understood that the skilled person may elect to adapt this method so as to use multiple sets of primers, directed to amplifying only one methylation region but where the multiple primers reflect the application of a nested PCR reaction. Alternatively, several individual amplification reactions that each use one unique primer pair may be performed. These methods become relevant where one is amplifying two or more separate methylation regions, where the methylation of more than one gene is to be analysed or where one seeks to separate the amplification reactions of the target and opposite strands. In this case, one may divide the sample into two aliquots, for example, after the sodium bisulphite conversion, if two genes are sought to be analysed (such as BCAT1 and IKZF1), with each aliquot then being amplified using the one or more sets of forward and reverse primers directed to the relevant methylation sequence regions of that gene. Alternatively, a multiplexed reaction can be performed on a single sample wherein the reaction is multiplexed in terms of the use of a primer pair and hydrolysis probe set directed to a selected methylation sequence region of one gene and the use of another set of primers and a hydrolysis probe set directed to a selected methylation sequence region of another gene. As would be familiar to the skilled person, multiplexed reactions can be designed to be performed with two, three or more sets of primers and hydrolysis probes in the context of two or more methylation sequence regions and/or two or more genes or both the target and the opposite strand. It should be understood that it would be well within the skill of the person in the art to appropriately design multiplexed or nested amplification reactions.

The amplification step of the present invention leads to extension of the hybridised primers along the DNA target of interest. As detailed hereinbefore it is the generation of the primer extension molecule that effects the detection of the hybridised dual-labelled hydrolysis probe. The means by which this can be effected would be well known to the skilled person as would the fact that the detection means output, which is generated upon amplicon production, can be analysed either qualitatively or quantitatively, the latter being a particularly preferred means. To this end, it should be understood that the detection of the probe is only effected when the primers extend along the DNA sequence to which the probe is hybridised and displace, cleave or otherwise effect a modification to the probe which enables its detection means to become functional (e.g. activated or revealed) and thereby detectable by either qualitative or quantitative means.

Although the preferred application of this method is to assess methylation levels for the purpose of diagnosing disease onset (such as neoplasia development or predisposition thereto), the detection of converse changes in the levels of said methylation may be desired under certain circumstances, for example, to monitor the effectiveness of therapeutic or prophylactic treatment directed to modulating a neoplastic condition, such as adenoma or adenocarcinoma development. For example, where elevated levels of methylation indicate that an individual has developed a condition characterised by adenoma or adenocarcinoma development, screening for a decrease in the levels of methylation subsequently to the onset of a therapeutic treatment regime may be utilised to indicate successful clearance of the neoplastic cells. In another example, one can use this method to test the tissue at the margins of a tumour resection in order to determine whether the full margin of the tumour has been removed.

The present method can therefore be used in the diagnosis, prognosis, classification, prediction of disease risk, detection of recurrence of disease, selection of treatment of a number of types of neoplasms and monitoring of neoplasms. A cancer at any stage of progression can be detected, such as primary, metastatic, and recurrent cancers. Still further, this method has applications in any other context where analysis of DNA and RNA methylation is necessitated.

Using neoplasm development as a non-limiting example, the present invention provides methods for determining whether a mammal (e.g., a human) has neoplasia, whether a biological sample taken from a mammal contains neoplastic cells or DNA derived from neoplastic cells, estimating the risk or likelihood of a mammal developing a neoplasm, monitoring the efficacy of anti-cancer treatment, or selecting the appropriate anti-cancer treatment in a mammal with cancer. Such methods are based on the determination that many neoplastic cells have a different methylation status than normal cells.

The method of the invention can be used to evaluate individuals known or suspected to have neoplasia, or as a routine clinical test, i.e., in an individual not necessarily suspected to have a neoplasia. Further diagnostic assays can be performed to confirm the status of neoplasia in the individual.

Further, the present methods may be used to assess the efficacy of a course of treatment. For example, the efficacy of an anti-cancer treatment can be assessed by monitoring DNA methylation over time in a mammal having cancer. For example, a reduction or absence of methylation in any of the relevant diagnostic sequences in a biological sample taken from a mammal following a treatment, compared to a level in a sample taken from the mammal before, or earlier in, the treatment, indicates efficacious treatment.

The method of the present invention is therefore useful as a one-time test or as an on-going monitor of those individuals thought to be at risk of disease development or as a monitor of the effectiveness of therapeutic or prophylactic treatment regimes. In these situations, mapping the modulation of methylation levels in any one or more classes of biological samples is a valuable indicator of the status of an individual or the effectiveness of a therapeutic or prophylactic regime that is currently in use. Accordingly, the method of the present invention should be understood to extend to monitoring for increases or decreases in methylation levels in an individual relative to their normal level, or relative to one or more earlier methylation levels determined from a biological sample of said individual.

Another aspect of the present invention is directed to a method of diagnosing or monitoring a condition in a patient, which condition is characterised by modulation of the methylation of a DNA region of interest, said method comprising:
(i) contacting a DNA sample with an agent which modifies unmethylated cytosine residues wherein said sample comprises both the target strand and the opposite strand of said DNA region of interest, which DNA sample comprises a low copy number of said DNA region of interest;
(ii) contacting the DNA sample of step (i) with:
   a) a first set of forward and reverse primers designed to amplify one or more fully or partially methylated forms of the modified target strand of the DNA region of interest;
   b) a second set of forward and reverse primers designed to amplify one or more fully or partially methylated forms of the modified opposite strand of the DNA region of interest; and
   c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said gene effects the detection of said hybridised probe; and
(iv) qualitatively or quantitatively analysing the detection output of step (iii).

In one embodiment, said DNA region of interest is a gene target or region thereof.

In another embodiment, said gene target is ccfDNA, such as disease specific ccfDNA of any one or more of:

| | | | |
|---|---|---|---|
| (1) GRASP | (18) ANGPT2 | (35) NKX2-6 | (52) HOXA5 |
| (2) IRX1 | (19) LHX6 | (36) PAX1 | (53) GDNF |
| (3) SOX21 | (20) NEUROD1 | (37) FOXD2 | (54) FAT4 |
| (4) FGF5 | (21) AC149644.1 | (38) SLC6A15 | (55) HOXA2 |
| (5) ZNF471 | (22) CCDC48 | (39) PHC2 | (56) LPHN3 |
| (6) SUSD5 | (23) EVX1 | (40) FLRT2 | (57) ADCYAP1 |
| (7) FOXB1 | (24) GHSR | (41) GATA2 | (58) GRIA2 |
| (8) PDX1 | (25) HSD17B14 | (42) ADCY8 | (59) AQP1 |
| (9) DLX5 | (26) KRBA1 | (43) CNNM1 | (60) BCAT1 |
| (10) ONECUT2 | (27) OTOP1 | (44) IKZF1 | (61) CYP24A1 |
| (11) DMRTA2 | (28) PPYR1 | (45) NKX2-3 | (62) FOXI2 |
| (12) CMTM2 | (29) SRMS | (46) PCDH7 | (63) GSX1 |
| (13) OTX2 | (30) ZNF582 | (47) SNCB | (64) IRF4 |
| (14) LOC145845 | (31) IRX2 | (48) ST8SIA1 | (65) NPY |
| (15) EBF3 | (32) CSMD1 | (49) TRAPPC9 | (66) PDE1B |
| (16) SALL1 | (33) MIR675, | (50) NKX2-2 | (67) CAHM |
| | H19 | | |
| (17) CBX8 | (34) FOXD3 | (51) SLC32Al | (68) SEPTIN9 |
| (69) BMP3 | (70) NDRG4 | (71) SDC2 | (72) ZCAN18 |
| (73) COL4A2 | (74) FOXF1 | (75) SOX21 | (76) SLC6A15 |
| (77) ST8SIAI | (78) FGF5 | (79) FOXI2 | |

In yet still another embodiment, said gene is one or more of BCAT1, IKZF1, IRF4, GRASP or CAHM, in particular BCAT1 and/or IKZF1.

In yet another embodiment, the result of step (iv) exhibits a reduced incidence of false negative results.

In still another embodiment, said DNA sample is blood, plasma, serum, saliva, stool, ascites fluid or urine.

In still further embodiment, said DNA region of interest is ccfDNA, such as disease specific ccfDNA, in particular ctDNA.

In yet another embodiment, said primers and probes are methylation specific.

In still yet another embodiment, said primers are not methylation specific but said probes are methylation specific.

In a still further embodiment said probes are one or more hydrolysis probes directed to a region of partial cytosine methylation wherein said one or more probes collectively hybridise to at least two differing methylation patterns at said region.

In one embodiment, said low copy number is less than 100 copies of target DNA/sample tested. In another embodiment said low copy number is less than 95 copies of target DNA/sample tested, less than 90 copies of target DNA/sample tested, less than 85 copies of target DNA/sample tested, less than 80 copies of target DNA/sample tested, less than 75 copies of target DNA/sample tested, less than 70 copies of target DNA/sample tested, less than 65 copies of target DNA/sample tested, less than 60 copies of target DNA/sample tested, less than 55 copies of target DNA/sample tested, less than 50 copies of target DNA/sample tested, less than 45 copies of target DNA/sample tested, less than 40 copies of target DNA/sample tested, less than 35 copies of target DNA/sample tested, less than 30 copies of target DNA/sample tested, less than 25 copies of target DNA/sample tested, less than 20 copies of target DNA/sample tested, less than 15 copies of target DNA/sample tested, less than 10 copies of target DNA/sample tested or less than 5 copies of target DNA/sample tested. Most particularly said low copy number is less than 50 copies of target DNA/sample tested, still more particularly less than 40 copies of target DNA/sample tested, yet more particularly less than 30 copies of target DNA/sample tested, still more particularly less than 20 copies of target DNA/sample tested. In a further embodiment, said low copy number is less than 10 copies of target DNA/sample tested, most particularly less than 5 copies of target DNA/sample tested.

In another embodiment, said amplification is quantitative PCR and said low copy number is the LOD.

In yet another embodiment, said gene is IKZF1 and:

(i) said first set of primers comprise the sequences:

(FWD PRIMER): Chr7 (+); 50,304,271-50,304,294
SEQ ID NO: 11
5′ GACGACGTATTTTTTTCGTGTTTC-3′
(REV PRIMER): Chr7 (−); 50,304,350-50,304,365
SEQ ID NO: 12
5′-GCGCACCTCTCGACCG-3′

or substantially similar sequences and the probes directed to the amplification product of said first set of primers include one or more of the sequences selected from:

SEQ ID NO: 13
5′-TTTGTATYGGAGTAGYGATTYGGGAGG-3′
SEQ ID NO: 14
5′-TTTGTATCGGAGTAGCGATTCGGGAGG-3′
SEQ ID NO: 15
5′-TTTGTATCGGAGTAGCGATTTGGGAGG-3′
SEQ ID NO: 16
5′-TTTGTATCGGAGTAGTGATTCGGGAGG-3′
SEQ ID NO: 17
5′-TTTGTATTGGAGTAGCGATTCGGGAGG-3′
SEQ ID NO: 18
5′-TTTGTATCGGAGTAGTGATTTGGGAGG-3′
SEQ ID NO: 19
5′-TTTGTATTGGAGTAGCGATTTGGGAGG-3′
SEQ ID NO: 20
5′-TTTGTATTGGAGTAGTGATTCGGGAGG-3′
SEQ ID NO: 21
5′-TTTGTATTGGAGTAGTGATTTGGGAGG-3′

or substantially similar sequences; and

(ii) said second set of primers comprise the sequences:

(FWD PRIMER): Chr7 (−); 50,304,295-50,304,314
SEQ ID NO: 77
5′-CGCGTAGAAGGGCGTAGAGC-3′
(REV PRIMER): Chr7 (+); 50,304,234-50,304,254
SEQ ID NO: 78
5′-GCGCGAACCGAAAAACTCGAC-3′

or substantially similar sequences and the probes directed to the amplification product of said second set of primers include one or more of the sequences selected from:

SEQ ID NO: 79
5′-AAYGAYGCACCCTCTCYGTATCCY-3′
SEQ ID NO: 80
5′-AACGACGCACCCTCTCCGTATCCT-3′
SEQ ID NO: 81
5′-AATGACGCACCCTCTCCGTATCCC-3′
SEQ ID NO: 82
5′-AACGATGCACCCTCTCCGTATCCC-3′
SEQ ID NO: 83
5′-AACGACGCACCCTCTCTGTATCCC-3′
SEQ ID NO: 84
5′-AATGATGCACCCTCTCCGTATCCC-3′
SEQ ID NO: 85
5′-AATGACGCACCCTCTCTGTATCCC-3′
SEQ ID NO: 86
5′-AATGACGCACCCTCTCCGTATCCT-3′
SEQ ID NO: 87
5′-AACGATGCACCCTCTCTGTATCCC-3′
SEQ ID NO: 88
5′-AACGATGCACCCTCTCCGTATCCT-3′
SEQ ID NO: 89
5′-AACGACGCACCCTCTCTGTATCCT-3′
SEQ ID NO: 90
5′-AATGATGCACCCTCTCTGTATCCC-3′
SEQ ID NO: 91
5′-AATGACGCACCCTCTCTGTATCCT-3′
SEQ ID NO: 92
5′-AACGATGCACCCTCTCTGTATCCT-3′
SEQ ID NO: 93
5′-AATGATGCACCCTCTCTGTATCCT-3′
SEQ ID NO: 94
5′-AACGACGCACCCTCTCCGTATCCC-3′
SEQ ID NO: 95
5′-AATGATGCACCCTCTCCGTATCCT-3′

or substantially similar sequences.

In still another embodiment, said gene is IKZF1 and:

(i) said first set of primers comprise the sequences:

(FWD PRIMER): Chr7 (+); 50, 304, 271-50, 304, 294
SEQ ID NO: 11
5′GACGACGTATTTTTTTCGTGTTTC-3′
(REV PRIMER): Chr7 (−); 50, 304, 350-50, 304, 365
SEQ ID NO: 12
5′-GCGCACCTCTCGACCG-3′

or substantially similar sequences and the probes directed to the amplification product of said first set of primers include one or more of the sequences selected from:

SEQ ID NO: 13
5′-TTTGTATYGGAGTAGYGATTYGGGAGG-3′
SEQ ID NO: 14
5′-TTTGTATCGGAGTAGCGATTCGGGAGG-3′
SEQ ID NO: 15
5′-TTTGTATCGGAGTAGCGATTTGGGAGG-3′
SEQ ID NO: 16
5′-TTTGTATCGGAGTAGTGATTCGGGAGG-3′
SEQ ID NO: 17
5′-TTTGTATTGGAGTAGCGATTCGGGAGG-3′
SEQ ID NO: 18
5′-TTTGTATCGGAGTAGTGATTTGGGAGG-3′
SEQ ID NO: 19
5′-TTTGTATTGGAGTAGCGATTTGGGAGG-3′
SEQ ID NO: 20
5′-TTTGTATTGGAGTAGTGATTCGGGAGG-3′
SEQ ID NO: 21
5′-TTTGTATTGGAGTAGTGATTTGGGAGG-3′

or substantially similar sequences; and

(ii) said second set of primers comprise the sequences:

(FWD PRIMER): Chr7 (−); 50, 304, 366-50, 304, 391
SEQ ID NO: 22
5′TTGTTTCGTAGTCGGTTCGGTTTCG 3′
(REV PRIMER): Chr7 (+); 50, 304, 271-50, 304, 294
SEQ ID NO: 23
5′-AACGACGCACCCTCTCCGTATCCC-3′

or substantially similar sequences and the probes directed to the amplification product of said second set of primers include one or more of the sequences selected from:

SEQ ID NO: 24
5′-TTTTTYGGATYGTTGTTTYGGTATAGG-3′
SEQ ID NO: 25
5′-TTTTTCGGATCGTTGTTTCGGTATAGG-3′
SEQ ID NO: 26
5′-TTTTTCGGATCGTTGTTTTGGTATAGG-3′
SEQ ID NO: 27
5′-TTTTTCGGATTGTTGTTTCGGTATAGG-3′
SEQ ID NO: 28
5′-TTTTTTGGATCGTTGTTTCGGTATAGG-3′
SEQ ID NO: 29
5′-TTTTTCGGATTGTTGTTTTGGTATAGG-3′
SEQ ID NO: 30
5′-TTTTTTGGATTGTTGTTTCGGTATAGG-3′
SEQ ID NO: 31
5′-TTTTTTGGATCGTTGTTTTGGTATAGG-3′
SEQ ID NO: 32
5′-TTTTTTGGATTGTTGTTTTGGTATAGG-3′

or substantially similar sequences.

In yet still another embodiment, said gene is IKZF1 and:

(i) said first set of primers comprise the sequences:

(FWD PRIMER): Chr7 (+); 50, 304, 271-50, 304, 294
SEQ ID NO: 11
5′GACGACGTATTTTTTTCGTGTTTC-3′
(REV PRIMER): Chr7 (−); 50, 304, 350-50, 304, 365
SEQ ID NO: 12
5′-GCGCACCTCTCGACCG-3′

or substantially similar sequences and the probes directed to the amplification product of said first set of include one or more of the sequences selected from:

SEQ ID NO: 13
5′-TTTGTATYGGAGTAGYGATTYGGGAGG-3′
SEQ ID NO: 14
5′-TTTGTATCGGAGTAGCGATTCGGGAGG-3′
SEQ ID NO: 15
5′-TTTGTATCGGAGTAGCGATTTGGGAGG-3′
SEQ ID NO: 16
5′-TTTGTATCGGAGTAGTGATTCGGGAGG-3′
SEQ ID NO: 17
5′-TTTGTATTGGAGTAGCGATTCGGGAGG-3′
SEQ ID NO: 18
5′-TTTGTATCGGAGTAGTGATTTGGGAGG-3′
SEQ ID NO: 19
5′-TTTGTATTGGAGTAGCGATTTGGGAGG-3′
SEQ ID NO: 20
5′-TTTGTATTGGAGTAGTGATTCGGGAGG-3′
SEQ ID NO: 21
5′-TTTGTATTGGAGTAGTGATTTGGGAGG-3′

or substantially similar sequences; and

(ii) said second set of primers comprise the sequences:

(FWD PRIMER): Chr7 (−); 50, 304, 329-50, 304, 355
SEQ ID NO: 33
5′-CGGTCGTTTTTCGGATCGTTGTTTCGG-3′
(REV PRIMER): Chr7 (+); 50, 304, 271-50, 304, 294
SEQ ID NO: 23
5′-AACGACGCACCCTCTCCGTATCCC-3′

or substantially similar sequences and the probes directed to the amplification product of said second set of primers include one or more of the sequences selected from:

SEQ ID NO: 34
5′-YGYGTAGAAGGGYGTAGAGYG-3′
SEQ ID NO: 35
5′-CGCGTAGAAGGGCGTAGAGCG-3′
SEQ ID NO: 36
5′-CGCGTAGAAGGGCGTAGAGTG-3′
SEQ ID NO: 37
5′-CGCGTAGAAGGGTGTAGAGTG-3′
SEQ ID NO: 38
5′-CGTGTAGAAGGGTGTAGAGTG-3′
SEQ ID NO: 39
5′-TGTGTAGAAGGGTGTAGAGTG-3′
SEQ ID NO: 40
5′-TGCGTAGAAGGGCGTAGAGCG-3′
SEQ ID NO: 41
5′-CGTGTAGAAGGGCGTAGAGCG-3′
SEQ ID NO: 42
5′-CGCGTAGAAGGGTGTAGAGCG-3′
SEQ ID NO: 43
5′-TGTGTAGAAGGGCGTAGAGCG-3′
SEQ ID NO: 44
5′-TGCGTAGAAGGGTGTAGAGCG-3′
SEQ ID NO: 45
5′-TGCGTAGAAGGGCGTAGAGTG-3′
SEQ ID NO: 46
5′-TGTGTAGAAGGGTGTAGAGCG-3′
SEQ ID NO: 47
5′-TGTGTAGAAGGGCGTAGAGTG-3′
SEQ ID NO: 48
5′-TGCGTAGAAGGGTGTAGAGTG-3′
SEQ ID NO: 49
5′-CGTGTAGAAGGGCGTAGAGTG-3′
SEQ ID NO: 50
5′-CGTGTAGAAGGGTGTAGAGCG-3′

or substantially similar sequences.

In yet still another embodiment, said gene is BCAT1 and:

(i) said first set of primers comprise the sequences:

(FWD PRIMER): chr12 (+); 24, 949, 138-24, 949, 164
SEQ ID NO: 97
5′-TTAGTGTTTTTTTGTTGATGTAATTCG-3′
(REV PRIMER): chr12 (−); 24, 949, 058-24, 949, 074
SEQ ID NO: 65
5′-CAATACCCGAAACGACGACG-3′

or substantially similar sequences and the probes directed to the amplification product of said first set of primers include the sequence:

SEQ ID NO: 66
5′-TTCGTCGCGAGAGGGTCGGTT-3′

or substantially similar sequences; and

(ii) said second set of primers comprise the sequences:

(FWD PRIMER): chr12 (−); 24, 949, 058-24, 949,
082
SEQ ID NO: 96
5′-TAGTGTTCGAGGCGGCGGCGAGTAT-3′
(REV PRIMER): chr12 (+); 24, 949, 140-24, 949,
159
SEQ ID NO: 62
5′-ATCTTCCTACTAATACAATCCGCTAAATC-3′

or substantially similar sequences and the probes directed to the amplification product of said second set of primers include the sequence:

SEQ ID NO: 63
5′-GATCGGTTTTTTCGCGGCGGA-3′

or substantially similar sequence.

In still yet another embodiment, said gene is BCAT1 and:

(i) said first set of primers comprise the sequences:

(FWD PRIMER): chr12 (+); 24, 949, 131-24, 949,
159
SEQ ID NO: 64
5′-GTTTTTTTGTTGATGTAATTCGTTAGGTC-3′
(REV PRIMER): chr12 (−); 24, 949, 058-24, 949,
074
SEQ ID NO: 65
5′-CAATACCCGAAACGACGACG-3′

or substantially similar sequences and the probes directed to the amplification product of said first set of primers includes the sequence:

SEQ ID NO: 66
5′-TTCGTCGCGAGAGGGTCGGTT-3′

or substantially similar sequences; and

(ii) said second set of primers comprise the sequences:

(FWD PRIMER): chr12 (−); 24, 949, 058-24, 949,
085
SEQ ID NO: 61
5′-TAGTGTTCGAGGCGGCGGCGAGTATACG-3′
(REV PRIMER): chr12 (+); 24, 949, 140-24, 949,
159
SEQ ID NO: 62
5′-ATCTTCCTACTAATACAATCCGCTAAATC-3′

or substantially similar sequences and the probes directed to the amplification product of said second set of primers includes the sequence:

SEQ ID NO: 63
5′-GATCGGTTTTTTCGCGGCGGA-3′

or substantially similar sequence.

In a further embodiment, said gene is IRF4 and:

(i) said first set of primers comprise the sequences:

(FWD PRIMER): Chr6 (+): 391,546-391,564
SEQ ID NO: 108
5′-GGCGCGGGAATTTTATTTC-3′
(REV PRIMER): Chr6 (−): 391,605-391,622
SEQ ID NO: 109
5′-AAACCGACCGAACGAACG-3′

or substantially similar sequences and the probes directed to the amplification product of said first set of primers include the sequence:

SEQ ID NO: 110
5′-AAAACCGACGTAAAAACTAAAAACTACCGCGA-3′

or substantially similar sequences; and

(ii) said second set of primers comprise the sequences:

(FWD PRIMER): Chr6 (−): 391,614-391,636
SEQ ID NO: 111
5′-TAAGTCGAGAGTCGGGGTCGGTC-3′
(REV PRIMER): Chr6 (+): 391,524-391,539
SEQ ID NO: 112
5′-CGACGCGCGAAAAATC-3′

or substantially similar sequences and the probes directed to the amplification product of said second set of primers include one or more of the sequences selected from:

SEQ ID NO: 113
5′-CCAACCTTCACGCCGACCCTAAAACTCG-3′
SEQ ID NO: 115
5′-CCTTCACGCCGACCCTAAAACTCG-3′

or substantially similar sequence.

In another further embodiment, said gene is IRF4 and:

(i) said first set of primers comprise the sequences:

(FWD PRIMER): Chr6 (+): 391,546-391,564
SEQ ID NO: 108
5′-GGCGCGGGAATTTTATTTC-3′
(REV PRIMER): Chr6 (−): 391,605-391,622
SEQ ID NO: 109
5′-AAACCGACCGAACGAACG-3′

or substantially similar sequences and the probes directed to the amplification product of said first set of primers include the sequence:

SEQ ID NO: 110
5′-AAAACCGACGTAAAAACTAAAAACTACCGCGA-3′

or substantially similar sequences; and

(ii) said second set of primers comprise the sequences:

(FWD PRIMER): Chr6 (−): 391,614-391,630
SEQ ID NO: 114
5′-GAGAGTCGGGGTCGGTC-3′
(REV PRIMER): Chr6 (+): 391,524-391,539
SEQ ID NO: 112
5′-CGACGCGCGAAAAATC-3′

or substantially similar sequences and the probes directed to the amplification product of said second set of primers include one or more of the sequences selected from:

SEQ ID NO: 113
5′-CCAACCTTCACGCCGACCCTAAAACTCG-3′
SEQ ID NO: 115
5′-CCTTCACGCCGACCCTAAAACTCG-3′

or substantially similar sequence.

In still another further embodiment, said gene is IRF4 and:

(i) said first set of primers comprise the sequences:

(FWD PRIMER): Chr6 (+): 391,546-391,564
SEQ ID NO: 108
5′-GGCGCGGGAATTTTATTTC-3′
(REV PRIMER): Chr6 (−): 391,605-391,622
SEQ ID NO: 109
5′-AAACCGACCGAACGAACG-3″

or substantially similar sequences and the probes directed to the amplification product of said first set of primers include the sequence:

SEQ ID NO: 110
5′-AAAACCGACGTAAAAACTAAAAACTACCGCGA-3′

or substantially similar sequences; and

(ii) said second set of primers comprise the sequences:

(FWD PRIMER): Chr6 (−): 391,624-391,649
SEQ ID NO: 116
5′-GAGAGGGATTTTGTAAGTCGAGAGTC-3′
(REV PRIMER): Chr6 (+): 391,524-391,539
SEQ ID NO: 112
5′-CGACGCGCGAAAAATC-3′

or substantially similar sequences and the probes directed to the amplification product of said second set of primers include one or more of the sequences selected from:

SEQ ID NO: 113
5′-CCAACCTTCACGCCGACCCTAAAACTCG-3′
SEQ ID NO: 115
5′-CCTTCACGCCGACCCTAAAACTCG-3′

or substantially similar sequence.

Still another aspect of the present invention is directed a kit for assaying biological samples comprising one or more primers and/or probes for detecting one or more neoplastic markers in accordance with the method of the present invention and reagents useful for facilitating the detection by said primers and/or probes. Further means may also be included, for example, to receive a biological sample.

Accordingly, in another aspect there is provided a kit for screening for the methylation of a DNA region of interest, said kit comprising:
a) a first set of forward and reverse primers designed to amplify one or more fully or partially methylated forms of a target strand of the DNA region of interest, which primers are designed to hybridise to a form of the DNA region of interest which has undergone modification by an agent of unmethylated cytosines;
b) a second set of forward and reverse primers designed to amplify one or more fully or partially methylated forms of the modified opposite strand of the DNA region of interest; and
c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means;

In another embodiment, said DNA region is one or more of the genes:

| | | | |
|---|---|---|---|
| (1) GRASP | (18) ANGPT2 | (35) NKX2-6 | (52) HOXA5 |
| (2) IRX1 | (19) LHX6 | (36) PAX1 | (53) GDNF |
| (3) SOX21 | (20) NEUROD1 | (37) FOXD2 | (54) FAT4 |
| (4) FGF5 | (21) AC149644.1 | (38) SLC6A15 | (55) HOXA2 |
| (5) ZNF471 | (22) CCDC48 | (39) PHC2 | (56) LPHN3 |
| (6) SUSD5 | (23) EVX1 | (40) FLRT2 | (57) ADCYAP1 |
| (7) FOXB1 | (24) GHSR | (41) GATA2 | (58) GRIA2 |
| (8) PDX1 | (25) HSD17B14 | (42) ADCY8 | (59) AQP1 |
| (9) DLX5 | (26) KRBA1 | (43) CNNM1 | (60) BCAT1 |
| (10) ONECUT2 | (27) OTOP1 | (44) IKZF1 | (61) CYP24A1 |
| (11) DMRTA2 | (28) PPYR1 | (45) NKX2-3 | (62) FOXI2 |
| (12) CMTM2 | (29) SRMS | (46) PCDH7 | (63) GSX1 |
| (13) OTX2 | (30) ZNF582 | (47) SNCB | (64) IRF4 |
| (14) LOC145845 | (31) IRX2 | (48) ST8SIA1 | (65) NPY |
| (15) EBF3 | (32) CSMD1 | (49) TRAPPC9 | (66) PDE1B |
| (16) SALL1 | (33) MIR675, | (50) NKX2-2 | (67) CAHM |
| | H19 | | |
| (17) CBX8 | (34) FOXD3 | (51) SLC32A1 | (68) SEPTIN9 |
| (69) BMP3 | (70) NDRG4 | (71) SDC2 | (72) ZCAN18 |
| (73) COL4A2 | (74) FOXF1 | (75) SOX21 | (76) SLC6A15 |
| (77) ST8SIAI | (78) FGF5 | (79) FOXI2 | |

and wherein said gene includes 5 kb upstream of the transcription start site.

In still another embodiment said gene is one or more of the genes BCAT1, IKZF1, IRF4, GRASP or CAHM or 5 kb upstream of the transcription start site.

In yet another embodiment, said genes are selected from (i) BCAT1 and IKZF1; (ii) BCAT1, IKZF1 and IRF4; (iii) BCAT1, IKZF1 and GRASP; (iv) BCAT1, IKZF1 and CAHM; (iv) BCAT1, IKZF1, IRF4 and GRASP; (v) BCAT1, IKZF1, IRF4 and CAHM; or (vi) BCAT1, IKZF1, IRF4, GRASP and CAHM or 5 kb upstream of the transcription start sites of these genes.

In yet still another embodiment said agent modifies unmethylated cytosine residues to uracil and said agent may be a bisulphite salt such as sodium bisulphite or sodium metabisulphite.

In still yet another embodiment, said kit comprises methylation specific primers but no probes.

In a further embodiment, said kit comprises methylation specific primers and non-methylation specific probes.

In yet another further embodiment, said kit comprises methylation specific primers and methylation specific probes.

In still a further embodiment, said kit comprises non-methylation specific primers and methylation specific probes.

In still yet a further embodiment, said probes are hydrolysis probes.

In yet still a further embodiment said probes collectively hybridise to all the full and partial methylation patterns at said DNA region of interest.

In another embodiment, said gene is IKZF1 and:

(i) said first set of primers comprise the sequences:

(FWD PRIMER): Chr7 (+); 50,304,271-50,304,294
SEQ ID NO: 11
5′GACGACGTATTTTTTTCGTGTTTC-3′
(REV PRIMER): Chr7 (−); 50,304,350-50,304,365
SEQ ID NO: 12
5′-GCGCACCTCTCGACCG-3′

or substantially similar sequences and the probes directed to the amplification product of said first set of primers include one or more of the sequences selected from:

SEQ ID NO: 13
5′-TTTGTATYGGAGTAGYGATTYGGGAGG-3′
SEQ ID NO: 14
5′-TTTGTATCGGAGTAGCGATTCGGGAGG-3′
SEQ ID NO: 15
5′-TTTGTATCGGAGTAGCGATTTGGGAGG-3′
SEQ ID NO: 16
5′-TTTGTATCGGAGTAGTGATTCGGGAGG-3′
SEQ ID NO: 17
5′-TTTGTATTGGAGTAGCGATTCGGGAGG-3′
SEQ ID NO: 18
5′-TTTGTATCGGAGTAGTGATTTGGGAGG-3′
SEQ ID NO: 19
5′-TTTGTATTGGAGTAGCGATTTGGGAGG-3′
SEQ ID NO: 20
5′-TTTGTATTGGAGTAGTGATTCGGGAGG-3′
SEQ ID NO: 21
5′-TTTGTATTGGAGTAGTGATTTGGGAGG-3′

or substantially similar sequences; and

(ii) said second set of primers comprise the sequences:

(FWD PRIMER): Chr7 (-); 50, 304, 295-50, 304, 314
SEQ ID NO: 77
5'-CGCGTAGAAGGGCGTAGAGC-3'
(REV PRIMER): Chr7 (+); 50, 304, 234-50, 304, 254
SEQ ID NO: 78
5'-GCGCGAACCGAAAAACTCGAC-3'

or substantially similar sequences and the probes directed to the amplification product of said second set of primers include one or more of the sequences selected from:

SEQ ID NO:79
5'-AAYGAYGCACCCTCTCYGTATCCY-3'
SEQ ID NO:80
5'-AACGACGCACCCTCTCCGTATCCT-3'
SEQ ID NO:81
5'-AATGACGCACCCTCTCCGTATCCC-3'
SEQ ID NO:82
5'-AACGATGCACCCTCTCCGTATCCC-3'
SEQ ID NO:83
5'-AACGACGCACCCTCTCTGTATCCC-3'
SEQ ID NO:84
5'-AATGATGCACCCTCTCCGTATCCC-3'
SEQ ID NO:85
5'-AATGACGCACCCTCTCTGTATCCC-3'
SEQ ID NO:86
5'-AATGACGCACCCTCTCCGTATCCT-3'
SEQ ID NO:87
5'-AACGATGCACCCTCTCTGTATCCC-3'
SEQ ID NO:88
5'-AACGATGCACCCTCTCCGTATCCT-3'
SEQ ID NO:89
5'-AACGACGCACCCTCTCTGTATCCT-3'
SEQ ID NO:90
5'-AATGATGCACCCTCTCTGTATCCC-3'
SEQ ID NO:91
5'-AATGACGCACCCTCTCTGTATCCT-3'
SEQ ID NO:92
5'-AACGATGCACCCTCTCTGTATCCT-3'
SEQ ID NO:93
5'-AATGATGCACCCTCTCTGTATCCT-3'
SEQ ID NO:94
5'-AACGACGCACCCTCTCCGTATCCC-3'
SEQ ID NO:95
5'-AATGATGCACCCTCTCCGTATCCT-3'

or substantially similar sequences.

In yet another embodiment said gene is IKZF1 and:

(i) said first set of primers comprise the sequences:

(FWD PRIMER): Chr7 (+); 50, 304, 271-50, 304, 294
SEQ ID NO: 11
5'GACGACGTATTTTTTTCGTGTTTC-3'
(REV PRIMER): Chr7 (-); 50, 304, 350-50, 304, 365
SEQ ID NO: 12
5'-GCGCACCTCTCGACCG-3'

or substantially similar sequences and the probes directed to the amplification product of said first set of primers include one or more of the sequences selected from:

SEQ ID NO:13
5'-TTTGTATYGGAGTAGYGATTYGGGAGG-3'
SEQ ID NO:14
5'-TTTGTATCGGAGTAGCGATTCGGGAGG-3'
SEQ ID NO:15
5'-TTTGTATCGGAGTAGCGATTTGGGAGG-3'
SEQ ID NO:16
5'-TTTGTATCGGAGTAGTGATTCGGGAGG-3'
SEQ ID NO:17
5'-TTTGTATTGGAGTAGCGATTCGGGAGG-3'
SEQ ID NO:18
5'-TTTGTATCGGAGTAGTGATTTGGGAGG-3'
SEQ ID NO:19
5'-TTTGTATTGGAGTAGCGATTTGGGAGG-3'
SEQ ID NO:20
5'-TTTGTATTGGAGTAGTGATTCGGGAGG-3'
SEQ ID NO:21
5'-TTTGTATTGGAGTAGTGATTTGGGAGG-3'

or substantially similar sequences; and

(ii) said second set of primers comprise the sequences:

(FWD PRIMER): Chr7 (-); 50, 304, 366-50, 304, 391
SEQ ID NO: 22
5'TTGTTTCGTAGTCGGTTCGGTTTCG 3'
(REV PRIMER): Chr7 (+); 50, 304, 271-50, 304, 294
SEQ ID NO: 23
5'-AACGACGCACCCTCTCCGTATCCC-3'

or substantially similar sequences and the probes directed to the amplification product of said second set of primers include one or more of the sequences selected from:

SEQ ID NO: 24
5'-TTTTTYGGATYGTTGTTTYGGTATAGG-3'
SEQ ID NO: 25
5'-TTTTTCGGATCGTTGTTTCGGTATAGG-3'
SEQ ID NO: 26
5'-TTTTTCGGATCGTTGTTTTGGTATAGG-3'
SEQ ID NO: 27
5'-TTTTTCGGATTGTTGTTTCGGTATAGG-3'
SEQ ID NO: 28
5'-TTTTTTGGATCGTTGTTTCGGTATAGG-3'
SEQ ID NO: 29
5'-TTTTTCGGATTGTTGTTTTGGTATAGG-3'
SEQ ID NO: 30
5'-TTTTTTGGATTGTTGTTTCGGTATAGG-3'
SEQ ID NO: 31
5'-TTTTTTGGATCGTTGTTTTGGTATAGG-3'
SEQ ID NO: 32
5'-TTTTTTGGATTGTTGTTTTGGTATAGG-3'

or substantially similar sequences.

In still another embodiment said gene is IKZF1 and:

(i) said first set of primers comprise the sequences:

(FWD PRIMER): Chr7 (+); 50, 304, 271-50, 304, 294
SEQ ID NO: 11
5'GACGACGTATTTTTTTCGTGTTTC-3'
(REV PRIMER): Chr7 (-); 50, 304, 350-50, 304, 365
SEQ ID NO: 12
5'-GCGCACCTCTCGACCG-3'

or substantially similar sequences and the probes directed to the amplification product of said first set of primers include one or more of the sequences selected from:

SEQ ID NO:13
5'-TTTGTATYGGAGTAGYGATTYGGGAGG-3'
SEQ ID NO: 14
5'-TTTGTATCGGAGTAGCGATTCGGGAGG-3'
SEQ ID NO: 15
5'-TTTGTATCGGAGTAGCGATTTGGGAGG-3'
SEQ ID NO: 16
5'-TTTGTATCGGAGTAGTGATTCGGGAGG-3'
SEQ ID NO: 17
5'-TTTGTATTGGAGTAGCGATTCGGGAGG-3'
SEQ ID NO: 18
5'-TTTGTATCGGAGTAGTGATTTGGGAGG-3'
SEQ ID NO: 19
5'-TTTGTATTGGAGTAGCGATTTGGGAGG-3'
SEQ ID NO: 20
5'-TTTGTATTGGAGTAGTGATTCGGGAGG-3'
SEQ ID NO: 21
5'-TTTGTATTGGAGTAGTGATTTGGGAGG-3'

or substantially similar sequences; and

(ii) said second set of primers comprise the sequences:

(FWD PRIMER): Chr7 (-); 50, 304, 329-50, 304, 355
SEQ ID NO: 33
5'-CGGTCGTTTTTCGGATCGTTGTTTCGG-3'
(REV PRIMER): Chr7 (+); 50, 304, 271-50, 304, 294
SEQ ID NO: 23
5'-AACGACGCACCCTCTCCGTATCCC-3'

or substantially similar sequences and the probes directed to the amplification product of said second set of primers include one or more of the sequences selected from:

SEQ ID NO: 34
5'-YGYGTAGAAGGGYGTAGAGYG-3'
SEQ ID NO: 35
5'-CGCGTAGAAGGGCGTAGAGCG-3'
SEQ ID NO: 36
5'-CGCGTAGAAGGGCGTAGAGTG-3'
SEQ ID NO: 37
5'-CGCGTAGAAGGGTGTAGAGTG-3'
SEQ ID NO: 38
5'-CGTGTAGAAGGGTGTAGAGTG-3'
SEQ ID NO: 39
5'-TGTGTAGAAGGGTGTAGAGTG-3'
SEQ ID NO: 40
5'-TGCGTAGAAGGGCGTAGAGCG-3'
SEQ ID NO: 41
5'-CGTGTAGAAGGGCGTAGAGCG-3'
SEQ ID NO: 42
5'-CGCGTAGAAGGGTGTAGAGCG-3'
SEQ ID NO: 43
5'-TGTGTAGAAGGGCGTAGAGCG-3'
SEQ ID NO: 44
5'-TGCGTAGAAGGGTGTAGAGCG-3'
SEQ ID NO: 45
5'-TGCGTAGAAGGGCGTAGAGTG-3'
SEQ ID NO: 46
5'-TGTGTAGAAGGGTGTAGAGCG-3'
SEQ ID NO: 47
5'-TGTGTAGAAGGGCGTAGAGTG-3'
SEQ ID NO: 48
5'-TGCGTAGAAGGGTGTAGAGTG-3'
SEQ ID NO: 49
5'-CGTGTAGAAGGGCGTAGAGTG-3'
SEQ ID NO: 50
5'-CGTGTAGAAGGGTGTAGAGCG-3'

or substantially similar sequences.

In yet still another embodiment said gene is BCAT1 and:

(i) said first set of primers comprise the sequences:

SEQ ID NO: 97
(FWD PRIMER): chr12 (+); 24, 949, 138-24, 949, 164
5'-TTAGTGTTTTTTTGTTGATGTAATTCG-3'
SEQ ID NO: 65
(REV PRIMER): chr12 (-); 24, 949, 058-24, 949, 074
5'-CAATACCCGAAACGACGACG-3'

or substantially similar sequences and the probes directed to the amplification product of said first set of primers include the sequence:

SEQ ID NO: 66
5'-TTCGTCGCGAGAGGGTCGGTT-3'

or substantially similar sequences; and

(ii) said second set of primers comprise the sequences:

(FWD PRIMER): chr12 (-); 24, 949, 058-24, 949, 082
SEQ ID NO: 96
5'-TAGTGTTCGAGGCGGCGGCGAGTAT-3'
(REV PRIMER): chr12 (+); 24, 949, 140-24, 949, 159
SEQ ID NO: 62
5'-ATCTTCCTACTAATACAATCCGCTAAATC-3'

or substantially similar sequences and the probes directed to the amplification product of said second set of primers include the sequence:

SEQ ID NO: 63
5'-GATCGGTTTTTTCGCGGCGGA-3'

or substantially similar sequence.

In still yet another embodiment said gene is BCAT1 and:

(i) said first set of primers comprise the sequences:

(FWD PRIMER): chr12 (+); 24, 949, 131-24, 949, 159
SEQ ID NO: 64
5'-GTTTTTTTGTTGATGTAATTCGTTAGGTC-3'
(REV PRIMER): chr12 (-); 24, 949, 058-24, 949, 074
SEQ ID NO: 65
5'-CAATACCCGAAACGACGACG-3'

or substantially similar sequences and the probes directed to the amplification product of said first set of primers include the sequence:

SEQ ID NO: 66
5′-TTCGTCGCGAGAGGGTCGGTT-3′

or substantially similar sequences; and

(ii) said second set of primers comprise the sequences:

(FWD PRIMER): chr12 (−); 24,949,058-24,949,085
SEQ ID NO: 61
5′-TAGTGTTCGAGGCGGCGGCGAGTATACG-3′
(REV PRIMER): chr12 (+); 24,949,140-24,949,159
SEQ ID NO: 62
5′-ATCTTCCTACTAATACAATCCGCTAAATC-3′

or substantially similar sequences and the probes directed to the amplification product of said second set of primers include the sequence:

SEQ ID NO: 63
5′-GATCGGTTTTTTCGCGGCGGA-3′

or substantially similar sequence.

In a further embodiment said gene is IRF4 and:

(i) said first set of primers comprise the sequences:

(FWD PRIMER): Chr6 (+): 391,546-391,564
SEQ ID NO: 108
5′-GGCGCGGGAATTTTATTTC-3′
(REV PRIMER): Chr6 (−): 391,605-391,622
SEQ ID NO: 109
5′-AAACCGACCGAACGAACG-3′

or substantially similar sequences and the probes directed to the amplification product of said first set of primers include the sequence:

SEQ ID NO: 110
5′-AAAACCGACGTAAAAACTAAAAACTACCGCGA-3′

or substantially similar sequences; and

(ii) said second set of primers comprise the sequences:

(FWD PRIMER): Chr6 (−): 391,614-391,636
SEQ ID NO: 111
5′-TAAGTCGAGAGTCGGGGTCGGTC-3′
(REV PRIMER): Chr6 (+): 391,524-391,539
SEQ ID NO: 112
5′-CGACGCGCGAAAAATC-3′

or substantially similar sequences and the probes directed to the amplification product of said second set of primers include one or more of the sequences selected from:

SEQ ID NO: 113
5′-CCAACCTTCACGCCGACCCTAAAACTCG-3′
SEQ ID NO: 115
5′-CCTTCACGCCGACCCTAAAACTCG-3′

or substantially similar sequence.

In another further embodiment said gene is IRF4 and:

(i) said first set of primers comprise the sequences:

(FWD PRIMER): Chr6 (+): 391,546-391,564
SEQ ID NO: 108
5′-GGCGCGGGAATTTTATTTC-3′
(REV PRIMER): Chr6 (−): 391,605-391,622
SEQ ID NO: 109
5′-AAACCGACCGAACGAACG-3′

or substantially similar sequences and the probes directed to the amplification product of said first set of primers include the sequence:

SEQ ID NO: 110
5′-AAAACCGACGTAAAAACTAAAAACTACCGCGA-3′

or substantially similar sequences; and

(ii) said second set of primers comprise the sequences:

(FWD PRIMER): Chr6 (−): 391,614-391,630
SEQ ID NO: 114
5′-GAGAGTCGGGGTCGGTC-3′
(REV PRIMER): Chr6 (+): 391,524-391,539
SEQ ID NO: 112
5′-CGACGCGCGAAAAATC-3′

or substantially similar sequences and the probes directed to the amplification product of said second set of primers include one or more of the sequences selected from:

SEQ ID NO: 113
5′-CCAACCTTCACGCCGACCCTAAAACTCG-3′
SEQ ID NO: 115
5′-CCTTCACGCCGACCCTAAAACTCG-3′

or substantially similar sequence.

In yet another further embodiment said gene is IRF4 and:

(i) said first set of primers comprise the sequences:

(FWD PRIMER): Chr6 (+): 391,546-391,564
SEQ ID NO: 108
5′-GGCGCGGGAATTTTATTTC-3′
(REV PRIMER): Chr6 (−): 391,605-391,622
SEQ ID NO: 109
5′-AAACCGACCGAACGAACG-3″

or substantially similar sequences and the probes directed to the amplification product of said first set of primers include the sequence:

SEQ ID NO: 110
5′-AAAACCGACGTAAAAACTAAAAACTACCGCGA-3′

or substantially similar sequences; and

(ii) said second set of primers comprise the sequences:

(FWD PRIMER): Chr6 (−): 391,624-391,649
SEQ ID NO: 116
5′-GAGAGGGATTTTGTAAGTCGAGAGTC-3′
(REV PRIMER): Chr6 (+): 391,524-391,539
SEQ ID NO: 112
5′-CGACGCGCGAAAAATC-3′

or substantially similar sequences and the probes directed to the amplification product of said second set of primers include one or more of the sequences selected from:

SEQ ID NO: 113
5′-CCAACCTTCACGCCGACCCTAAAACTCG-3′
SEQ ID NO: 115
5′-CCTTCACGCCGACCCTAAAACTCG-3′

or substantially similar sequence.

The present invention is further described by reference to the following non-limiting examples.

### Example 1

### The Adverse Effect of Bilsulfite Treatment in Respect of PCR Amplifiable DNA

In order to determine how much of an effect bisulphite treatment has on the PCR amplification, the quantitation of amplicon, before and after bisulphite treatment, was compared using droplet digital PCR (ddPCR).

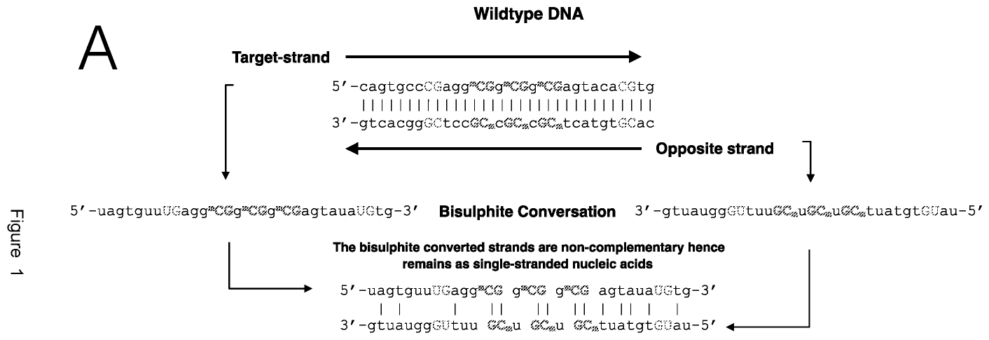

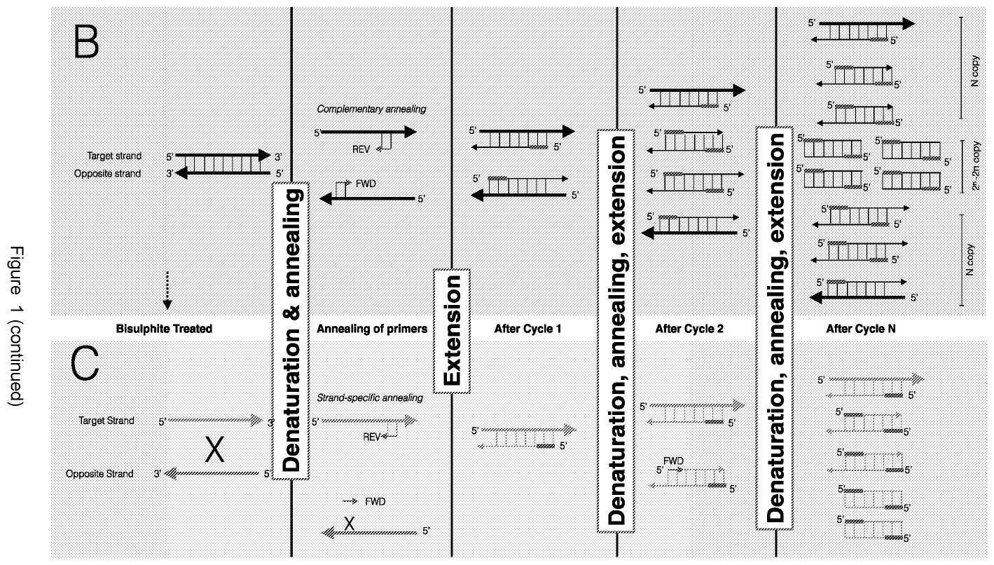

The concentration of fully-methylated human genome DNA (Millipore) was estimated by measuring the absorbance at 260 nm as per standard practice. The expected equivalent number of DNA copies were calculated and a portion of the DNA was treated with sodium bisulphite. The actual amount of PCR-amplifiable DNA copies, before and after bisulphite treatment, were determined by ddPCR using ACTB assays. The ddPCR ACTB assay used to estimate the DNA copies before bisulphite treatment detects and amplifies both of the complementary strands in the targeted region [SEQ IDs 67-71], whereas the assay to estimate DNA copies after bisulphite treatment detects and amplifies only one strand of DNA [SEQ IDs 72-76] as depicted in FIG. 1. Therefore, one would expect a quantitation of twice as many copies in wildtype DNA compare to bisulphite treated DNA, respectively. However, the observed loss was actually 10-40% higher than expected (Table 1).

In addition, target strand-specific assays for BCAT1 [SEQ IDs 57-58 & 64-66] or IKZF1 [SEQ IDs 3, 4, 11-13] on bisulphite converted DNA as template input were evaluated to determine if DNA loss and/or degradation is sequence-specific. Table 1 shows that in general BCAT1 and IKZF1 are quantified in similar amounts, but that these are amplified less abundantly than ACTB in bisulphite treated DNA. This indicates that DNA nicking/degradation may be sequence-specific and may be reflective of the fact that the amplicon regions targeted in BCAT1 and IKZF1 are methylated, whereas the ACTB amplicon region targeted is not from an area that is known to be methylated.

TABLE 1
Determination of amount of amplifiable DNA before and after bisulphite treatment.

| Estimated DNA copy | PCR amplifiable DNA—three marker regions | | | | | | |
|---|---|---|---|---|---|---|---|
| input/PCR reaction | (copies/reaction) | | | | | | |
| based on A₂₆₀nm | Before bisulphite treatment | | | After bisulphite treatment | | | % Loss |
| measurements | ACTB | BCAT1 | IKZF1 | ACTB | BCAT1 | IKZF1 | ACTB |
| 606 | 450 | n/a | n/a | 186.1 | 145.2 | 126.6 | 59 |
| 242 | 200 | n/a | n/a | 73.7 | 50.6 | 45.6 | 63 |
| 97 | 95 | n/a | n/a | 32.0 | 20.7 | 16.7 | 66 |
| 39 | 32 | n/a | n/a | 9.2 | 9.1 | 5.2 | 71 |
| 16 | 17 | n/a | n/a | 4.7 | 2.5 | 3.9 | 72 |
| 6 | 9.3 | n/a | n/a | 1.6 | 1.5 | 2.2 | 83 |
| 2.5 | 3.8 | n/a | n/a | 1.4 | 0.6 | 0.7 | 63 |
| 1 | 3 | n/a | n/a | 0.3 | 0 | 0 | 90 |
| 0 | 0 | n/a | n/a | 0 | 0 | 0 | — |
| DNA region SEQ ID | [67&68] | n/a | n/a | [72&73] | [57&58] | [3&4] | |
| Oligonucleotides [SEQ ID] | [69-71] | n/a | n/a | [74-76] | [64-66] | [11-21] | |

### Example 2

### Targeting Both Strands of Bisuphite Converted DNA

Fully methylated human genome DNA (2000 pg/well; Millipore) was used as the template and bisulphite converted using a customised Epitect Fast DNA Bisulphite kit (Qiagen) on a QIAcube HT (Qiagen). qPCR assays were designed targeting regions on either one or both strands of BCAT1 [SEQ IDs 64-66 and 61-63] and IKZF1 [SEQ IDs 11-21 and 22-32]. PCR reactions comprised 15 μL 2× Quantitect mastermix, 200 nM each Forward and Reverse primers and 100 nM each probe made up to 3 μL with nuclease-free water and 12 μL template DNA (166.7 pg/μL) and were cycled as follows: 95° C., 15 mins; [95° C., 15 secs; 62° C., 40 secs]×50; 40° C., 10 secs on a Light Cycler 480 (Roche).

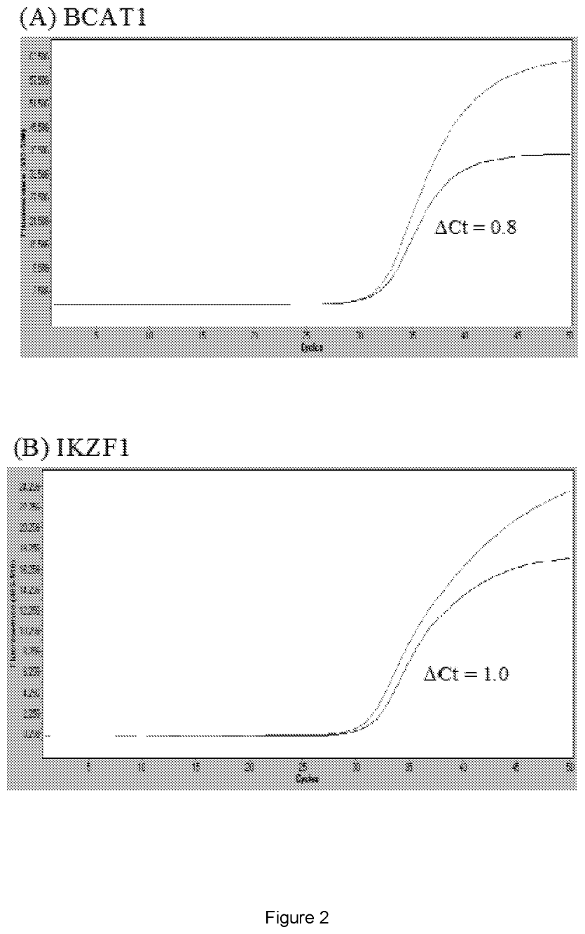

FIG. 2 shows the amplification results for A) BCAT1 and B) IKZF1. It can be seen that there is approximately double the amount of amplicon generated for both BCAT1 and IKZF1 when using primers that target both strands of DNA relative to using primers that target a single strand, thus confirming that the technology works as expected.

### Example 3

### ddPCR Quantification of Single or Double Stranded Amplification of Bisulphite Converted DNA

Droplet digital PCR (ddPCR) provides absolute quantification by distributing the DNA into approximately 20,000 discrete droplets, which then act as individual reaction vesicles for end-point PCR amplification. Following PCR amplification, the droplets are then assessed for fluorescence as an indicator that that particular droplet had at least one molecule of DNA in it to begin with. The number of fluorescent-positive droplets are counted and the number of copies of DNA in the original sample are calculated using the Poisson distribution to model the likelihood that each droplet contained one or more DNA molecules. Because the PCR amplification is end-point, the efficiency of the reaction is therefore unimportant, and the quantitation afforded by this technology is a more accurate determination.

To confirm whether the observed phenomenon in Example 2 was independent of PCR amplification efficiency the experiment above was repeated using ddPCR. Fully methylated human genome DNA (2000 pg/well; Millipore) was used as the template and bisulphite converted using a customised Epitect Fast DNA Bisulphite kit (Qiagen) on a QIAcube HT (Qiagen). ddPCR assays were designed targeting regions on target strand for ACTB [SEQ IDs 74-76] and target strand or both target strand and opposite strand for BCAT1 [SEQ IDs 64-66 and 61-63] and IKZF1 [SEQ IDs 11-21 and 22-32]. PCR reactions comprised 10 μL 2×ddPCR Supermix for Probes (no dUTP; BioRad), 450 nM each Forward and Reverse primers and 50-200 nM each probe made up to 2 μL with nuclease-free water and 8 μL template DNA (250 pg/μL). IKZF1 amplicons were detected using FAM labelled probes, ACTB was detected using a HEX probe and the target strand of BCAT1 was detected with a FAM probe and the target-opposite strand was detected with a HEX probe. Droplets were generated using a QX200 manual droplet generator (BioRad) and were subsequently cycled as follows: 95° C., 10 mins; [94° C., 30 secs; 58° C., 1 min]×45; 98° C., 10 mins on a C1000 (BioRad). Droplets were read on a QX200 droplet reader (BioRad) and the results were analysed using the Quantasoft Analysis Pro software (BioRad) and the number of positive droplets compared.

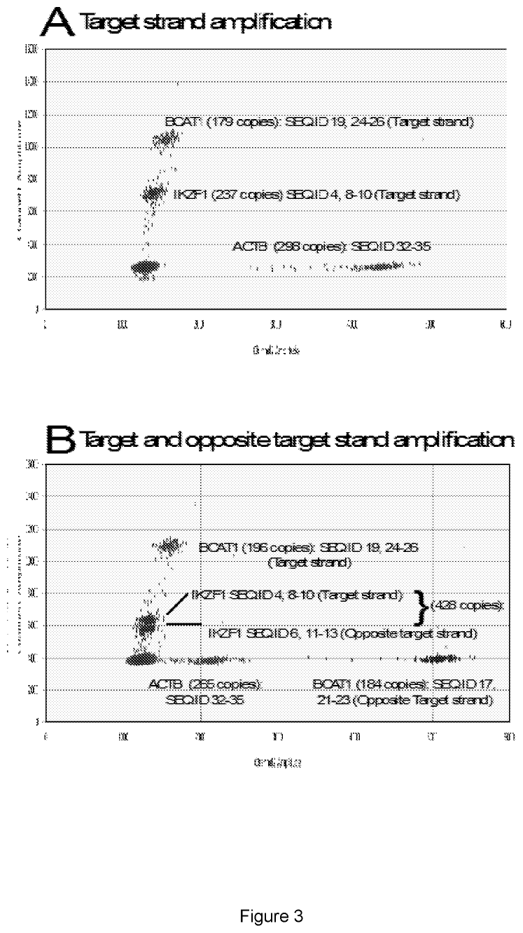

FIG. 3 shows the quantification results for A) target strand-specific amplification and B) double stranded amplification. Fluorescent populations are shown for the target-strand of BCAT1, IKZF1 and ACTB for both assays, and additional populations are observed for the BCAT1 and IKZF1 amplicons on the opposite strand in the assays simultaneously detecting both target- and opposite strands. The values given in brackets refer to the number of copies of each amplified gene per well, as determined by the Quantasoft software, and it can be seen that for BCAT1 and IKZF1 there is approximately twice the number of copies amplified in the target- and opposite strand assays relative to a target-specific strand assay only (179 v 380 copies/well, 212% and 237 v 428 copies/well, 180%, respectively). ACTB was only amplified on the target-specific strand in both assays and is present in roughly equal amounts in the two PCRs, 298 v 265 copies/well. The bisulphite treatment resulted in a 30-57% loss in PCR amplifiable material, which is in keeping with previous results, but does demonstrate the random nature of the nicking during bisulphite treatment.

These results confirm that targeting both strands of the non-complementary DNA following bisulphite conversion does unexpectedly nevertheless result in twice the amount of DNA being amplified, despite the significant loss of starting template due to bisulphite induced degradation and nicking.

### Example 4

### Improved Sensitivity of Methylation Specific PCR at Low DNA Input

The sensitivity of detection when DNA input amounts are very low, such as is the case when looking for methylated circulating tumour DNA in small quantities of plasma, is a significant problem in the art. In order to test this, samples were spiked with very low concentrations of methylated DNA in a background of excess unmethylated DNA and a comparison was made of the detection in assays where target strand or target- and opposite strands were detected.

Fully methylated human genome DNA (Millipore) and white blood cell unmethylated DNA (Roche) were bisulphite converted using a customised Epitect Fast DNA Bisulphite kit (Qiagen) on a QIAcube HT (Qiagen) and mixed to a ratio of 1:1667. Forty-five samples of this DNA mixture were analysed as triplicates in qPCR assays designed to target regions on the target-strand of ACTB [SEQ IDs 74-76] and one or both strands of BCAT1 [SEQ IDs 64-66 and 61-63] and IKZF1 [SEQ IDs 11-21 and 22-32]. ACTB amplification was used as a control to ensure that the PCR worked. PCR reactions comprised 15 μL 2× Quantitect mastermix, 200 nM each Forward and Reverse primers and 100 nM each probe made up to 3 μL with nuclease-free water and 12 μL template DNA containing 3 pg methylated DNA and 5 ng unmethylated DNA per well (equivalent to 1 copy of methylated DNA and 1515 copies of unmethylated DNA). Control samples contained 5 ng bisulphite converted, unmethylated DNA only. These were cycled as follows: 95° C., 15 mins; [95° C., 15 secs; 62° C., 40 secs]×50; 40° C., 10 secs on a Light Cycler 480 (Roche). A sample was deemed positive if one or more replicates were positive for either BCAT1 and/or IKZF1.

A total of 73% of samples were detected using the target-strand specific PCR, whereas 93% of samples were detected when both strands were targeted (Table 2). There was no assay positivity in samples containing 5 ng unmethylated DNA only. This increase in detection was statistically significant as determined by Fischer's Exact two-tailed test (p=0.02) and indicates that targeting both strands of non-complementary DNA results in much greater sensitivity in the ability to detect very low amounts of methylated DNA (˜1 copy per sample).

TABLE 2
Sample positivity in assays targeting single or both strands of
bisDNA on samples containing very low amounts of methylated DNA

| | Sample Methylation | |
|---|---|---|
| Assay configuration | Positivity (n = 45) | |
| Single strand | 33 (73%) | Fischer's Exact test, |
| Double strand | 42 (93%) | p value = 0.02 |

### Example 5

### Improved Detection in Plasma

Pooled plasma, from presumed negative Caucasian subjects under the age of 30 years, was spiked with fully methylated DNA (spiking range: 2.3-300 pg/mL, P0-P300) to simulate a real clinical sample. DNA was extracted from 4.5 mL samples (16 replicates per concentration) on a QIASymphony automated platform using a DSP virus/pathogen midi kit as per manufacturer's instruction (Qiagen). The eluted DNA was then bisulphite converted using a customised Epitect Fast DNA Bisulphite kit (Qiagen) on a QIAube HT (Qiagen). The resulting bisulphite converted DNA was analysed in triplicate (8 sample replicates per concentration) using qPCR assays designed to amplify the target-strand of ACTB [SEQ IDs 74-76] and one or both strands of BCAT1 [SEQ IDs 64-66 and 61-63] and IKZF1 [SEQ IDs 11-21 and 22-32]. ACTB amplification was used as a control to ensure that the extraction, bisulphite conversion and PCR worked. PCR reactions comprised 15 μL 2× Quantitect mastermix, 200 nM each Forward and Reverse primers and 100 nM each probe made up to 3 μL with nuclease-free water and 12 μL template DNA and were cycled as follows: 95° C., 15 mins; [95° C., 15 secs; 62° C., 40 secs]×50; 40° C., 10 secs, on a Light Cycler 480 (Roche). A sample was deemed positive if a replicate was positive for BCAT1 and/or IKZF1 on either DNA strand. The limit of detection (LOD) of the respective assays was calculated using Probit analysis.

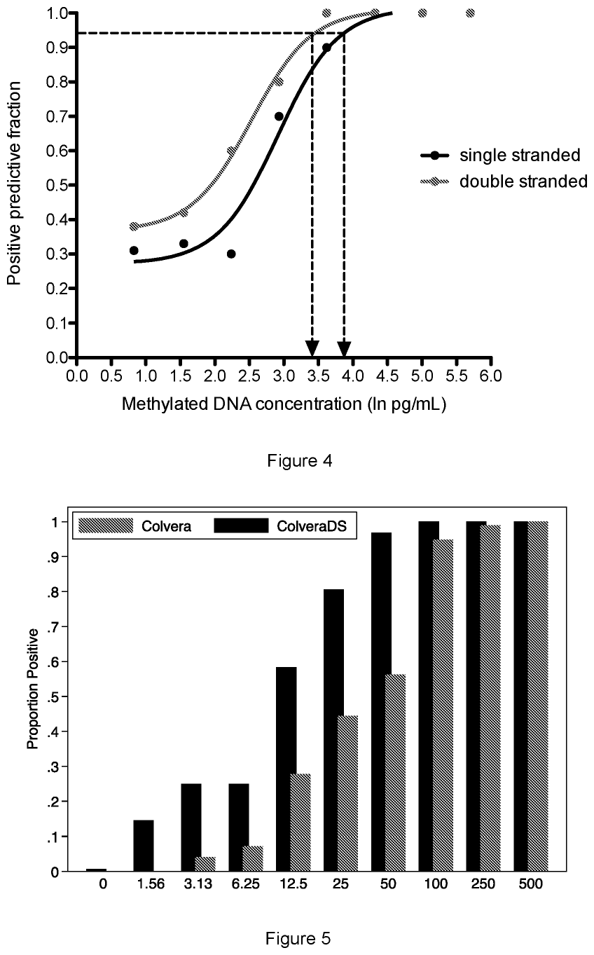

FIG. 4 is a graphical representation of the Probit analysis and shows that the LOD for the single-stranded PCR is 51.83 pg/mL plasma (˜15.7 copies/mL) and 26.98 pg/mL (˜8.2 copies/mL) for the PCR assay detected both the target specific strand and opposite strand. The LOD in a simulated clinical sample was almost half (52%) in the assay targeting both strands compared to the assay directed towards the target strand only, and again illustrates the utility of the invention in samples with low concentrations of methylated DNA.

### Example 6

### Improving Detection of Clinical Samples

Plasma collected from patients with colonoscopy-confirmed cases of colorectal cancer or no evidence of disease were tested to determine the clinical utility of the invention.

9 mL (2×4.5 mL) of plasma from 44 colonoscopy-confirmed CRC and 44 patients with no evidence of disease (NED) was extracted on a QIASymphony using a DSP virus/pathogen midi kit as per manufacturer's instruction (Qiagen). The eluted DNA was then bisulphite converted using a customised Epitect Fast DNA Bisulphite kit (Qiagen) on a QIAube HT (Qiagen). The resulting bisulphite converted DNA for each sample was split into 6 equal aliquots. The samples were analysed in triplicate using qPCR assays designed to amplify the target-strand of ACTB [SEQ IDs 74-76] and one or both strands of BCAT1 [SEQ IDs 64-66 and 61-63] and IKZF1 [SEQ IDs 11-21 and 22-32]. ACTB amplification was used as a control to ensure that the extraction, bisulphite conversion and PCR worked. PCR reactions comprised 15 μL 2× Quantitect mastermix, 200 nM each Forward and Reverse primers and 100 nM each probe made up to 3 μL with nuclease-free water and 12 μL template DNA and were cycled as follows: 95° C., 15 mins; [95° C., 15 secs; 62° C., 40 secs]×50; 40° C., 10 secs, on a Light Cycler 480 (Roche). A sample was deemed positive if a replicate was positive for BCAT1 and/or IKZF1 on either DNA strand.

Table 3 summarises the results, and any differences between the single-stranded and double-stranded assays with respect to overall assay positivity, as well as individual or total BCAT1 or IKZF1 replicate positivity, was assessed using a 2-tailed McNemar χ² test. The target-strand specific PCR (referred to below as a “single stranded assay”) detected 23/44 (52%) of cancer samples whereas the assay targeting both strands (referred to below as a “double stranded assay”) of bis DNA detected 30/44 (68%, p=0.07). In addition to the overall increase in assay positivity, there was a marked, statistically significant, increase in BCAT1 (p=0.0002), IKZF1 (p=0.0018) and combined (p=0.000) overall replicate positivity in the double-stranded assay compared to the single stranded assay. When looking at cancer stage, both BCAT1 and IKZF1 positivity in the double stranded assay is higher than in the single stranded assay, and statistically so at some stages. Of the 44 NED samples, the single stranded assay detected none, whereas 2/44 (4.5%) were detected by the double-stranded assay, both samples having a single BCAT1 replicate positive with late Cts of 40.48 and 43.97. This difference was not statistically significant.

These results confirm the clinical utility of the current method by demonstrating increased detection of cell free tumour DNA in plasma from patients with clinical disease, without a significant increase in non-specific detection from patients with no evidence of disease.

TABLE 3
Assay positivity and methylated BCAT and/or IKZF1 replicate positivity in DNA
extracted from plasma samples derived from patients that are colonoscopy-confirmed cancer patients
or with no evidence of disease, amplified using a single-stranded assay directed to the target strand or
an assay targeting both strands of the bis DNA strands.

| Single-stranded Assay | | | | | | Double-stranded Assay | | | | | |
|---|---|---|---|---|---|---|---|---|---|---|---|
| | | BCAT1 | IKZF1 | Total | | BCAT1 | | IKZF1 | | Total | |
| | Assay | reps | reps | replicate | Assay | reps | McNemar | reps | McNemar | replicate | McNemar |
| Stage | positive | positive | positive | positivity | positive | positive | χ² | positive | χ² | positivity | χ² |
| I | 2/3 | 2/9 | 4/9 | 6/18 | 3/3 | 7/9 | 0.025 | 4/9 | 1.000 | 11/18 | 0.182 |
| | (67%) | | | | (100%) | | | | | | |
| II | 9/14 | 15/42 | 14/42 | 29/84 | 10/14 | 23/42 | 0.021 | 14/42 | 1.000 | 37/84 | 0.061 |
| | (53%) | | | | (71%) | | | | | | |
| III | 8/15 | 17/45 | 17/45 | 34/90 | 11/15 | 23/45 | 0.034 | 26/45 | 0.003 | 49/90 | 0.0007 |
| | (53%) | | | | (67%) | | | | | | |
| Recurrent | 1/2 (50%) | 3/6 | 2/6 | 5/12 | 1/2 (50%) | 3/6 | 1.000 | 3/6 | 0.317 | 6/12 | 1.000 |
| Unknown | 3/10 | 6/30 | 1/30 | 7/60 | 5/10 | 8/30 | 0.414 | 6/30 | 0.059 | 14/60 | 0.096 |
| | (30%) | | | | (40%) | | | | | | |
| All | 23/44 | 43/132 | 38/132 | 81/264 | 30/44 | 64/132 | 0.0002 | 53/132 | 0.0018 | 117/264 | 0.000 |
| cancers | (52.3%) | (32.6%) | (28.8%) | (30.7%) | (68.2%)* | (48.5%) | | (40.2%) | | (44.3%) | |
| NED | 0/44 (0%) | 0/132 | 0/132 | 0/264 | 2/44 | 2/132 | 0.157 | 0/132 | 1.000 | 2/264 | 0.480 |
| | | | | (0%) | (4.5%) | | | | | (0.8%) | |

### Example 7

### Improved Detection in Plasma

Pooled plasma, from presumed healthy donors under the age of 30 years, was spiked with fully methylated DNA (spiking range: 0-500 pg/mL) to simulate a real clinical sample. DNA was extracted from 4.5 mL samples on a QIASymphony automated platform using either the QS DSP virus/pathogen midi or QS DSP circulating DNA kits (Qiagen). The eluted DNA was then bisulphite converted using a customised Epitect Fast DNA Bisulphite kit (Qiagen) on a QIAube HT (Qiagen). The resulting bisulphite converted DNA was analysed in triplicate (12-24 sample replicates per concentration) using qPCR assays designed to amplify the target-strand of ACTB [SEQ IDs 74-76] and one or both strands of BCAT1 [SEQ IDs 62-63, 96, 65-66, 97] and IKZF1 [SEQ IDs 11-21, 77-95]. ACTB amplification was used as a control to ensure that the extraction, bisulphite conversion and PCR worked [SEQ IDs 74-76]. PCR reactions comprised 15 μL 2× Quantitect mastermix, 200 nM each Forward and Reverse primers and 100 nM each probe made up to 3 μL with nuclease-free water and 12 μL template DNA and were cycled as follows: 95° C., 15 mins; [95° C., 15 secs; 62° C., 40 secs]×50; 40° C., 10 secs, on a Light Cycler 480 (Roche). The relationship between methylated DNA concentration and test positivity was assessed by probit regression modelling, and the LOD was estimated for each method as the concentration that resulted in 95% probability of determining a positive result. Samples were tested via real time PCR in triplicate and a sample was deemed positive if any of the 3 replicates for either methylated BCAT1 or IKZF1 was detected (6 replicates in total). A 2-fold improvement in limit of detection was observed when simultaneously detecting both target regions and opposite target regions as opposed to only detecting targeted regions (10.7 pg/mL vs 21.4 pg/mL). The improved sensitivity was also reflected in an increase in PCR replicate positivity. Refer to Table 4 and FIG. 5.

TABLE 4
Comparison of replicate and sample positivity between Single
and Double strand assays

| Methylated | | | Single Target | | Targeting Regions | |
|---|---|---|---|---|---|---|
| DNA | N | | Regions, Pos (%) | | of interest on both | |
| Conc. | Sam- | Rep- | Sam- | Rep- | strands, Pos (%) | |
| (pg/mL) | ple | licate | ple | licate | Sample | Replicate |
| 0 | 24 | 144 | 0 (0) | 0 (0) | 1 (4.2) | 1 (0.7) |
| 1.56 | 16 | 96 | 0 (0) | 0 (0) | 9 (56.3) | 14 (14.6) |
| 3.13 | 12 | 72 | 3 (25) | 3 (4.2) | 9 (75) | 18 (25) |
| 6.25 | 16 | 96 | 6 (37.5) | 7 (7.3) | 12 (75) | 24 (25) |
| 12.5 | 12 | 72 | 9 (75) | 20 (27.8) | 11 (91.7) | 42 (58.3) |
| 25 | 12 | 72 | 11 (91.7) | 32 (44.4) | 12 (100) | 58 (80.6) |
| 50 | 16 | 96 | 16 (100) | 54 (56.3) | 16 (100) | 93 (96.9) |
| 100 | 16 | 96 | 16 (100) | 91 (94.8) | 16 (100) | 96 (100) |
| 250 | 16 | 96 | 16 (100) | 95 (99) | 16 (100) | 96 (100) |
| 500 | 12 | 72 | 12 (100) | 72 (100) | 12 (100) | 72 (100) |

### Example 8

### Improved Sensitivity of Methylation Specific PCR at Low DNA Input

Universal methylated Human Genome DNA (Zymo, cat # D5011) was fragmented via sonication to reflect circulating cell free DNA (ccfDNA) found in plasma (˜100-500 bp fragments). Fragmented DNA was bisulphite converted, quantified using ddPCR [SEQ IDs 11-21, 64-66, 74-76] and diluted to −1 genomic copy of methylated DNA in a background of 1250 copies bisulphite converted unmethylated DNA per sample (45 μL). The 45 samples were analysed as triplicates in qPCR assays designed to target regions on the target-strand of ACTB [SEQ IDs 74-76] and one or both strands of BCAT1 [SEQ IDs 62-63, 96, 65-66, 97] and IKZF1 [SEQ IDs 11-21, 77-95].

PCR reactions comprised 15 μL 2× Quantitect mastermix, 200 nM each Forward and Reverse primers and 100 nM each probe made up to 3 μL with nuclease-free water and 12 μL template DNA. Control samples contained 1250 copies of bisulphite converted, unmethylated DNA only. These were cycled as follows: 95° C., 15 mins; [95° C., 15 secs; 62° C., 40 secs]×50; 40° C., 10 secs on a Light Cycler 480 (Roche). A sample was deemed positive if one or more replicates were positive for either BCAT1 and/or IKZF1.

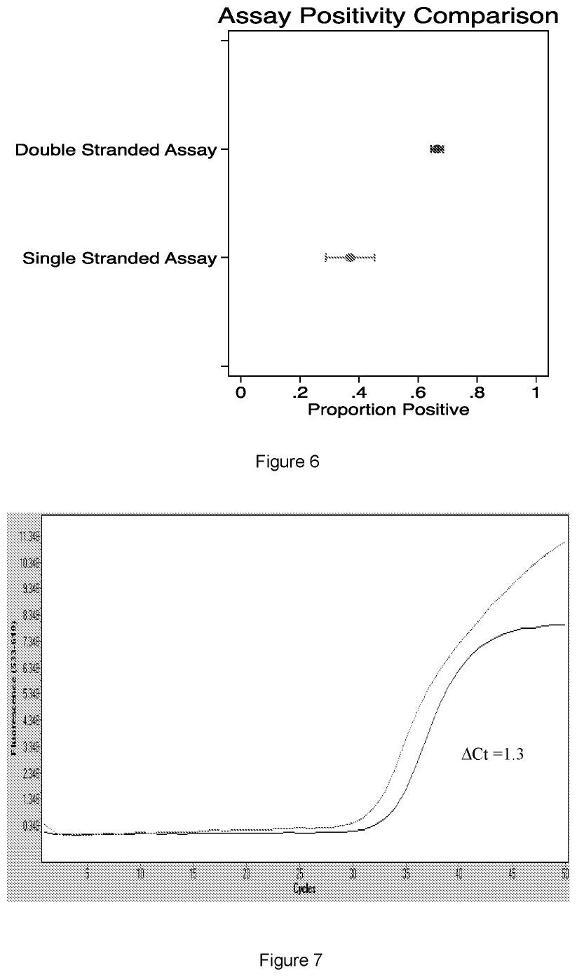

A total of 37% of samples were detected using the target-strand specific PCR, whereas 67% of samples were detected when both strands were targeted (p-value=<0.001 Fischer's Exact two-tailed test; FIG. 6). Multiple runs were performed with the assay targeting both strands of BCAT1 and IKZF1 using different lots of PCR Mastermix and oligonucleotide mix generating consistently improved positivity for said assay compared to an assay targeted only one strand of BCAT1 and IKZF1, FIG. 6. This indicates that targeting both strands of non-complementary DNA results in much greater sensitivity in the ability to detect very low amounts of methylated DNA (˜1 copy per sample).

### Example 9

### Clinical Demonstration of Improved Sensitivity

Plasma collected from patients with colonoscopy-confirmed cases of colorectal cancer or no evidence of disease were tested to determine the clinical utility of the invention (2×4.5 mL). The study cohort included 1,576 subjects, comprised of 1,536 colonoscopy confirmed subjects and 40 presumed healthy donors (<30 years of age). Circulating DNA was isolated, bisulphite converted and PCR assayed as described in Example 7.

Table 5 summarises the results, and any differences between the single-stranded and double-stranded assays with respect to overall assay positivity, as well as individual or total BCAT1 or IKZF1 replicate positivity, was assessed using a 2-tailed McNemar χ² test. The target-strand specific PCR (referred to below as a “single stranded assay”) detected 106/177 (59.9%) of cancer samples whereas the assay targeting both strands (referred to below as a “double stranded assay”) of bis DNA detected 127/177 (70.1%, p=0.0067). In addition to the overall increase in assay positivity, there was a marked increase in BCAT1, IKZF1 positivity in the double-stranded assay compared to the single stranded assay. When looking at cancer stage, both BCAT1 and IKZF1 positivity in the double stranded assay is higher than in the single stranded assay. Of the 1072 samples from subjects with no neoplasia, the single stranded assay detected 78 (7.3%), whereas 163 (15.2%) were detected by the double-stranded assay (p=0.0019)

These results confirm the clinical utility of the invention by demonstrating increased detection of cell free tumour DNA in plasma from patients with clinical disease.

TABLE 5
Summary of results of analysis of 1,576 individual clinical samples with the single-
and double-stranded assays.
Positivity Rates, No Positive. (%)

| | | Single stranded Assay | | | Double stranded Assay | | | |
|---|---|---|---|---|---|---|---|---|
| Phenotype | N | BCAT1 | IKZF1 | Either | BCAT1 | IKZF1 | Either | P-value¹ |
| Colon Cancer | 177 | 88 | 75 | 106 | 113 | 91 | 124 | 0.0067 |
| | | (49.7) | (42.4) | (59.9) | (63.8) | (51.4) | (70.1) | |
| Stage I | 43 | 13 | 11 | 17 (39.5) | 19 (44.2) | 13 | 20 (46.5) | |
| | | (30.2) | (25.6) | | | (30.2) | | |
| Stage II | 57 | 29 | 25 | 37 (64.9) | 42 (73.7) | 33 | 45 (78.9) | |
| | | (50.9) | (43.9) | | | (57.9) | | |
| Stage III | 53 | 29 | 26 | 34 (64.2) | 33 (62.3) | 29 | 39 (73.6) | |
| | | (54.7) | (49.1) | | | (54.7) | | |
| Stage IV | 18 | 14 | 12 | 15 (83.3) | 16 (88.9) | 14 | 16 (88.9) | |
| | | (77.8) | (66.7) | | | (77.8) | | |
| Unstaged | 6 | 3 (50) | 1 (16.7) | 3 (50) | 3 (50) | 2 (33.3) | 4 (66.7) | |
| Adenoma | 296 | 23 (7.8) | 11 (3.7) | 30 (10.1) | 48 (16.2) | 9 (3) | 53 (17.9) | <0.001 |
| Adv. Adenoma | 149 | 11 (7.4) | 3 (2) | 13 (8.7) | 26 (17.4) | 5 (3.4) | 30 (20.1) | |
| Non Adv. Adenoma | 147 | 12 (8.2) | 8 (5.4) | 17 (11.6) | 22 (15) | 4 (2.7) | 23 (15.6) | |
| No Neoplasia | 1072 | 60(5.6) | 27 (2.5) | 78 (7.3) | 147 | 31 (2.9) | 163 | 0.0019 |
| | | | | | (13.7) | | (15.2) | |
| Non-neoplastic | 618 | 36 (5.8) | 18 (2.9) | 48 (7.8) | 104 | 20 (3.2) | 113 | |
| | | | | | (16.8) | | (18.3) | |
| IBD | 62 | 5 (8.1) | 0 (0) | 5 (8.1) | 8 (12.9) | 0 (0) | 8 (12.9) | |
| No Evidence of | 352 | 19 (5.4) | 8 (2.3) | 24 (6.8) | 32 (9.1) | 11 (3.1) | 39 (11.1) | |
| Disease | | | | | | | | |
| Presumed healthy | 40 | 0 (0) | 1 (2.5) | 1 (2.5) | 3 (7.5) | 0 (0) | 3 (7.5) | |
| Other | 31 | 3 (9.7) | 0 (0) | 3 (9.7) | 6 (19.4) | 2 (6.5) | 6 (19.4) | 0.2568 |

¹McNemar's Chi Square Test

### Example 10

### Targeting Both Strands of Bisulphite Converted DNA

Fully methylated human genome DNA (2000 pg/well; Millipore) was used as the template and bisulphite converted using a customised Epitect Fast DNA Bisulphite kit (Qiagen) on a QIAcube HT (Qiagen). qPCR assays were designed targeting regions on either one or both strands of IRF4 [SEQ IDs 108-110 and 111-113]. PCR reactions comprised 15 μL 2× Quantitect mastermix, 200 nM each Forward and Reverse primers and 100 nM each probe made up to 3 μL with nuclease-free water and 12 μL template DNA (166.7 pg/μL) and were cycled as follows: 95° C., 15 mins; [95° C., 15 secs; 62° C., 40 secs]×50; 40° C., 10 secs on a Light Cycler 480 (Roche).

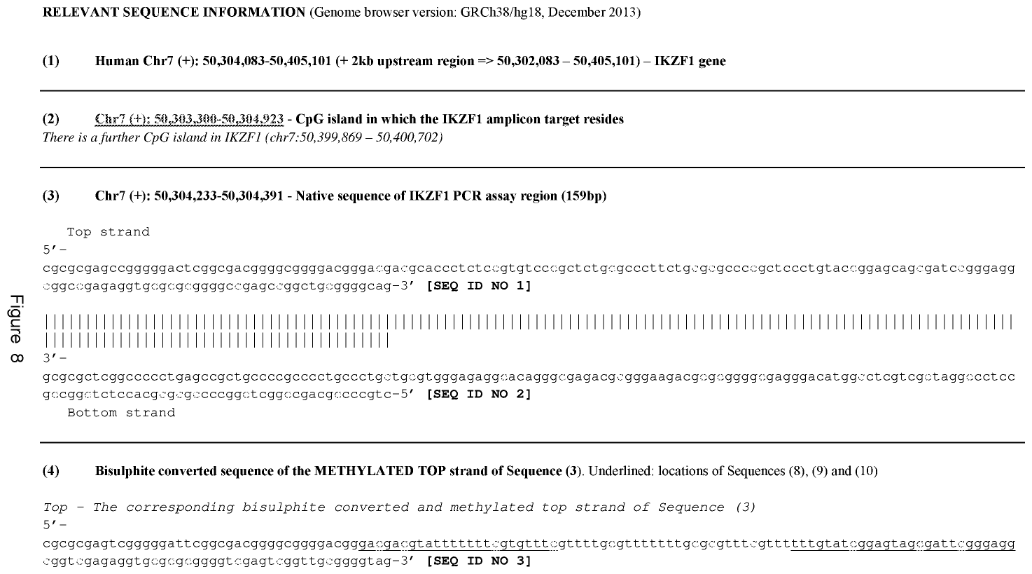

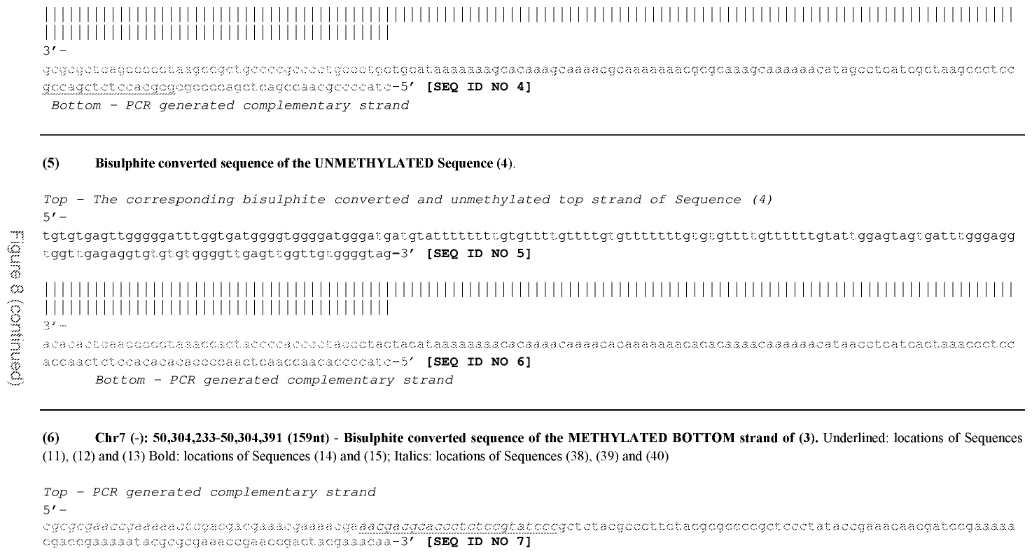

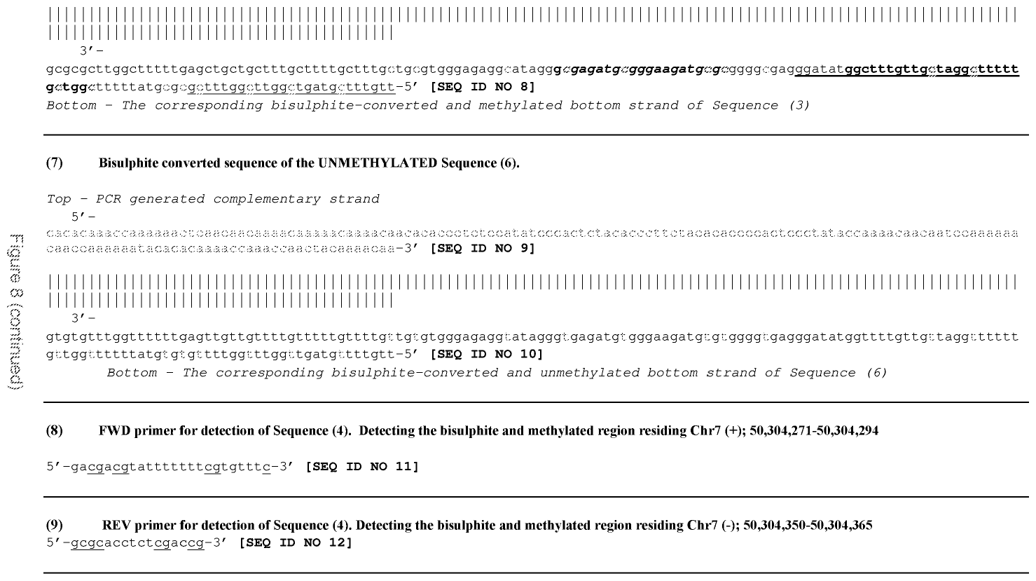

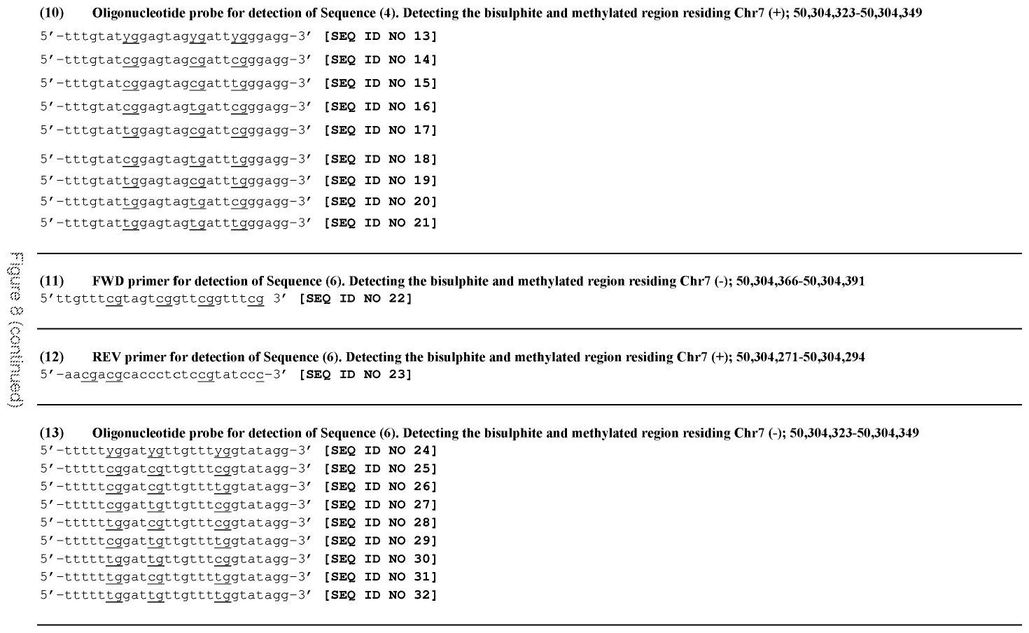

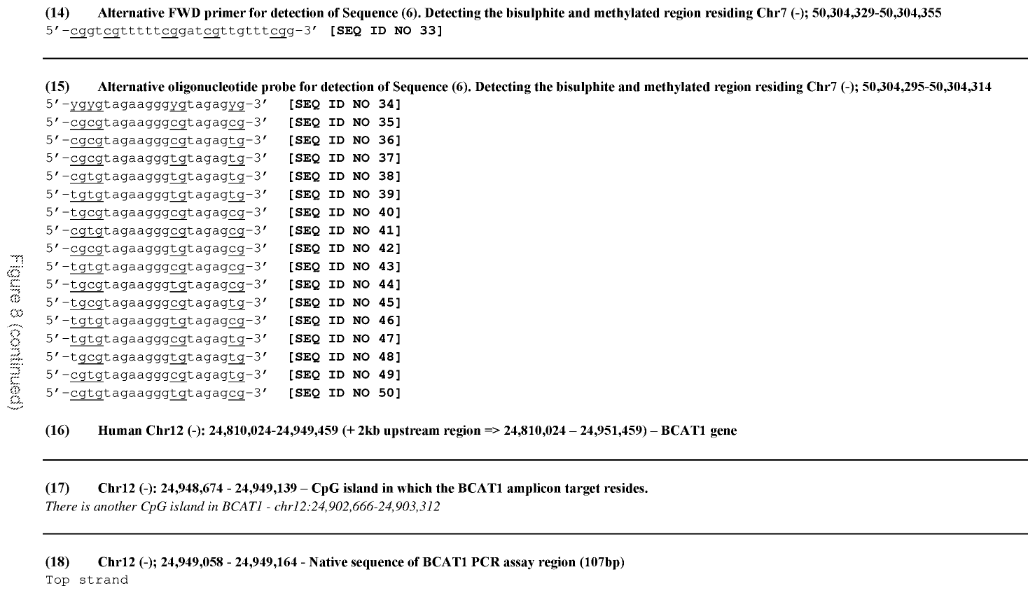

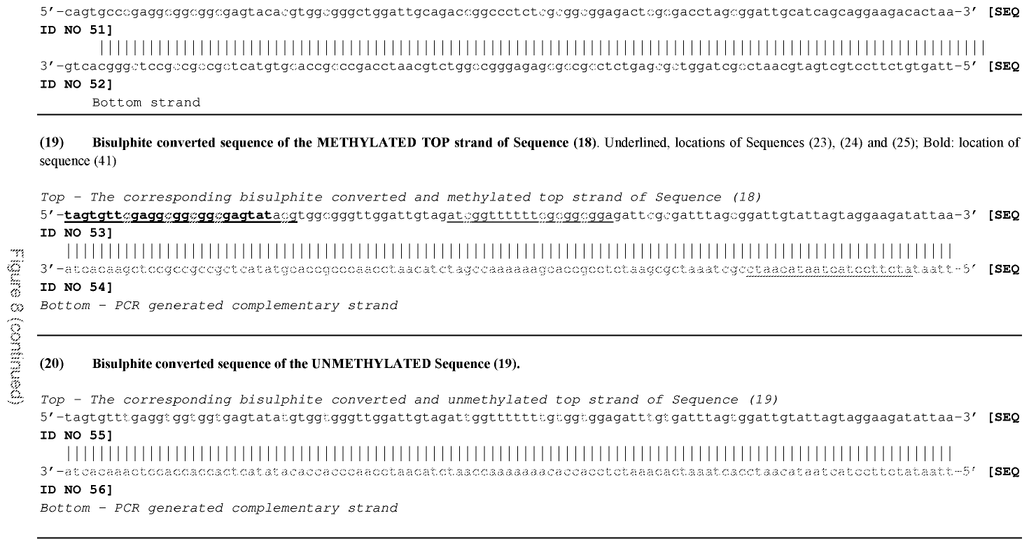

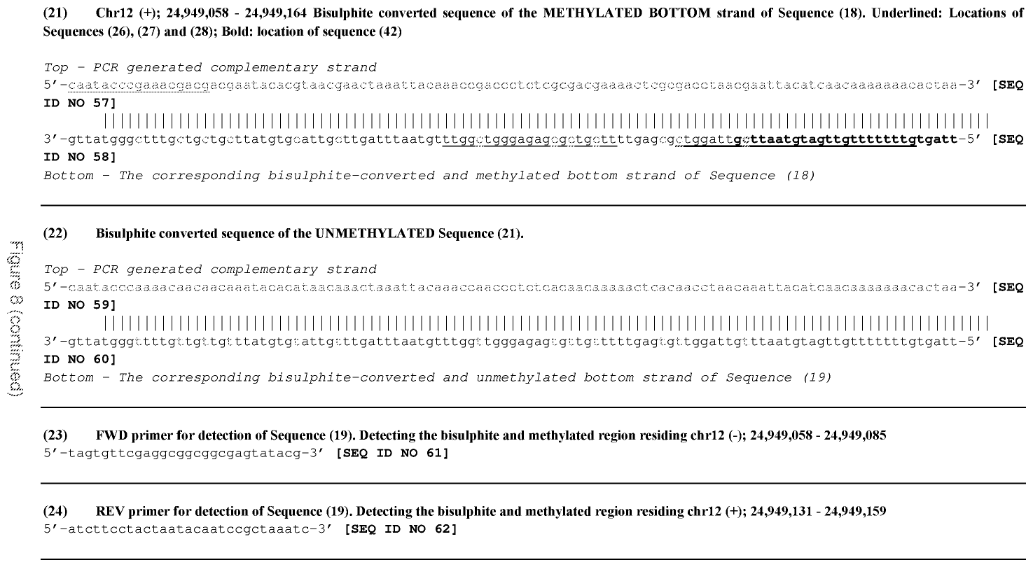

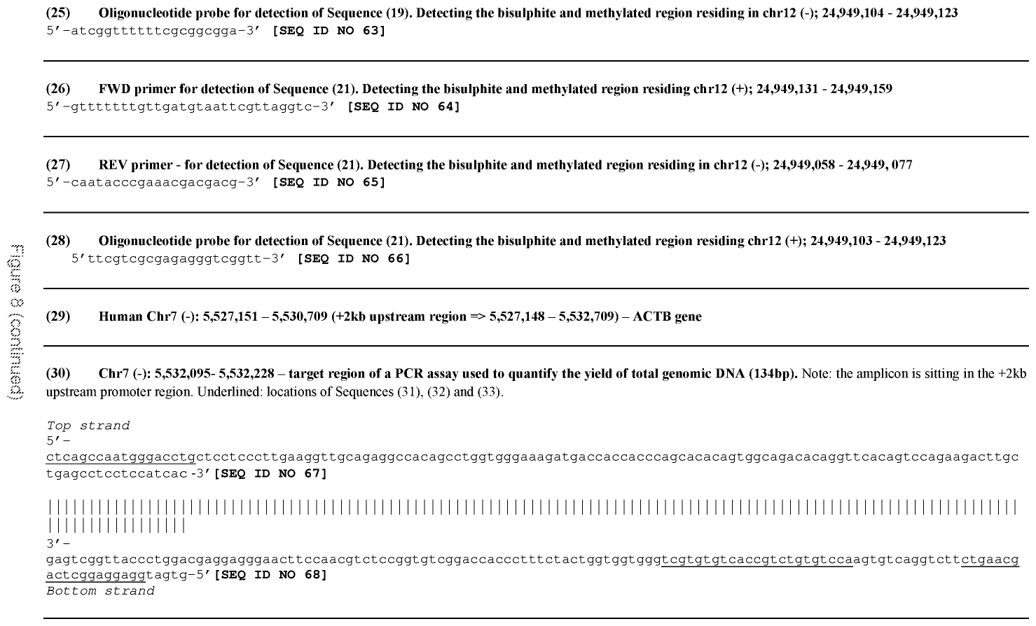

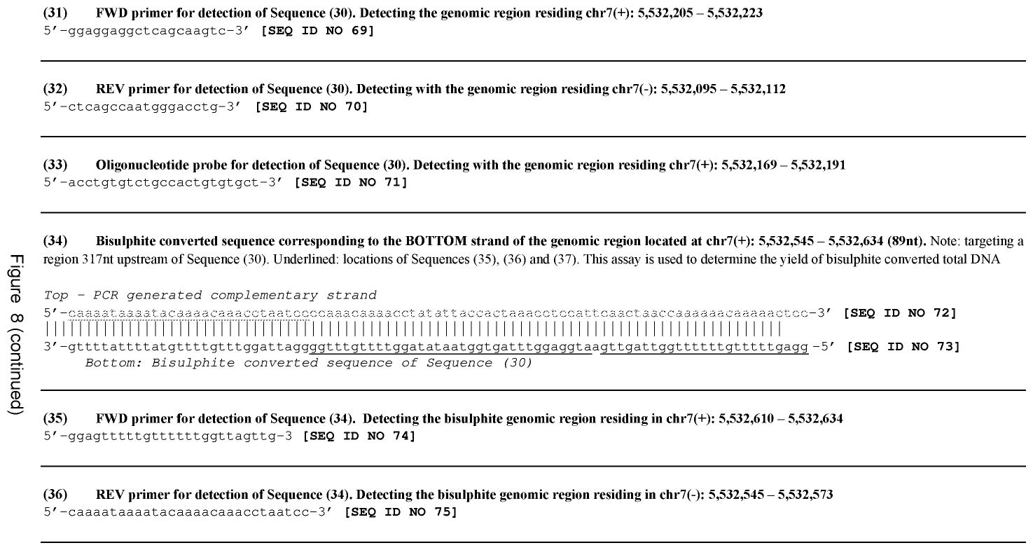

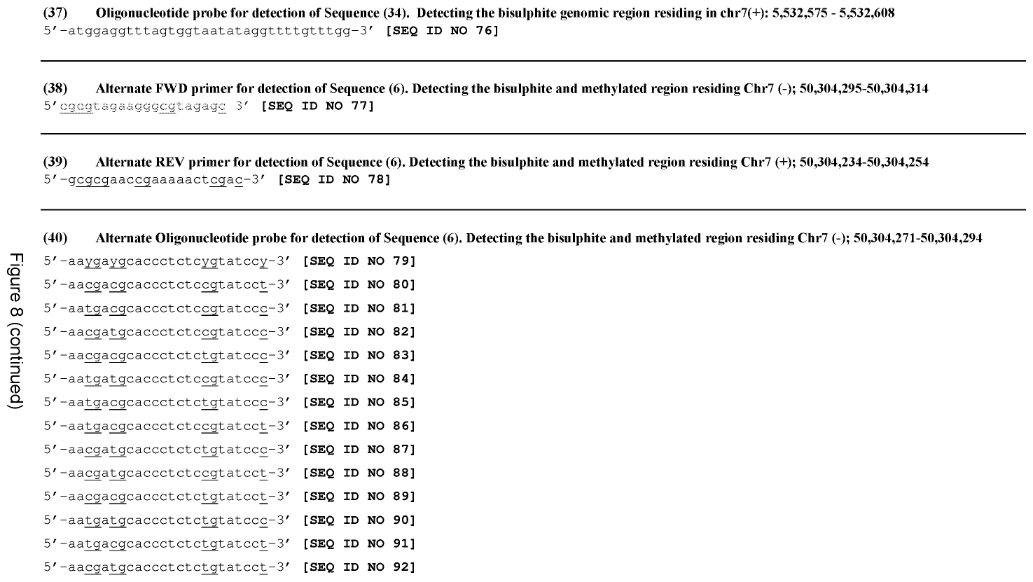

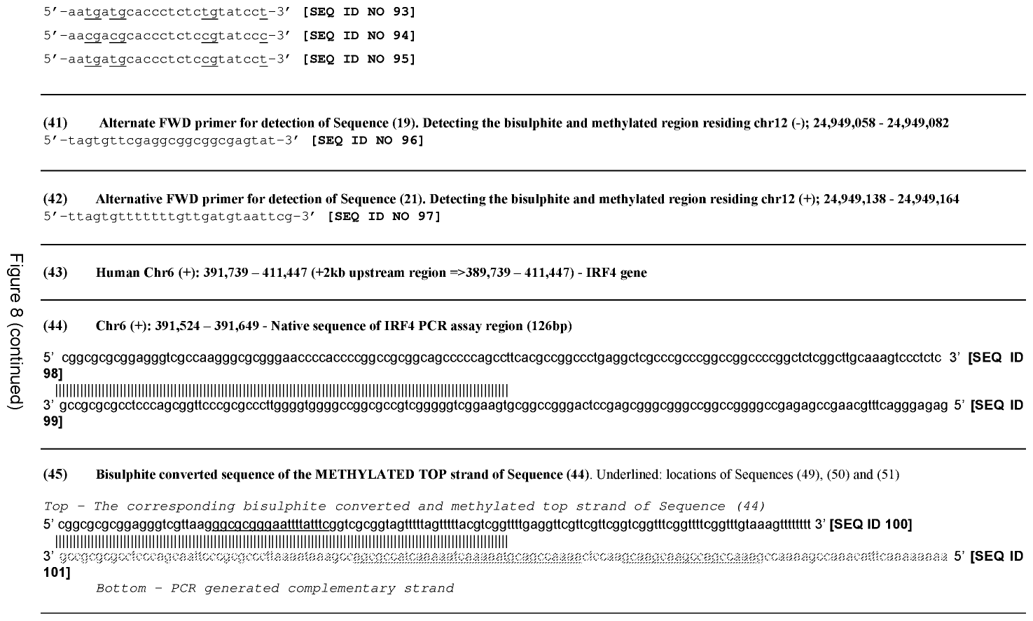

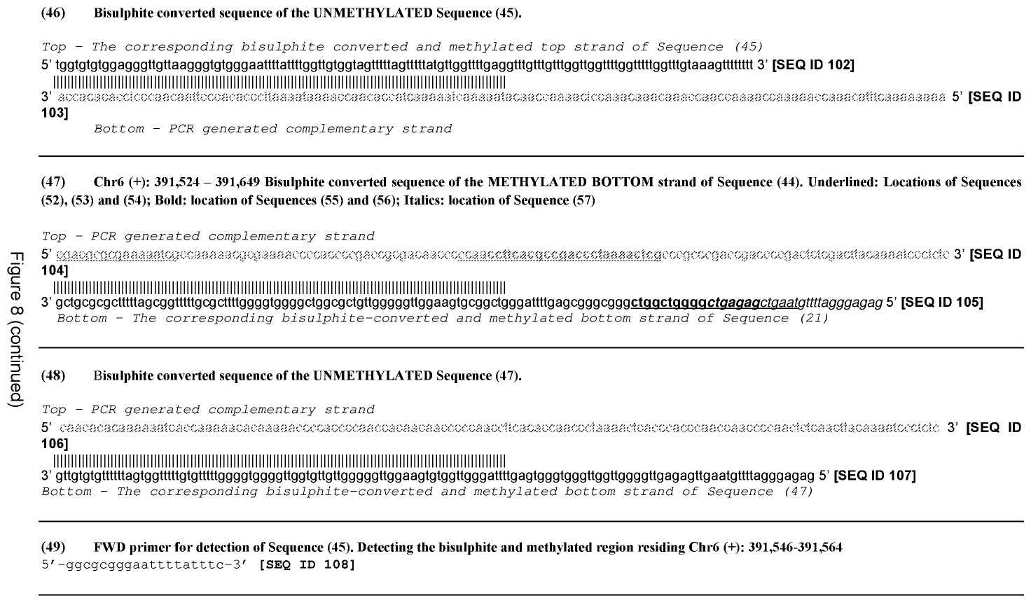

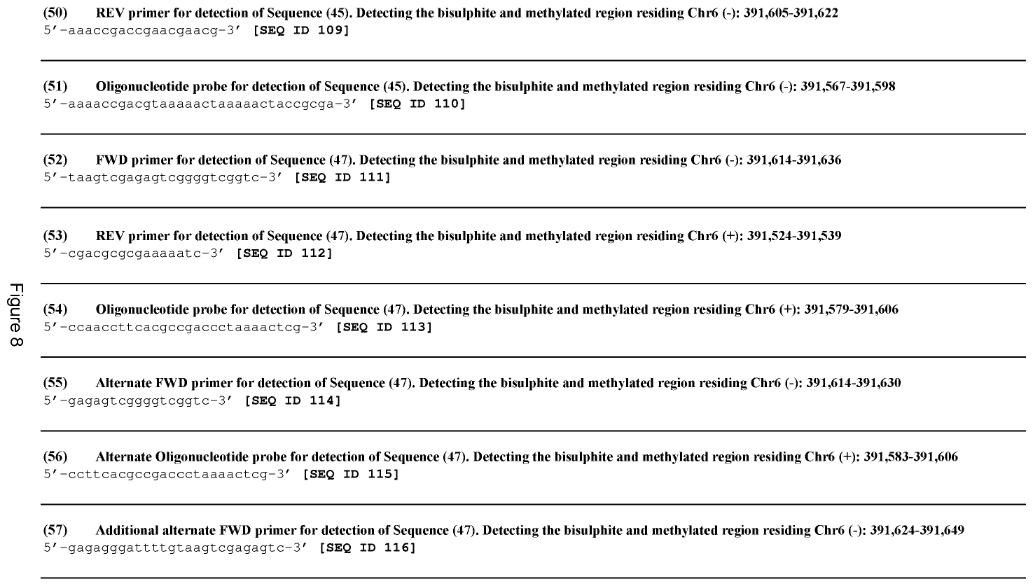

FIG. 7 shows the amplification results for IRF4. It can be seen that there is approximately double the amount of amplicon generated for IRF4 when using primers that target both strands of DNA relative to using primers that target a single strand, thus confirming that the technology works as expected on a different gene.

### Example 11

### Improved Sensitivity of Methylation Specific PCR at Low DNA Input

Universal methylated Human Genome DNA (Millipore, cat # S7821) was fragmented via sonication to reflect circulating cell free DNA (ccfDNA) found in plasma (˜100-500 bp fragments). Fragmented DNA was bisulphite converted, quantified using ddPCR [SEQ IDs 11-21, 64-66, 74-76] and diluted to −1 genomic copy of methylated DNA in a background of 1250 copies bisulphite converted unmethylated DNA (Millipore, cat #7822) per sample (454). The 45 samples were analysed as triplicates in qPCR assays designed to target regions on the target-strand of ACTB [SEQ IDs 74-76], IKZF1 [SEQ IDs 11-21] and IRF4 [SEQ IDs 108-110], and one or both strands of BCAT1 [SEQ IDs 62-63, 96, 65-66, 97].

PCR reactions comprised 15 μL 2× Quantitect mastermix, 200 nM each Forward and Reverse primers and 100 nM each probe made up to 3 μL with nuclease-free water and 12 μL template DNA. Control samples contained 1250 copies of bisulphite converted, unmethylated DNA only. These were cycled as follows: 95° C., 15 mins; [95° C., 15 secs; 62° C., 40 secs]×50; 40° C., 10 secs on a Light Cycler 480 (Roche). Both IKZF1 and IRF4 were detected on the FAM channel, whereas both strands of BCAT1 were detected on the HEX channel. A sample was deemed positive if one or more replicates were positive for either BCAT1, IKZF1 and/or IRF4.

A total of 23.7% (IKZF1) and 22.2% (BCAT1) of replicates and 77.8% of samples were detected using the single target-strand specific PCR, whereas 35.6% (single strand of IKZF1 plus IRF4) and 45.2% (double stranded BCAT1) of replicates and 93.3% of samples were detected when both strands were targeted (p-value=0.0169, 0.0001 and 0.069, respectively, Fischer's Exact two-tailed test; Table 6). This indicates that targeting both strands of non-complementary DNA, or adding in additional markers on the target strand, results in much greater sensitivity in the ability to detect very low amounts of methylated DNA (˜1 copy per sample).

TABLE 6
Replicate and sample positivity in single versus double stranded assay.

| | No | | No | | | |
|---|---|---|---|---|---|---|
| | replicates | Fischer's | replicates | Fischer's | No | Fischer's |
| | positive | exact | positive | exact | samples | exact |
| Assay | FAM | test | HEX | test | positive | test |
| Single | 32/135 | N/A | 30/135 | N/A | 35/45 | N/A |
| stranded | (23.7%) | | (22.2%) | | (77.8%) | |
| Double | 48/135 | 0.0169 | 61/135 | 0.0001 | 42/45 | 0.069 |
| stranded | (35.6%) | | (45.2%) | | (93.3%) | |

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

A. R. Thierry, F. Mouliere, S. El Messaoudi, C. Mollevi, E. Lopez-Crapez, F. Rolet, B. Gillet, C. Gongora, P. Dechelotte, B. Robert, M. Del Rio, P.-J. Lamy, F. Bibeau, M. Nouaille, V. Loriot, A.-S. Jarrousse, F. Molina, M. Mathonnet, D. Pezet, and M. Ychou, “Clinical validation of the detection of KRAS and BRAF mutations from circulating tumor DNA.,” Nature Med, vol. 20, no. 4, pp. 430-435, April 2014.
B. Vogelstein, N. Papadopoulos, V. E. Velculescu, S. Zhou, L. A. Diaz, and K. W. Kinzler, “Cancer genome landscapes.,” Science, vol. 339, no. 6127, pp. 1546-1558, March 2013.
C. Bettegowda, M. Sausen, R. J. Leary, I. Kinde, Y. Wang, N. Agrawal, B. R. Bartlett, H. Wang, B. Luber, R. M. Alani, E. S. Antonarakis, N. S. Azad, A. Bardelli, H. Brem, J. L. Cameron, C. C. Lee, L. A. Fecher, G. L. Gallia, P. Gibbs, D. Le, R. L. Giuntoli, M. Goggins, M. D. Hogarty, M. Holdhoff, S.-M. Hong, Y. Jiao, H. H. Juhl, J. J. Kim, G. Siravegna, D. A. Laheru, C. Lauricella, M. Lim, E. J. Lipson, S. K. N. Marie, G. J. Netto, K. S. Oliner, A. Olivi, L. Olsson, G. J. Riggins, A. Sartore-Bianchi, K. Schmidt, L.-M. Shih, S. M. Oba-Shinjo, S. Siena, D. Theodorescu, J. Tie, T. T. Harkins, S. Veronese, T.-L. Wang, J. D. Weingart, C. L. Wolfgang, L. D. Wood, D. Xing, R. H. Hruban, J. Wu, P. J. Allen, C. M. Schmidt, M. A. Choti, V. E. Velculescu, K. W. Kinzler, B. Vogelstein, N. Papadopoulos, and L. A. Diaz, “Detection of circulating tumor DNA in early- and late-stage human malignancies.,” Sci Transl Med, vol. 6, no. 224, pp. 224ra24-224ra24, February 2014.
F. Diehl, M. Li, D. Dressman, Y. He, D. Shen, S. Szabo, L. A. Diaz, S. N. Goodman, K. A. David, H. Juhl, K. W. Kinzler, and B. Vogelstein, “Detection and quantification of mutations in the plasma of patients with colorectal tumors.,” Proceedings of the National Academy of Sciences, vol. 102, no. 45, pp. 16368-16373, November 2005.
F. Mouliere, B. Robert, E. Arnau Peyrotte, M. Del Rio, M. Ychou, F. Molina, C. Gongora, and A. R. Thierry, “High fragmentation characterizes tumour-derived circulating DNA.,” PLoS ONE, vol. 6, no. 9, p. e23418, 2011.
H. Schwarzenbach, D. S. B. Hoon, and K. Pantel, “Cell-free nucleic acids as biomarkers in cancer patients.,” Nat Rev Cancer, vol. 11, no. 6, pp. 426-437, June 2011.
I. B. Roninson, E. V. Broude, and B. D. Chang, “If not apoptosis, then what? Treatment-induced senescence and mitotic catastrophe in tumor cells.,” Drug Resist. Updat., vol. 4, no. 5, pp. 303-313, October 2001.
K. C. A. Chan, J. Zhang, A. B. Y. Hui, N. Wong, T. K. Lau, T. N. Leung, K.-W. Lo, D. W. S. Huang, and Y. M. D. Lo, “Size distributions of maternal and fetal DNA in maternal plasma.,” Clin Chem, vol. 50, no. 1, pp. 88-92, January 2004.
K. Warton and G. Samimi, “Methylation of cell-free circulating DNA in the diagnosis of cancer.,” Front Mol Biosci, vol. 2, p. 13,2015.
M. S. Lawrence, P. Stojanov, P. Polak, G. V. Kryukov, K. Cibulskis, A. Sivachenko, S. L. Carter, C. Stewart, C. H. Mermel, S. A. Roberts, A. Kiezun, P. S. Hammerman, A. McKenna, Y. Drier, L. Zou, A. H. Ramos, T. J. Pugh, N. Stransky, E. Heiman, J. Kim, C. Sougnez, L. Ambrogio, E. Nickerson, E. Shefler, M. L. Cortes, D. Auclair, G. Saksena, D. Voet, M. Noble, D. DiCara, P. Lin, L. Lichtenstein, D. I. Heiman, T. Fennell, M. Imielinski, B. Hernandez, E. Hodis, S. Baca, A. M. Dulak, J. Lohr, D.-A. Landau, C. J. Wu, J. Melendez-Zajgla, A. Hidalgo-Miranda, A. Koren, S. A. McCarroll, J. Mora, R. S. Lee, B. Crompton, R. Onofrio, M. Parkin, W. Winckler, K. Ardlie, S. B. Gabriel, C. W. M. Roberts, J. A. Biegel, K. Stegmaier, A. J. Bass, L. A. Garraway, M. Meyerson, T. R. Golub, D. A. Gordenin, S. Sunyaev, E. S. Lander, and G. Getz, “Mutational heterogeneity in cancer and the search for new cancer-associated genes,” Nature, vol. 499, no. 7457, pp. 214-218, July 2013.
P. Bordi, M. Del Re, R. Danesi, and M. Tiseo, “Circulating DNA in diagnosis and monitoring EGFR gene mutations in advanced non-small cell lung cancer.,” Transl Lung Cancer Res, vol. 4, no. 5, pp. 584-597, October 2015.
P. O. Delgado, B. C. A. Alves, F. de S. Gehrke, R. K. Kuniyoshi, M. L. Wroclayski, A. Del Giglio, and F. L. A. Fonseca, “Characterization of cell-free circulating DNA in plasma in patients with prostate cancer.,” Tumour Biol., vol. 34, no. 2, pp. 983-986, April 2013.
P. Polak, R. Karlić, A. Koren, R. Thurman, R. Sandstrom, M. S. Lawrence, A. Reynolds, E. Rynes, K. Vlahoviček, J. A. Stamatoyannopoulos, and S. R. Sunyaev, “Cell-of-origin chromatin organization shapes the mutational landscape of cancer.,” Nature, vol. 518, no. 7539, pp. 360-364, February 2015.
S. El Messaoudi, F. Mouliere, S. Du Manoir, C. Bascoul-Mollevi, B. Gillet, M. Nouaille, C. Fiess, E. Crapez, F. Bibeau, C. Theillet, T. Mazard, D. Pezet, M. Mathonnet, M. Ychou, and A. R. Thierry, “Circulating DNA as a Strong Multimarker Prognostic Tool for Metastatic Colorectal Cancer Patient Management Care.,” Clin Cancer Res, vol. 22, no. 12, pp. 3067-3077, June 2016.
S. Garrigou, G. Perkins, F. Garlan, C. Normand, A. Didelot, D. Le Corre, S. Peyvandi, C. Mulot, R. Niarra, P. Aucouturier, G. Chatellier, P. Nizard, K. Perez-Toralla, E. Zonta, C. Charpy, A. Pujals, C. Barau, O. Bouché, J.-F. Emile, D. Pezet, F. Bibeau, J. B. Hutchison, D. R. Link, A. Zaanan, P. Laurent-Puig, I. Sobhani, and V. Taly, “A Study of Hypermethylated Circulating Tumor DNA as a Universal Colorectal Cancer Biomarker.,” Clin Chem, vol. 62, no. 8, pp. 1129-1139, August 2016.
S. Jahr, H. Hentze, S. Englisch, D. Hardt, F. O. Fackelmayer, R. D. Hesch, and R. Knippers, “DNA fragments in the blood plasma of cancer patients: quantitations and evidence for their origin from apoptotic and necrotic cells.,” Cancer Research, vol. 61, no. 4, pp. 1659-1665, February 2001.
Y. Li, X.-H. Fu, J.-Q. Yuan, Z.-Y. Yang, C. Mao, X.-M. Dong, J.-L. Tang, and S.-Y. Wang, “Colorectal cancer: using blood samples and tumor tissue to detect K-ras mutations.,” Expert Rev Anticancer Ther, vol. 15, no. 6, pp. 715-725, June 2015.

1. A method of screening for the methylation of a DNA region of interest, said method comprising:
contacting a DNA sample with an agent which modifies unmethylated cytosine residues wherein said sample comprises both the target strand and the opposite strand of said DNA region of interest;
(ii) contacting the DNA sample of step (i) with:
a) a first set of forward and reverse primers designed to amplify one or more fully or partially methylated forms of the modified target strand of the DNA region of interest;
b) a second set of forward and reverse primers designed to amplify one or more fully or partially methylated forms of the modified opposite strand of the DNA region of interest; and
c) if the primers of steps (a) and (b) are methylation specific then optionally one or more probes directed to each of the target and opposite strands or if the primers of steps (a) and (b) are not methylation specific then one or more methylation specific probes directed to the target and opposite strands, wherein said probes incorporate a detection means;
(iii) amplifying the DNA sample of step (ii) wherein if one or more of the probes of step (ii)(c) are used, the extension of said primers along said gene effects the detection of said hybridised probe; and
(iv) qualitatively or quantitatively analysing the detection output of step (iii).

2-64. (canceled)

65. The method of claim 1, wherein said DNA sample comprises a low copy number of said DNA region of interest.

66. The method of claim 1, wherein the methylation of one or more CpG sites within said DNA region of interest are analyzed.

67. The method of claim 1, wherein said DNA is genomic DNA.

68. The method of claim 1, wherein said DNA region is a promoter region.

69. The method of claim 1, wherein said DNA region is a mammalian gene.

70. The method of claim 1, wherein said DNA region is a large intestine neoplasm marker.

71. The method of claim 70, wherein said large intestine neoplasm marker is one or more of the genes BCAT1, IKZF1, IRF4, GRASP or CAHM or 5 kb upstream of the transcription start site.

72. The method of claim 70, wherein said large intestine neoplasm markers are selected from (i) BCAT1 and IKZF1; (ii) BCAT1, IKZF1 and IRF4; (iii) BCAT1, IKZF1 and GRASP; (iv) BCAT1, IKZF1 and CAHM; (iv) BCAT1, IKZF1, IRF4 and GRASP; (v) BCAT1, IKZF1, IRF4 and CAHM; or (vi) BCAT1, IKZF1, IRF4, GRASP and CAHM or 5 kb upstream of the transcription start sites of these genes.

73. The method of claim 1, wherein said agent is sodium bisulfite or sodium metabisulphite.

74. The method of claim 1, wherein said probes collectively hybridize to all full and partial methylation patterns at said DNA region of interest.

75. The method of claim 1, wherein said DNA region is IKZF1,
wherein said first set of primers comprise a forward primer comprising a sequence as set forth in SEQ ID NO:11 and a reverse primer comprising a sequence as set forth in SEQ ID NO:12, or substantially similar sequences and the probes directed to the amplification product of said first set of primers comprise one or more probes having a sequence as set forth in any one of SEQ ID NOs:13-21, or substantially similar sequences; and
wherein said second set of primers comprise a forward primer comprising a sequence as set forth in SEQ ID NO:77 and a reverse primer comprising a sequence as set forth in SEQ ID NO:78, or substantially similar sequences and the probes directed to the amplification product of said second set of primers comprise one or more probes having a sequence as set forth in any one of SEQ ID NOs:79-95, or substantially similar sequences.

76. The method of claim 1, wherein said DNA region is IKZF1,
wherein said first set of primers comprise a forward primer comprising a sequence as set forth in SEQ ID NO:11 and a reverse primer comprising a sequence as set forth in SEQ ID NO:12, or substantially similar sequences and the probes directed to the amplification product of said first set of primers comprise one or more probes having a sequence as set forth in any one of SEQ ID NOs:13-21, or substantially similar sequences; and
wherein said second set of primers comprise a forward primer comprising a sequence as set forth in SEQ ID NO:22 and a reverse primer comprising a sequence as set forth in SEQ ID NO:23, or substantially similar sequences and the probes directed to the amplification product of said second set of primers comprise one or more probes having a sequence as set forth in any one of SEQ ID NOs:24-32, or substantially similar sequences.

77. The method claim 1, wherein said DNA region is IKZF1,
wherein said first set of primers comprise a forward primer comprising a sequence as set forth in SEQ ID NO:11 and a reverse primer comprising a sequence as set forth in SEQ ID NO:12, or substantially similar sequences and the probes directed to the amplification product of said first set of primers comprise one or more probes having a sequence as set forth in any one of SEQ ID NOs:13-21, or substantially similar sequences; and
wherein said second set of primers comprise a forward primer comprising a sequence as set forth in SEQ ID NO:33 and a reverse primer comprising a sequence as set forth in SEQ ID NO:23, or substantially similar sequences and the probes directed to the amplification product of said second set of primers comprise one or more probes having a sequence as set forth in any one of SEQ ID NOs:34-50, or substantially similar sequences.

78. The method of claim 1, wherein said DNA region is BCAT1,
wherein said first set of primers comprise a forward primer comprising a sequence as set forth in SEQ ID NO:97 and a reverse primer comprising a sequence as set forth in SEQ ID NO:65, or substantially similar sequences and the probes directed to the amplification product of said first set of primers comprise a sequence as set forth in SEQ ID NO:66, or substantially similar sequences; and
wherein said second set of primers comprise a forward primer comprising a sequence as set forth in SEQ ID NO:96 and a reverse primer comprising a sequence as set forth in SEQ ID NO:62, or substantially similar sequences and the probes directed to the amplification product of said second set of primers comprise a sequence as set forth in SEQ ID NO:63, or substantially similar sequences.

79. The method of claim 1, wherein said DNA region is BCAT1,
wherein said first set of primers comprise a forward primer comprising a sequence as set forth in SEQ ID NO:64 and a reverse primer comprising a sequence as set forth in SEQ ID NO:65, or substantially similar sequences and the probes directed to the amplification product of said first set of primers comprise a sequence as set forth in SEQ ID NO:66, or substantially similar sequences; and
wherein said second set of primers comprise a forward primer comprising a sequence as set forth in SEQ ID NO:61 and a reverse primer comprising a sequence as set forth in SEQ ID NO:62, or substantially similar sequences and the probes directed to the amplification product of said second set of primers comprise a sequence as set forth in SEQ ID NO:63, or substantially similar sequences.

80. The method of claim 1, wherein said DNA region is IRF4,
wherein said first set of primers comprise a forward primer comprising a sequence as set forth in SEQ ID NO:108 and a reverse primer comprising a sequence as set forth in SEQ ID NO:109, or substantially similar sequences and the probes directed to the amplification product of said first set of primers comprise a sequence as set forth in SEQ ID NO:110, or substantially similar sequences; and
wherein said second set of primers comprise a forward primer comprising a sequence as set forth in SEQ ID NO:111 and a reverse primer comprising a sequence as set forth in SEQ ID NO:112, or substantially similar sequences and the probes directed to the amplification product of said second set of primers comprise one or more probes having a sequence as set forth in any one of SEQ ID NOs:113 and 115, or substantially similar sequences.

81. The method of claim 1, wherein said DNA region is IRF4,
wherein said first set of primers comprise a forward primer comprising a sequence as set forth in SEQ ID NO:108 and a reverse primer comprising a sequence as set forth in SEQ ID NO:109, or substantially similar sequences and the probes directed to the amplification product of said first set of primers comprise a sequence as set forth in SEQ ID NO:110, or substantially similar sequences; and
wherein said second set of primers comprise a forward primer comprising a sequence as set forth in SEQ ID NO:114 and a reverse primer comprising a sequence as set forth in SEQ ID NO:112, or substantially similar sequences and the probes directed to the amplification product of said second set of primers comprise one or more probes having a sequence as set forth in any one of SEQ ID NOs:113 and 115, or substantially similar sequences.

82. The method of claim 1, wherein said DNA region is IRF4,
wherein said first set of primers comprise a forward primer comprising a sequence as set forth in SEQ ID NO:108 and a reverse primer comprising a sequence as set forth in SEQ ID NO:109, or substantially similar sequences and the probes directed to the amplification product of said first set of primers comprise a sequence as set forth in SEQ ID NO:110, or substantially similar sequences; and
wherein said second set of primers comprise a forward primer comprising a sequence as set forth in SEQ ID NO:116 and a reverse primer comprising a sequence as set forth in SEQ ID NO:112, or substantially similar sequences and the probes directed to the amplification product of said second set of primers comprise one or more probes having a sequence as set forth in any one of SEQ ID NOs:113 and 115, or substantially similar sequences.

83. The method of claim 1, wherein the methylation of two or more gene regions is analyzed.

84. The method of claim 1, wherein said DNA sample is isolated from blood, plasma, serum, saliva, stool, ascites fluid or urine.

85. The method of claim 1, wherein said DNA sample is circulating cell free DNA or circulating tumor DNA.

86. The method of claim 1, wherein said amplification is PCR, reverse-transcriptase PCR, qPCR, isothermal amplification or signal amplification.

87. The method of claim 1, wherein said DNA is human DNA.


SEQUENCE LISTING (116 sequences)

SEQ ID NO: 1 - DNA, 159 bp, Homo sapiens
cgcgcgagccgggggactcggcgacggggcggggacgggacgacgcaccctctccgtgtcccgctctgcgcccttctgcgcgccccgctccctgtaccggagcagcgatccgggaggcggccgagaggtgcgcgcggggccgagccggctgcggggcag

SEQ ID NO: 2 - DNA, 159 bp, Homo sapiens
ctgccccgcagccggctcggccccgcgcgcacctctcggccgcctcccggatcgctgctccggtacagggagcggggcgcgcagaagggcgcagagcgggacacggagagggtgcgtcgtcccgtccccgccccgtcgccgagtcccccggctcgcgcg

SEQ ID NO: 3 - DNA, 159 bp, Artificial Sequence
  Bisulphite converted sequence
cgcgcgagtcgggggattcggcgacggggcggggacgggacgacgtatttttttcgtgtttcgttttgcgtttttttgcgcgtttcgttttttgtatcggagtagcgattcgggaggcggtcgagaggtgcgcgcggggtcgagtcggttgcggggtag

SEQ ID NO: 4 - DNA, 159 bp, Artificial Sequence
  Bisulphite converted sequence
ctaccccgcaaccgactcgaccccgcgcgcacctctcgaccgcctcccgaatcgctactccgatacaaaaaacgaaacgcgcaaaaaaacgcaaaacgaaacacgaaaaaaatacgtcgtcccgtccccgccccgtcgccgaatcccccgactcgcgcg

SEQ ID NO: 5 - DNA, 159 bp, Artificial Sequence
  Bisulphite converted sequence
tgtgtgagttgggggatttggtgatggggtggggatgggatgatgtattttttttgtgttttgttttgtgtttttttgtgtgttttgttttttgtattggagtagtgatttgggaggtggttgagaggtgtgtgtggggttgagttggttgtggggtag

SEQ ID NO: 6 - DNA, 159 bp, Artificial Sequence
  Bisulphite converted sequence
ctaccccacaaccaactcaaccccacacacacctctcaaccacctcccaaatcactactccaatacaaaaaacaaaacacacaaaaaaacacaaaacaaaacacaaaaaaaatacatcatcccatccccaccccatcaccaaatcccccaactcacaca

SEQ ID NO: 7 - DNA, 159 bp, Artificial Sequence
  Bisulphite converted sequence
cgcgcgaaccgaaaaactcgacgacgaaacgaaaacgaaacgacgcaccctctccgtatcccgctctacgcccttctacgcgccccgctccctataccgaaacaacgatccgaaaaacgaccgaaaaatacgcgcgaaaccgaaccgactacgaaacaa

SEQ ID NO: 8 - DNA, 159 bp, Artificial Sequence
  Bisulphite converted sequence
ttgtttcgtagtcggttcggtttcgcgcgtatttttcggtcgtttttcggatcgttgtttcggtatagggagcggggcgcgtagaagggcgtagagcgggatacggagagggtgcgtcgtttcgttttcgtttcgtcgtcgagtttttcggttcgcgcg

SEQ ID NO: 9 - DNA, 159 bp, Artificial Sequence
  Bisulphite converted sequence
cacacaaaccaaaaaactcaacaacaaaacaaaaacaaaacaacacaccctctccatatcccactctacacccttctacacaccccactccctataccaaaacaacaatccaaaaaacaaccaaaaaatacacacaaaaccaaaccaactacaaaacaa

SEQ ID NO: 10 - DNA, 159 bp, Artificial Sequence
  Bisulphite converted sequence
ttgttttgtagttggtttggttttgtgtgtattttttggttgttttttggattgttgttttggtatagggagtggggtgtgtagaagggtgtagagtgggatatggagagggtgtgttgttttgtttttgttttgttgttgagttttttggtttgtgtg

SEQ ID NO: 11 - DNA, 24 bp, Artificial Sequence
  Primer
gacgacgtatttttttcgtgtttc

SEQ ID NO: 12 - DNA, 16 bp, Artificial Sequence
  Primer
gcgcacctctcgaccg

SEQ ID NO: 13 - DNA, 27 bp, Artificial Sequence
  Probe
  Y (8)..(8): Y = T or C
  Y (16)..(16): Y = T or C
  Y (21)..(21): Y = T or C
tttgtatyggagtagygattygggagg

SEQ ID NO: 14 - DNA, 27 bp, Artificial Sequence
  Probe
tttgtatcggagtagcgattcgggagg

SEQ ID NO: 15 - DNA, 27 bp, Artificial Sequence
  Probe
tttgtatcggagtagcgatttgggagg

SEQ ID NO: 16 - DNA, 27 bp, Artificial Sequence
  Probe
tttgtatcggagtagtgattcgggagg

SEQ ID NO: 17 - DNA, 27 bp, Artificial Sequence
  Probe
tttgtattggagtagcgattcgggagg

SEQ ID NO: 18 - DNA, 27 bp, Artificial Sequence
  Probe
tttgtatcggagtagtgatttgggagg

SEQ ID NO: 19 - DNA, 27 bp, Artificial Sequence
  Probe
tttgtattggagtagcgatttgggagg

SEQ ID NO: 20 - DNA, 27 bp, Artificial Sequence
  Probe
tttgtattggagtagtgattcgggagg

SEQ ID NO: 21 - DNA, 27 bp, Artificial Sequence
  Probe
tttgtattggagtagtgatttgggagg

SEQ ID NO: 22 - DNA, 25 bp, Artificial Sequence
  Primer
ttgtttcgtagtcggttcggtttcg

SEQ ID NO: 23 - DNA, 24 bp, Artificial Sequence
  Primer
aacgacgcaccctctccgtatccc

SEQ ID NO: 24 - DNA, 27 bp, Artificial Sequence
  Probe
  Y (6)..(6): Y = T or C
  Y (11)..(11): Y = T or C
  Y (19)..(19): Y = T or C
tttttyggatygttgtttyggtatagg

SEQ ID NO: 25 - DNA, 27 bp, Artificial Sequence
  Probe
tttttcggatcgttgtttcggtatagg

SEQ ID NO: 26 - DNA, 27 bp, Artificial Sequence
  Probe
tttttcggatcgttgttttggtatagg

SEQ ID NO: 27 - DNA, 27 bp, Artificial Sequence
  Probe
tttttcggattgttgtttcggtatagg

SEQ ID NO: 28 - DNA, 27 bp, Artificial Sequence
  Probe
ttttttggatcgttgtttcggtatagg

SEQ ID NO: 29 - DNA, 27 bp, Artificial Sequence
  Probe
tttttcggattgttgttttggtatagg

SEQ ID NO: 30 - DNA, 27 bp, Artificial Sequence
  Probe
ttttttggattgttgtttcggtatagg

SEQ ID NO: 31 - DNA, 27 bp, Artificial Sequence
  Probe
ttttttggatcgttgttttggtatagg

SEQ ID NO: 32 - DNA, 27 bp, Artificial Sequence
  Probe
ttttttggattgttgttttggtatagg

SEQ ID NO: 33 - DNA, 27 bp, Artificial Sequence
  Primer
cggtcgtttttcggatcgttgtttcgg

SEQ ID NO: 34 - DNA, 21 bp, Artificial Sequence
  Probe
  Y (1)..(1): Y = T or C
  Y (3)..(3): Y = T or C
  Y (13)..(13): Y = T or C
  Y (20)..(20): Y = T or C
ygygtagaagggygtagagyg

SEQ ID NO: 35 - DNA, 21 bp, Artificial Sequence
  Probe
cgcgtagaagggcgtagagcg

SEQ ID NO: 36 - DNA, 21 bp, Artificial Sequence
  Probe
cgcgtagaagggcgtagagtg

SEQ ID NO: 37 - DNA, 21 bp, Artificial Sequence
  Probe
cgcgtagaagggtgtagagtg

SEQ ID NO: 38 - DNA, 21 bp, Artificial Sequence
  Probe
cgtgtagaagggtgtagagtg

SEQ ID NO: 39 - DNA, 21 bp, Artificial Sequence
  Probe
tgtgtagaagggtgtagagtg

SEQ ID NO: 40 - DNA, 21 bp, Artificial Sequence
  Probe
tgcgtagaagggcgtagagcg

SEQ ID NO: 41 - DNA, 21 bp, Artificial Sequence
  Probe
cgtgtagaagggcgtagagcg

SEQ ID NO: 42 - DNA, 21 bp, Artificial Sequence
  Probe
cgcgtagaagggtgtagagcg

SEQ ID NO: 43 - DNA, 21 bp, Artificial Sequence
  Probe
tgtgtagaagggcgtagagcg

SEQ ID NO: 44 - DNA, 21 bp, Artificial Sequence
  Probe
tgcgtagaagggtgtagagcg

SEQ ID NO: 45 - DNA, 21 bp, Artificial Sequence
  Probe
tgcgtagaagggcgtagagtg

SEQ ID NO: 46 - DNA, 21 bp, Artificial Sequence
  Probe
tgtgtagaagggtgtagagcg

SEQ ID NO: 47 - DNA, 21 bp, Artificial Sequence
  Probe
tgtgtagaagggcgtagagtg

SEQ ID NO: 48 - DNA, 21 bp, Artificial Sequence
  Probe
tgcgtagaagggtgtagagtg

SEQ ID NO: 49 - DNA, 21 bp, Artificial Sequence
  Probe
cgtgtagaagggcgtagagtg

SEQ ID NO: 50 - DNA, 21 bp, Artificial Sequence
  Probe
cgtgtagaagggtgtagagcg

SEQ ID NO: 51 - DNA, 107 bp, Homo sapiens
cagtgcccgaggcggcggcgagtacacgtggcgggctggattgcagaccggccctctcgcggcggagactcgcgacctagcggattgcatcagcaggaagacactaa

SEQ ID NO: 52 - DNA, 107 bp, Homo sapiens
ttagtgtcttcctgctgatgcaatccgctaggtcgcgagtctccgccgcgagagggccggtctgcaatccagcccgccacgtgtactcgccgccgcctcgggcactg

SEQ ID NO: 53 - DNA, 107 bp, Artificial Sequence
  Bisulphite converted sequence
tagtgttcgaggcggcggcgagtatacgtggcgggttggattgtagatcggttttttcgcggcggagattcgcgatttagcggattgtattagtaggaagatattaa

SEQ ID NO: 54 - DNA, 107 bp, Artificial Sequence
  Bisulphite converted sequence
ttaatatcttcctactaatacaatccgctaaatcgcgaatctccgccacgaaaaaaccgatctacaatccaacccgccacgtatactcgccgccgcctcgaacacta

SEQ ID NO: 55 - DNA, 107 bp, Artificial Sequence
  Bisulphite converted sequence
tagtgtttgaggtggtggtgagtatatgtggtgggttggattgtagattggtttttttgtggtggagatttgtgatttagtggattgtattagtaggaagatattaa

SEQ ID NO: 56 - DNA, 107 bp, Artificial Sequence
  Bisulphite converted sequence
ttaatatcttcctactaatacaatccactaaatcacaaatctccaccacaaaaaaaccaatctacaatccaacccaccacatatactcaccaccacctcaaacacta

SEQ ID NO: 57 - DNA, 107 bp, Artificial Sequence
  Bisulphite converted sequence
caatacccgaaacgacgacgaatacacgtaacgaactaaattacaaaccgaccctctcgcgacgaaaactcgcgacctaacgaattacatcaacaaaaaaacactaa

SEQ ID NO: 58 - DNA, 107 bp, Artificial Sequence
  Bisulphite converted sequence
ttagtgtttttttgttgatgtaattcgttaggtcgcgagttttcgtcgcgagagggtcggtttgtaatttagttcgttacgtgtattcgtcgtcgtttcgggtattg

SEQ ID NO: 59 - DNA, 107 bp, Artificial Sequence
  Bisulphite converted sequence
caatacccaaaacaacaacaaatacacataacaaactaaattacaaaccaaccctctcacaacaaaaactcacaacctaacaaattacatcaacaaaaaaacactaa

SEQ ID NO: 60 - DNA, 107 bp, Artificial Sequence
  Bisulphite converted sequence
ttagtgtttttttgttgatgtaatttgttaggttgtgagtttttgttgtgagagggttggtttgtaatttagtttgttatgtgtatttgttgttgttttgggtattg

SEQ ID NO: 61 - DNA, 28 bp, Artificial Sequence
  Primer
tagtgttcgaggcggcggcgagtatacg

SEQ ID NO: 62 - DNA, 29 bp, Artificial Sequence
  Primer
atcttcctactaatacaatccgctaaatc

SEQ ID NO: 63 - DNA, 20 bp, Artificial Sequence
  Probe
atcggttttttcgcggcgga

SEQ ID NO: 64 - DNA, 29 bp, Artificial Sequence
  Primer
gtttttttgttgatgtaattcgttaggtc

SEQ ID NO: 65 - DNA, 20 bp, Artificial Sequence
  Primer
caatacccgaaacgacgacg

SEQ ID NO: 66 - DNA, 21 bp, Artificial Sequence
  Probe
ttcgtcgcgagagggtcggtt

SEQ ID NO: 67 - DNA, 134 bp, Homo sapiens
ctcagccaatgggacctgctcctcccttgaaggttgcagaggccacagcctggtgggaaagatgaccaccacccagcacacagtggcagacacaggttcacagtccagaagacttgctgagcctcctccatcac

SEQ ID NO: 68 - DNA, 134 bp, Homo sapiens
gtgatggaggaggctcagcaagtcttctggactgtgaacctgtgtctgccactgtgtgctgggtggtggtcatctttcccaccaggctgtggcctctgcaaccttcaagggaggagcaggtcccattggctgag

SEQ ID NO: 69 - DNA, 19 bp, Artificial Sequence
  Primer
ggaggaggctcagcaagtc

SEQ ID NO: 70 - DNA, 18 bp, Artificial Sequence
  Primer
ctcagccaatgggacctg

SEQ ID NO: 71 - DNA, 23 bp, Artificial Sequence
  Probe
acctgtgtctgccactgtgtgct

SEQ ID NO: 72 - DNA, 89 bp, Artificial Sequence
  Bisulphite converted sequence
caaaataaaatacaaaacaaacctaatccccaaacaaaacctatattaccactaaacctccattcaactaaccaaaaaacaaaaactcc

SEQ ID NO: 73 - DNA, 89 bp, Artificial Sequence
  Bisulphite converted sequence
ggagtttttgttttttggttagttgaatggaggtttagtggtaatataggttttgtttggggattaggtttgttttgtattttattttg

SEQ ID NO: 74 - DNA, 25 bp, Artificial Sequence
  Primer
ggagtttttgttttttggttagttg

SEQ ID NO: 75 - DNA, 29 bp, Artificial Sequence
  Primer
caaaataaaatacaaaacaaacctaatcc

SEQ ID NO: 76 - DNA, 34 bp, Artificial Sequence
  Probe
atggaggtttagtggtaatataggttttgtttgg

SEQ ID NO: 77 - DNA, 20 bp, Artificial Sequence
  Primer
cgcgtagaagggcgtagagc

SEQ ID NO: 78 - DNA, 21 bp, Artificial Sequence
  Primer
gcgcgaaccgaaaaactcgac

SEQ ID NO: 79 - DNA, 24 bp, Artificial Sequence
  Probe
  Y (3)..(3): Y = T or C
  Y (6)..(6): Y = T or C
  Y (17)..(17): Y = T or C
  Y (24)..(24): Y = T or C
aaygaygcaccctctcygtatccy

SEQ ID NO: 80 - DNA, 24 bp, Artificial Sequence
  Probe
aacgacgcaccctctccgtatcct

SEQ ID NO: 81 - DNA, 24 bp, Artificial Sequence
  Probe
aatgacgcaccctctccgtatccc

SEQ ID NO: 82 - DNA, 24 bp, Artificial Sequence
  Probe
aacgatgcaccctctccgtatccc

SEQ ID NO: 83 - DNA, 24 bp, Artificial Sequence
  Probe
aacgacgcaccctctctgtatccc

SEQ ID NO: 84 - DNA, 24 bp, Artificial Sequence
  Probe
aatgatgcaccctctccgtatccc

SEQ ID NO: 85 - DNA, 24 bp, Artificial Sequence
  Probe
aatgacgcaccctctctgtatccc

SEQ ID NO: 86 - DNA, 24 bp, Artificial Sequence
  Probe
aatgacgcaccctctccgtatcct

SEQ ID NO: 87 - DNA, 24 bp, Artificial Sequence
  Probe
aacgatgcaccctctctgtatccc

SEQ ID NO: 88 - DNA, 24 bp, Artificial Sequence
  Probe
aacgatgcaccctctccgtatcct

SEQ ID NO: 89 - DNA, 24 bp, Artificial Sequence
  Probe
aacgacgcaccctctctgtatcct

SEQ ID NO: 90 - DNA, 24 bp, Artificial Sequence
  Probe
aatgatgcaccctctctgtatccc

SEQ ID NO: 91 - DNA, 24 bp, Artificial Sequence
  Probe
aatgacgcaccctctctgtatcct

SEQ ID NO: 92 - DNA, 24 bp, Artificial Sequence
  Probe
aacgatgcaccctctctgtatcct

SEQ ID NO: 93 - DNA, 24 bp, Artificial Sequence
  Probe
aatgatgcaccctctctgtatcct

SEQ ID NO: 94 - DNA, 24 bp, Artificial Sequence
  Probe
aacgacgcaccctctccgtatccc

SEQ ID NO: 95 - DNA, 24 bp, Artificial Sequence
  Probe
aatgatgcaccctctccgtatcct

SEQ ID NO: 96 - DNA, 25 bp, Artificial Sequence
  Primer
tagtgttcgaggcggcggcgagtat

SEQ ID NO: 97 - DNA, 27 bp, Artificial Sequence
  Primer
ttagtgtttttttgttgatgtaattcg

SEQ ID NO: 98 - DNA, 126 bp, Homo sapiens
cggcgcgcggagggtcgccaagggcgcgggaaccccaccccggccgcggcagcccccagccttcacgccggccctgaggctcgcccgcccggccggccccggctctcggcttgcaaagtccctctc

SEQ ID NO: 99 - DNA, 126 bp, Homo sapiens
gagagggactttgcaagccgagagccggggccggccgggcgggcgagcctcagggccggcgtgaaggctgggggctgccgcggccggggtggggttcccgcgcccttggcgaccctccgcgcgccg

SEQ ID NO: 100 - DNA, 126 bp, Artificial Sequence
  Bisulphite converted sequence
cggcgcgcggagggtcgttaagggcgcgggaattttatttcggtcgcggtagtttttagtttttacgtcggttttgaggttcgttcgttcggtcggtttcggttttcggtttgtaaagtttttttt

SEQ ID NO: 101 - DNA, 126 bp, Artificial Sequence
  Bisulphite converted sequence
aaaaaaaactttacaaaccgaaaaccgaaaccgaccgaacgaacgaacctcaaaaccgacgtaaaaactaaaaactaccgcgaccgaaataaaattcccgcgcccttaacgaccctccgcgcgccg

SEQ ID NO: 102 - DNA, 126 bp, Artificial Sequence
  Bisulphite converted sequence
tggtgtgtggagggttgttaagggtgtgggaattttattttggttgtggtagtttttagtttttatgttggttttgaggtttgtttgtttggttggttttggtttttggtttgtaaagtttttttt

SEQ ID NO: 103 - DNA, 126 bp, Artificial Sequence
  Bisulphite converted sequence
aaaaaaaactttacaaaccaaaaaccaaaaccaaccaaacaaacaaacctcaaaaccaacataaaaactaaaaactaccacaaccaaaataaaattcccacacccttaacaaccctccacacacca

SEQ ID NO: 104 - DNA, 126 bp, Artificial Sequence
  Bisulphite converted sequence
cgacgcgcgaaaaatcgccaaaaacgcgaaaaccccaccccgaccgcgacaacccccaaccttcacgccgaccctaaaactcgcccgcccgaccgaccccgactctcgacttacaaaatccctctc

SEQ ID NO: 105 - DNA, 126 bp, Artificial Sequence
  Bisulphite sequence
gagagggattttgtaagtcgagagtcggggtcggtcgggcgggcgagttttagggtcggcgtgaaggttgggggttgtcgcggtcggggtggggttttcgcgtttttggcgatttttcgcgcgtcg

SEQ ID NO: 106 - DNA, 126 bp, Artificial Sequence
  Bisulphite converted sequence
caacacacaaaaaatcaccaaaaacacaaaaaccccaccccaaccacaacaacccccaaccttcacaccaaccctaaaactcacccacccaaccaaccccaactctcaacttacaaaatccctctc

SEQ ID NO: 107 - DNA, 126 bp, Artificial Sequence
  Bisulphite converted sequence
gagagggattttgtaagttgagagttggggttggttgggtgggtgagttttagggttggtgtgaaggttgggggttgttgtggttggggtggggtttttgtgtttttggtgattttttgtgtgttg

SEQ ID NO: 108 - DNA, 19 bp, Artificial Sequence
  Primer
ggcgcgggaattttatttc

SEQ ID NO: 109 - DNA, 18 bp, Artificial Sequence
  Primer
aaaccgaccgaacgaacg

SEQ ID NO: 110 - DNA, 32 bp, Artificial Sequence
  Probe
aaaaccgacgtaaaaactaaaaactaccgcga

SEQ ID NO: 111 - DNA, 23 bp, Artificial Sequence
  Primer
taagtcgagagtcggggtcggtc

SEQ ID NO: 112 - DNA, 16 bp, Artificial Sequence
  Primer
cgacgcgcgaaaaatc

SEQ ID NO: 113 - DNA, 28 bp, Artificial Sequence
  Probe
ccaaccttcacgccgaccctaaaactcg

SEQ ID NO: 114 - DNA, 17 bp, Artificial Sequence
  Primier
gagagtcggggtcggtc

SEQ ID NO: 115 - DNA, 24 bp, Artificial Sequence
  Probe
ccttcacgccgaccctaaaactcg

SEQ ID NO: 116 - DNA, 26 bp, Artificial Sequence
  Primer
gagagggattttgtaagtcgagagtc